(19) United States
(12) Patent Application Publication
(10) Pub. No.: US 2022/0223801 A1
(43) Pub. Date: Jul. 14, 2022

(54) MATERIALS FOR ORGANIC ELECTROLUMINESCENT DEVICES

(71) Applicant: Merck Patent GmbH, Darmstadt (DE)

(72) Inventors: Rouven LINGE, Darmstadt (DE); Lara-Isabel RODRIGUEZ, Darmstadt (DE); Aaron LACKNER, Mannheim (DE); Sebastian MEYER, Frankfurt am Main (DE); Amel MEKIC, Darmstadt (DE)

(21) Appl. No.: 17/610,816

(22) PCT Filed: Oct. 28, 2019
(86) PCT No.: PCT/EP2019/079331

§ 371 Date: Nov. 12, 2021

(30) Foreign Application Priority Data
Oct. 31, 2018 (EP) 18203716.8

(51) Int. Cl. H01L 51/00; C07D 493/04; C09K 11/06; C07D 495/04

(52) U.S. Cl. CPC H01L 51/0073; C07D 493/04; C09K 11/06; H01L 51/5012; H01L 51/0052; C07D 495/04; H01L 51/0074; H01L 51/0058

(57) ABSTRACT

The present invention relates to compounds of the formula (1) which are suitable for use in electronic devices, in particular organic electroluminescent devices, to a process for manufacturing the compounds of formula (1), to intermediate compounds for manufacturing the compounds of formula (1) and to electronic devices comprising the compounds of formula (1).

The present invention relates to a compound of the formula (1), to the use of the compound in an electronic device, and to an electronic device comprising a compound of the formula (1). The present invention furthermore relates to a process for the preparation of a compound of the formula (1), to intermediates used in the preparation of a compound of formula (1) and to a formulation comprising one or more compounds of the formula (1).

The development of functional compounds for use in electronic devices is currently the subject of intensive research. The aim is, in particular, the development of compounds with which improved properties of electronic devices in one or more relevant points can be achieved, such as, for example, power efficiency and lifetime of the device as well as colour coordinates of the emitted light.

In accordance with the present invention, the term electronic device is taken to mean, inter alia, organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-lasers) and organic electroluminescent devices (OLEDs).

Of particular interest is the provision of compounds for use in the last-mentioned electronic devices called OLEDs. The general structure and the functional principle of OLEDs are known to the person skilled in the art and are described, for example, in U.S. Pat. No. 4,539,507.

Further improvements are still necessary with respect to the performance data of OLEDs, in particular with a view to broad commercial use, for example in display devices or as light sources. Of particular importance in this connection are the lifetime, the efficiency and the operating voltage of the OLEDs and as well as the colour values achieved. In particular, in case of blue-emitting OLEDs, there is potential for improvement with respect to the efficiency, lifetime and operating voltage of the devices.

An important starting point for achieving the said improvements is the choice of the emitter compound, but also of the matrix material for the emitter (also called host compound) employed in the electronic device.

Matrix materials for fluorescent emitters that are known from the prior art are a multiplicity of compounds. Compounds comprising at least one anthracene group and at least one dibenzofuran or dibenzothiophene group are known from the prior art (for example WO 2010/151006, US 2014/0027741 and US 2010/0032658).

However, there is still a need for further fluorescent emitters and further matrix materials for fluorescent emitters, which may be employed in OLEDs and lead to OLEDs having very good properties in terms of lifetime, color emission and efficiency. More particularly, there is a need for matrix materials for fluorescent emitters combining very high efficiencies, very good life time and very good thermal stability.

Furthermore, it is known that an OLED may comprise different layers, which may be applied either by vapour deposition in a vacuum chamber or by processing from a solution. The processes based on vapour deposition lead to very good results, but they might be complex and expensive. Therefore, there is also a need for OLED materials that can be easily and reliably processed from solution. In this case, the materials should have good solubility properties in the solution that comprises them.

There is furthermore still a need for processes, which lead to stable OLED materials, which are easily purified and easily processed. There is a need for processes, which are economically and qualitatively interesting by providing OLED materials in acceptable purity and with a high yield.

The present invention is thus based on the technical object of providing compounds which are suitable for use in electronic devices, such as OLEDs, more particularly as matrix materials for fluorescent emitters or as fluorescent emitters, which are suitable for vacuum processing or for solution processing.

The present invention is also based on the technical object of providing processes and intermediate compounds for the manufacturing of OLED materials.

In investigations on novel compounds for use in electronic devices, it has now been found, that compounds of formula (1) as defined below are eminently suitable for use in electronic devices. In particular, they achieve one or more, preferably all, of the above-mentioned technical objects.

The invention thus relates to compounds of formula (1),

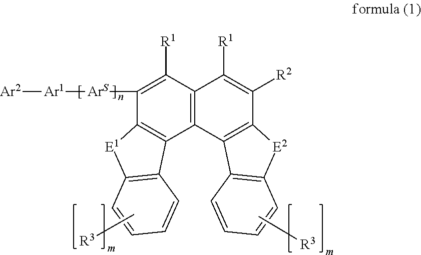

where the following applies to the symbols and indices used:
Ar¹ is on each occurrence, identically or differently, a condensed aryl or heteroaryl group having 10 to 18 aromatic ring atoms, which may be substituted by one or more radicals R;
Ar² is on each occurrence, identically or differently, an aromatic or heteroaromatic ring system having 5 to 60 aromatic ring atoms, which may in each case be substituted by one or more radicals R;
Ar^{S} is on each occurrence, identically or differently, an aromatic or heteroaromatic ring system having 5 to 30 aromatic ring atoms, which may in each case be substituted by one or more radicals R;
E¹, E² are on each occurrence, identically or differently, selected from —BR⁰—, —C(R⁰)₂—, —Si(R⁰)₂—, —C(═O)—, —O—, —S—, —S(═O)—, —SO₂—, —N(R⁰)—, and —P(R⁰)—;
R¹ stands on each occurrence, identically or differently, for H, D, F, Cl, Br, I, CHO, CN, N(Ar)₂, C(═O)Ar, P(═O)(Ar)₂, S(═O)Ar, S(═O)₂Ar, NO₂, Si(R)₃, B(OR)₂, OSO₂R, a straight-chain alkyl, alkoxy or thioalkyl group having 1 to 40 C atoms or branched or cyclic alkyl, alkoxy or thioalkyl group having 3 to 40 C atoms, each of which may be substituted by one or more radicals R, where in each case one or more non-adjacent CH₂ groups may be replaced by RC═CR, C≡C, Si(R)₂, Ge(R)₂, Sn(R)₂, C═O, C═S, C═Se, P(═O)(R), SO, SO₂, O, S or CONR and where one or more H atoms may be replaced by D, F, Cl, Br, I, CN or NO₂, an aromatic or heteroaromatic ring system having 5 to 60 aromatic ring atoms, which may in each case be substituted by one or more radicals R, or an aryloxy group having 5 to 60 aromatic ring atoms, which may be substituted by one or more radicals R, where two substituents R¹ may form a mono- or polycyclic, aliphatic ring system or aromatic ring system, which may be substituted by one or more radicals R;
R², R³ stand on each occurrence, identically or differently, for
   H, D, F, Cl, Br, I, CHO, CN, N(Ar)₂, C(═O)Ar, P(═O)(Ar)₂, S(═O)Ar, S(═O)₂Ar, NO₂, Si(R)₃, B(OR)₂, OSO₂R;
   a straight-chain alkyl, alkoxy or thioalkyl group having 1 to 40 C atoms or branched or cyclic alkyl, alkoxy or thioalkyl group having 3 to 40 C atoms, each of which may be substituted by one or more radicals R, where in each case one or more non-adjacent CH₂ groups may be replaced by RC═CR, C≡C, Si(R)₂, Ge(R)₂, Sn(R)₂, C═O, C═S, C═Se, P(═O)(R), SO, SO₂, O, S or CONR and where one or more H atoms may be replaced by D, F, Cl, Br, I, CN or NO₂;
   an aromatic or heteroaromatic ring system having 5 to 60 aromatic ring atoms, which may in each case be substituted by one or more radicals R;
   an aryloxy group having 5 to 60 aromatic ring atoms, which may be substituted by one or more radicals R; or
   for a group of the following formula:

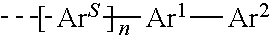

where the dashed bond indicates the bond to the structure of formula (1);
and where one substituent R² and one adjacent substituent R¹ and/or two substituents R³ may form a mono- or polycyclic, aliphatic ring system or aromatic ring system, which may be substituted by one or more radicals R; or for
m stands on each occurrence, identically or differently, for an integer selected from 0, 1, 2, 3 or 4;
n stands on each occurrence, identically or differently, for an integer selected from 0, 1, 2, 3 or 4;
R stands on each occurrence, identically or differently, for H, D, F, Cl, Br, I, CHO, CN, N(Ar)₂, C(═O)Ar, P(═O)(Ar)₂, S(═O)Ar, S(═O)₂Ar, NO₂, Si(R′)₃, B(OR′)₂, OSO₂R′, a straight-chain alkyl, alkoxy or thioalkyl group having 1 to 40 C atoms or branched or cyclic alkyl, alkoxy or thioalkyl group having 3 to 40 C atoms, each of which may be substituted by one or more radicals R′, where in each case one or more non-adjacent CH₂ groups may be replaced by R′C═CR′, C≡C, Si(R′)₂, Ge(R′)₂, Sn(R′)₂, C═O, C═S, C═Se, P(═O)(R′), SO, SO₂, O, S or CONR′ and where one or more H atoms may be replaced by D, F, Cl, Br, I, CN or NO₂, an aromatic or heteroaromatic ring system having 5 to 60 aromatic ring atoms, which may in each case be substituted by one or more radicals R′, or an aryloxy group having 5 to 60 aromatic ring atoms, which may be substituted by one or more radicals R′, where two substituents R may form a mono- or polycyclic, aliphatic ring system or aromatic ring system, which may be substituted by one or more radicals R′;
Ar is an aromatic or heteroaromatic ring system having 5 to 24 aromatic ring atoms, which may in each case also be substituted by one or more radicals R′;
R′ stands on each occurrence, identically or differently, for H, D, F, Cl, Br, I, CN, a straight-chain alkyl, alkoxy or thioalkyl group having 1 to 20 C atoms or branched or cyclic alkyl, alkoxy or thioalkyl group having 3 to 20 C atoms, where in each case one or more non-adjacent CH₂ groups may be replaced by SO, SO₂, O, S and where one or more H atoms may be replaced by D, F, Cl, Br or I, or an aromatic or heteroaromatic ring system having 5 to 24 C atoms.

Adjacent substituents in the sense of the present invention are substituents which are bonded to atoms which are linked directly to one another or which are bonded to the same atom.

Furthermore, the following definitions of chemical groups apply for the purposes of the present application:

An aryl group in the sense of this invention contains 6 to 60 aromatic ring atoms, preferably 6 to 40 aromatic ring atoms, more preferably 6 to 20 aromatic ring atoms; a heteroaryl group in the sense of this invention contains 5 to 60 aromatic ring atoms, preferably 5 to 40 aromatic ring atoms, more preferably 5 to 20 aromatic ring atoms, at least one of which is a heteroatom. The heteroatoms are preferably selected from N, O and S. This represents the basic definition. If other preferences are indicated in the description of the present invention, for example with respect to the number of aromatic ring atoms or the heteroatoms present, these apply.

An aryl group or heteroaryl group here is taken to mean either a simple aromatic ring, i.e. benzene, or a simple heteroaromatic ring, for example pyridine, pyrimidine or thiophene, or a condensed (annellated) aromatic or heteroaromatic polycycle, for example naphthalene, phenanthrene, quinoline or carbazole. A condensed (annellated) aromatic or heteroaromatic polycycle in the sense of the present application consists of two or more simple aromatic or heteroaromatic rings condensed with one another.

An aryl or heteroaryl group, which may in each case be substituted by the above-mentioned radicals and which may be linked to the aromatic or heteroaromatic ring system via any desired positions, is taken to mean, in particular, groups derived from benzene, naphthalene, anthracene, phenanthrene, pyrene, dihydropyrene, chrysene, perylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aryloxy group in accordance with the definition of the present invention is taken to mean an aryl group, as defined above, which is bonded via an oxygen atom. An analogous definition applies to heteroaryloxy groups.

An aromatic ring system in the sense of this invention contains 6 to 60 C atoms in the ring system, preferably 6 to 40 C atoms, more preferably 6 to 20 C atoms. A heteroaromatic ring system in the sense of this invention contains 5 to 60 aromatic ring atoms, preferably 5 to 40 aromatic ring atoms, more preferably 5 to 20 aromatic ring atoms, at least one of which is a heteroatom. The heteroatoms are preferably selected from N, O and/or S. An aromatic or heteroaromatic ring system in the sense of this invention is intended to be taken to mean a system which does not necessarily contain only aryl or heteroaryl groups, but instead in which, in addition, a plurality of aryl or heteroaryl groups may be connected by a non-aromatic unit (preferably less than 10% of the atoms other than H), such as, for example, an sp³-hybridised C, Si, N or O atom, an sp²-hybridised C or N atom or an sp-hybridised C atom. Thus, for example, systems such as 9,9′-spirobifluorene, 9,9′-diarylfluorene, triarylamine, diaryl ether, stilbene, etc., are also intended to be taken to be aromatic ring systems in the sense of this invention, as are systems in which two or more aryl groups are connected, for example, by a linear or cyclic alkyl, alkenyl or alkynyl group or by a silyl group. Furthermore, systems in which two or more aryl or heteroaryl groups are linked to one another via single bonds are also taken to be aromatic or heteroaromatic ring systems in the sense of this invention, such as, for example, systems such as biphenyl, terphenyl or diphenyltriazine.

An aromatic or heteroaromatic ring system having 5-60 aromatic ring atoms, which may in each case also be substituted by radicals as defined above and which may be linked to the aromatic or heteroaromatic group via any desired positions, is taken to mean, in particular, groups derived from benzene, naphthalene, anthracene, benzanthracene, phenanthrene, benzophenanthrene, pyrene, chrysene, perylene, fluoranthene, naphthacene, pentacene, benzopyrene, biphenyl, biphenylene, terphenyl, terphenyl-ene, quaterphenyl, fluorene, spirobifluorene, dihydrophenanthrene, dihydropyrene, tetrahydropyrene, cis- or trans-indenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, indolocarbazole, indenocarbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or combinations of these groups.

For the purposes of the present invention, a straight-chain alkyl group having 1 to 40 C atoms or a branched or cyclic alkyl group having 3 to 40 C atoms or an alkenyl or alkynyl group having 2 to 40 C atoms, in which, in addition, individual H atoms or CH₂ groups may be substituted by the groups mentioned above under the definition of the radicals, is preferably taken to mean the radicals methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl. An alkoxy or thioalkyl group having 1 to 40 C atoms is preferably taken to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoro-methylthio, pentafluoroethylthio, 2,2,2-trifluoroethylthio, ethenylthio, propenyl-thio, butenylthio, pentenylthio, cyclopentenylthio, hexenylthio, cyclohexenylthio, heptenylthio, cycloheptenylthio, octenylthio, cyclooctenyl-thio, ethynylthio, propynylthio, butynylthio, pentynylthio, hexynylthio, heptynyl-thio or octynylthio.

The formulation that two or more radicals may form a ring with one another is, for the purposes of the present application, intended to be taken to mean, inter alia, that the two radicals are linked to one another by a chemical bond. This is illustrated by the following schemes:

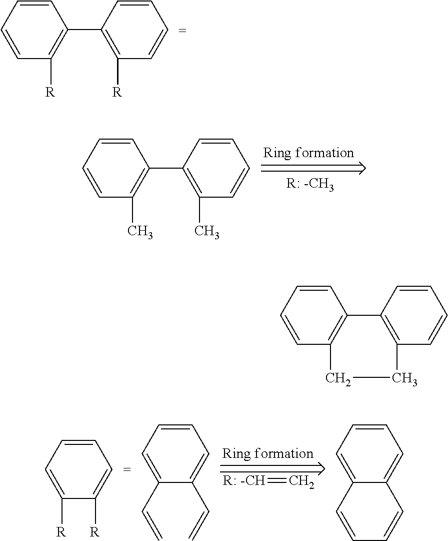

Furthermore, however, the above-mentioned formulation is also intended to be taken to mean that, in the case where one of the two radicals represents hydrogen, the second radical is bonded at the position to which the hydrogen atom was bonded, with formation of a ring. This is illustrated by the following scheme:

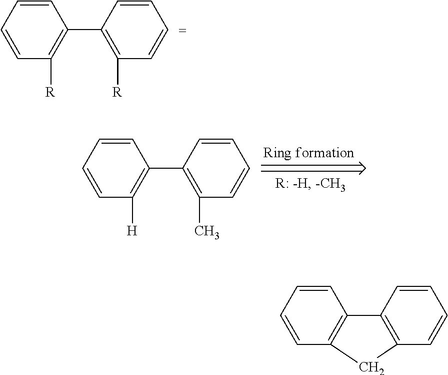

In accordance with a preferred embodiment, the compounds of formula (1) are selected from compounds of formulae (2) and (3),

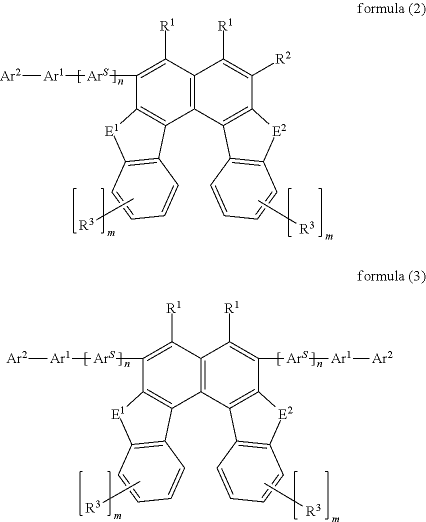

where
R², R³ stand on each occurrence, identically or differently, for H, D, F, Cl, Br, I, CHO, CN, N(Ar)₂, C(═O)Ar, P(═O)(Ar)₂, S(═O)Ar, S(═O)₂Ar, NO₂, Si(R)₃, B(OR)₂, OSO₂R, a straight-chain alkyl, alkoxy or thioalkyl group having 1 to 40 C atoms or branched or cyclic alkyl, alkoxy or thioalkyl group having 3 to 40 C atoms, each of which may be substituted by one or more radicals R, where in each case one or more non-adjacent CH₂ groups may be replaced by RC═CR, C≡C, Si(R)₂, Ge(R)₂, Sn(R)₂, C═O, C═S, C═Se, P(═O)(R), SO, SO₂, O, S or CONR and where one or more H atoms may be replaced by D, F, Cl, Br, I, CN or NO₂, an aromatic or heteroaromatic ring system having 5 to 60 aromatic ring atoms, which may in each case be substituted by one or more radicals R, or an aryloxy group having 5 to 60 aromatic ring atoms, which may be substituted by one or more radicals R, where one substituent R² and one adjacent substituent R¹ and/or two substituents R³ may form a mono- or polycyclic, aliphatic ring system or aromatic ring system, which may be substituted by one or more radicals R; and

where the symbols R¹, E¹, E², Ar¹, Ar² and Ar^{S} and the indices m and n have the same meaning as above.

Preferably, the group Ar¹ is on each occurrence, identically or differently, a condensed aryl group having 10 to 18 aromatic ring atoms. More preferably, the group Ar¹ is selected from the group consisting of anthracene, naphthalene, phenanthrene, tetracene, chrysene, benzanthracene, benzo-phenanthracene, pyrene, perylene, triphenylene, benzopyrene, fluoranthene, each of which may be substituted by one or more radicals R at any free positions. Very preferably, the group Ar¹ is an anthracene group.

Examples of suitable groups Ar¹ are the groups of formulae (Ar1-1) to (Ar1-11) as represented in the table below:

| | |
|---|---|
| 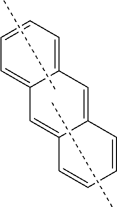 | (Ar1-1) |
| 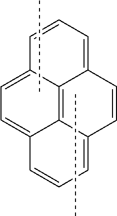 | (Ar1-2) |
| 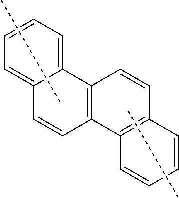 | (Ar1-3) |
| 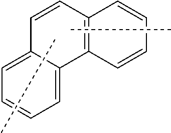 | (Ar4-4) |
| 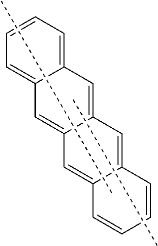 | (Ar1-5) |
| 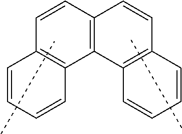 [SMILES: c1ccc2c(c1)ccc1ccc3ccccc3c12] | (Ar1-6) |
| 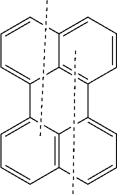 | (Ar1-7) |
| 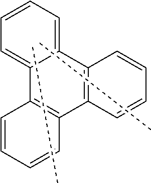 | (Ar4-8) |
| 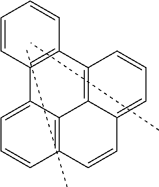 [SMILES: c1ccc2c(c1)c1cccc3ccc4cccc2c4c31] | (Ar1-9) |
| 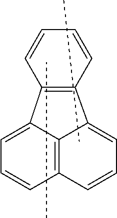 [SMILES: c1ccc2c(c1)-c1cccc3cccc-2c13] | (Ar1-10) |
| 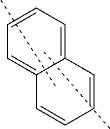 | (Ar1-11) |

where

the dashed bonds indicate the bonding to the adjacent group in formula (1); and where the groups of formulae (Ar1-1) to (Ar1-11) may be substituted at each free position by a group R, which has the same meaning as defined above.

Among the groups of formulae (Ar1-1) to (Ar1-11), the groups of formula (Ar1-1) are preferred.

Examples of very suitable groups Ar¹ are the groups of formulae (Ar1-1-1) to (Ar1-12-1) as represented in the table below:

| | |
|---|---|
| 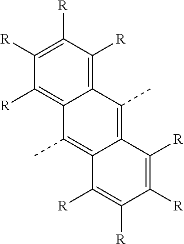 | (Ar1-1-1) |
| 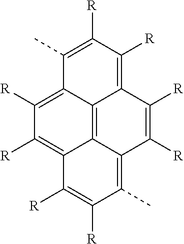 | (Ar1-2-1) |
| 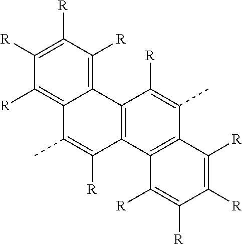 | (Ar1-3-1) |
| 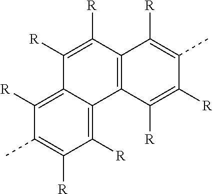 | (Ar1-4-1) |
| 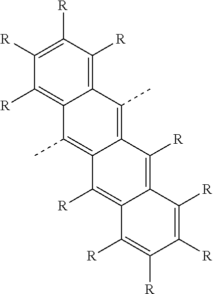 | (Ar1-5-1) |
| 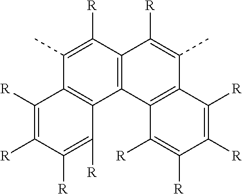 | (Ar1-6-1) |
| 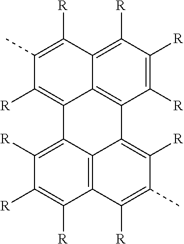 | (Ar1-7-1) |
| 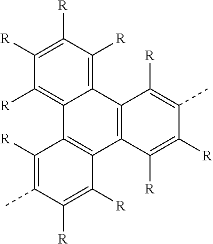 | (Ar1-8-1) |
| 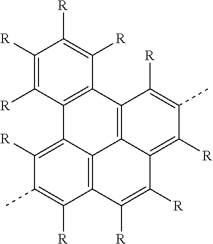 | (Ar1-9-1) |
| 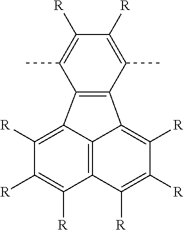 | (Ar1-10-1) |
| 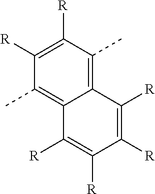 | (Ar1-11-1) |
| 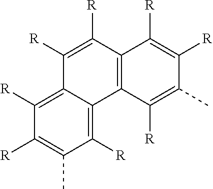 | (Ar1-12-1) |

Among the groups of formulae (Ar1-1-1) to (Ar1-12-1), the groups of formulae (Ar1-1-1) are preferred.

In accordance with a very preferred embodiment, the compounds of formula (1) are selected from the compounds of formula (2-1) or (3-1),

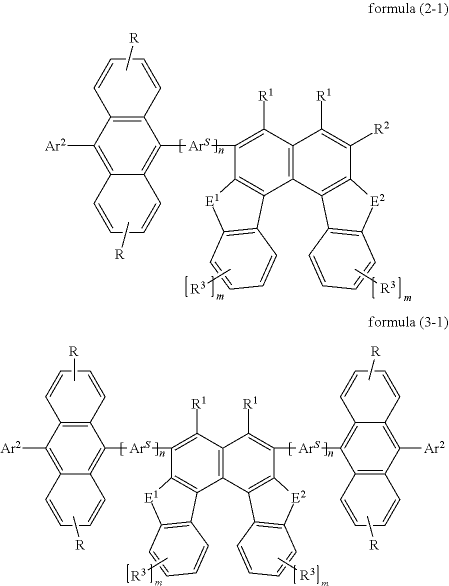

where

R², R³ stand on each occurrence, identically or differently, for H, D, F, Cl, Br, I, CHO, CN, N(Ar)₂, C(═O)Ar, P(═O)(Ar)₂, S(═O)Ar, S(═O)₂Ar, NO₂, Si(R)₃, B(OR)₂, OSO₂R, a straight-chain alkyl, alkoxy or thioalkyl group having 1 to 40 C atoms or branched or cyclic alkyl, alkoxy or thioalkyl group having 3 to 40 C atoms, each of which may be substituted by one or more radicals R, where in each case one or more non-adjacent CH₂ groups may be replaced by RC═CR, C≡C, Si(R)₂, Ge(R)₂, Sn(R)₂, C═O, C═S, C═Se, P(═O)(R), SO, SO₂, O, S or CONR and where one or more H atoms may be replaced by D, F, Cl, Br, I, CN or NO₂, an aromatic or heteroaromatic ring system having 5 to 60 aromatic ring atoms, which may in each case be substituted by one or more radicals R, or an aryloxy group having 5 to 60 aromatic ring atoms, which may be substituted by one or more radicals R, where one substituent R² and one adjacent substituent R¹ and/or two substituents R³ may form a mono- or polycyclic, aliphatic ring system or aromatic ring system, which may be substituted by one or more radicals R; and

where the symbols R, R¹, E¹, E², Ar² and Ar^{S} and the indices m and n have the same meaning as above.

Preferably, the groups E¹ and E² are on each occurrence, identically or differently, selected from —C(R⁰)₂—, —O—, —S— and —N(R⁰)—, more preferably selected from —C(R⁰)₂—, —O— and —S— and particularly preferably deleted from —O— and —S—.

In accordance with a preferred embodiment, E¹ and E² both stand for —O—.

In accordance with another preferred embodiment, E¹ and E² both stand for —S—.

In accordance with a preferred embodiment, n stands on each occurrence, identically or differently, for 0, 1 or 2.

In accordance with a particularly preferred embodiment, the compound of formula (1) are selected from the compounds of formulae (2-1-1) to (3-1-6),

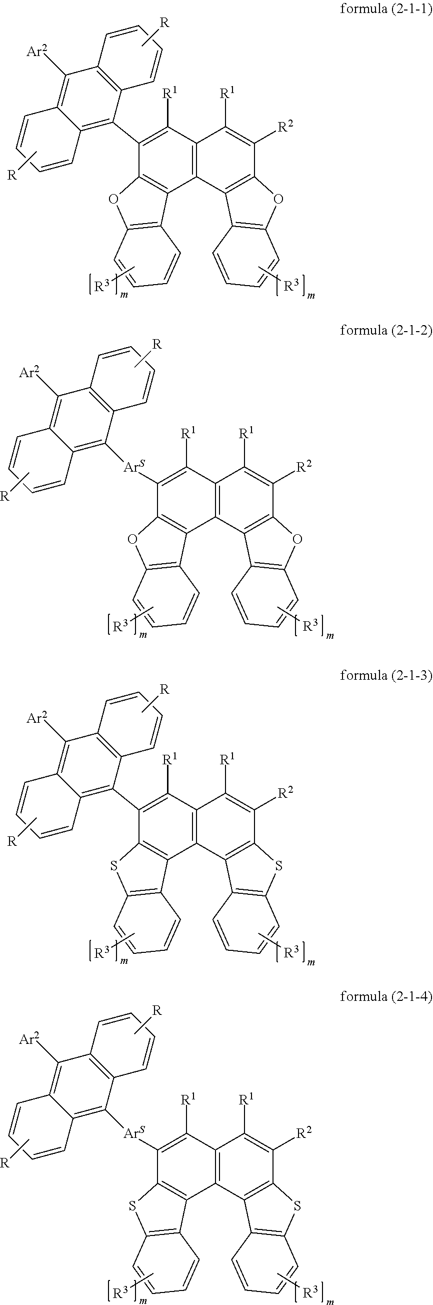

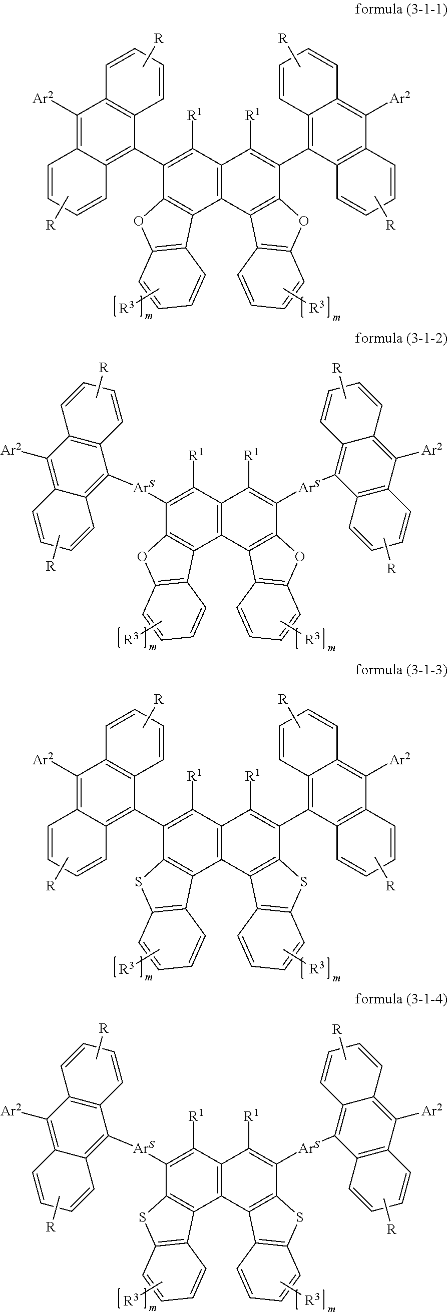

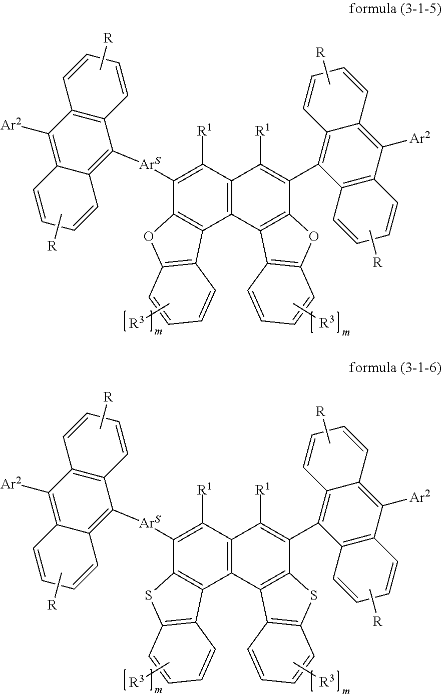

where
R², R³ stand on each occurrence, identically or differently, for H, D, F, Cl, Br, I, CHO, CN, N(Ar)₂, C(═O)Ar, P(═O)(Ar)₂, S(═O)Ar, S(═O)₂Ar, NO₂, Si(R)₃, B(OR)₂, OSO₂R, a straight-chain alkyl, alkoxy or thioalkyl group having 1 to 40 C atoms or branched or cyclic alkyl, alkoxy or thioalkyl group having 3 to 40 C atoms, each of which may be substituted by one or more radicals R, where in each case one or more non-adjacent CH₂ groups may be replaced by RC═CR, C≡C, Si(R)₂, Ge(R)₂, Sn(R)₂, C═O, C═S, C═Se, P(═O)(R), SO, SO₂, O, S or CONR and where one or more H atoms may be replaced by D, F, Cl, Br, I, CN or NO₂, an aromatic or heteroaromatic ring system having 5 to 60 aromatic ring atoms, which may in each case be substituted by one or more radicals R, or an aryloxy group having 5 to 60 aromatic ring atoms, which may be substituted by one or more radicals R, where one substituent R² and one adjacent substituent R¹ and/or two substituents R³ may form a mono- or polycyclic, aliphatic ring system or aromatic ring system, which may be substituted by one or more radicals R; and
   where the symbols R, R¹, Ar² and Ar^{S} and the index m have the same meaning as above.

In accordance with a particularly preferred embodiment, the compounds of formula (1) selected from the compounds of formulae (2-1-5) to (3-1-12),

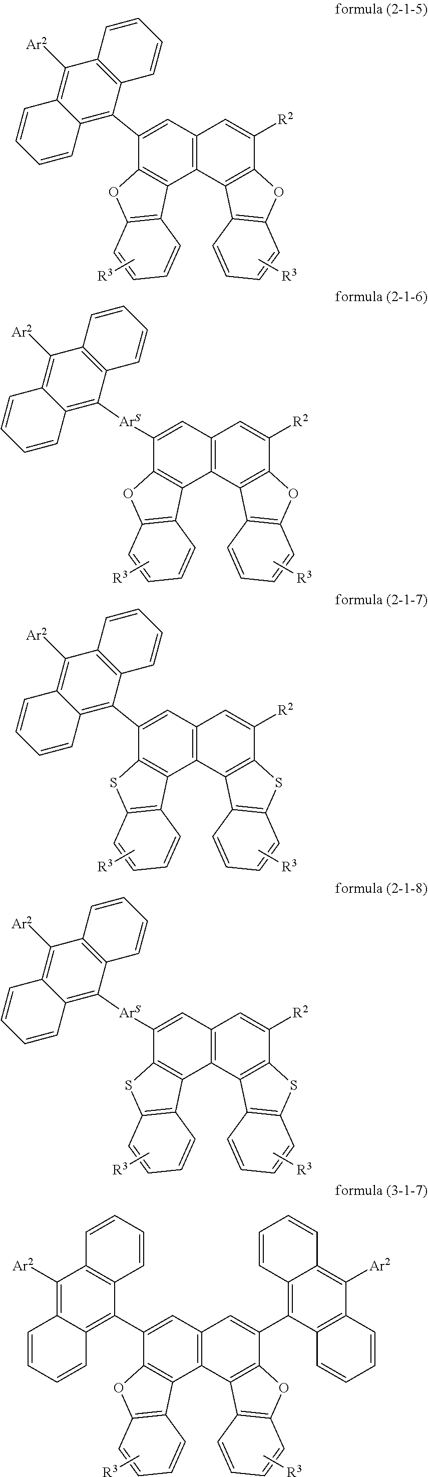

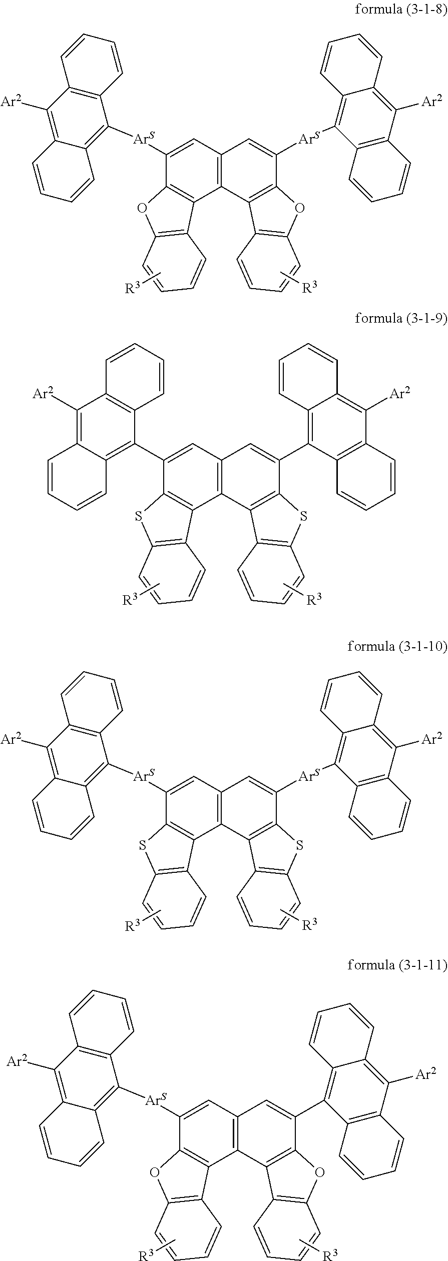

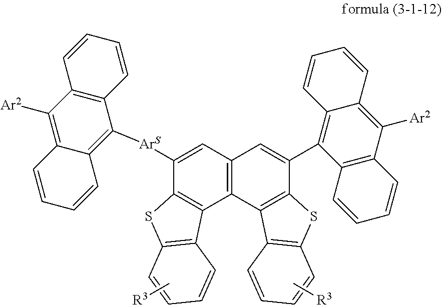

where
R², R³ stand on each occurrence, identically or differently, for H, D, F, Cl, Br, I, CHO, CN, N(Ar)₂, C(═O)Ar, P(═O)(Ar)₂, S(═O)Ar, S(═O)₂Ar, NO₂, Si(R)₃, B(OR)₂, OSO₂R, a straight-chain alkyl, alkoxy or thioalkyl group having 1 to 40 C atoms or branched or cyclic alkyl, alkoxy or thioalkyl group having 3 to 40 C atoms, each of which may be substituted by one or more radicals R, where in each case one or more non-adjacent CH₂ groups may be replaced by RC═CR, C≡C, Si(R)₂, Ge(R)₂, Sn(R)₂, C═O, C═S, C═Se, P(═O)(R), SO, SO₂, O, S or CONR and where one or more H atoms may be replaced by D, F, Cl, Br, I, CN or NO₂, an aromatic or heteroaromatic ring system having 5 to 60 aromatic ring atoms, which may in each case be substituted by one or more radicals R, or an aryloxy group having 5 to 60 aromatic ring atoms, which may be substituted by one or more radicals R, where one substituent R² and one adjacent substituent R¹ and/or two substituents R³ may form a mono- or polycyclic, aliphatic ring system or aromatic ring system, which may be substituted by one or more radicals R; and

where the symbols R, R¹, Ar² and Ar^{S} have the same meaning as in claim 1.

Preferably, the group Ar^{S} stands on each occurrence, identically or differently, for phenyl, biphenyl, fluorene, spirobifluorene, naphthalene, phenanthrene, anthracene, dibenzofuran, dibenzothiophene, carbazole, pyridine, pyrimidine, pyrazine, pyridazine, triazine, benzopyridine, benzopyridazine, benzopyrimidine and quinazoline, each of which may be substituted by one or more radicals R.

Examples of suitable groups Ar^{S} are the groups of formulae (ArS-1) to (ArS-26) as represented in the table below:

| | |
|---|---|
| 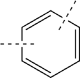 | (ArS-1) |
| 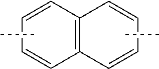 | (ArS-2) |
| 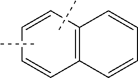 | (ArS-3) |
| 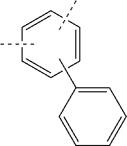 | (ArS-4) |
| 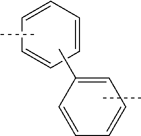 | (ArS-5) |
| 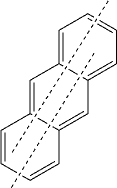 | (ArS-6) |
| 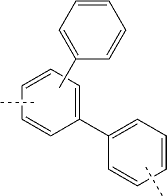 | (ArS-7) |
| 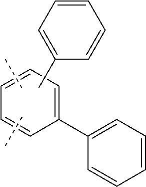 | (ArS-8) |
| 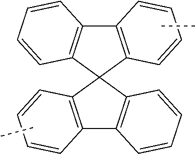 | (ArS-9) |
| 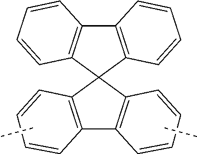 | (ArS-10) |
| 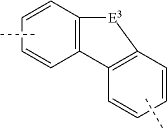 | (ArS-11) |
| 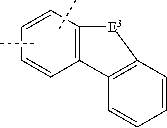 | (ArS-12) |
| 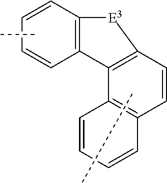 | (ArS-13) |
| 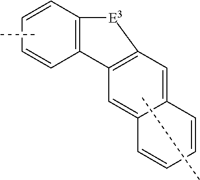 | (ArS-14) |
| 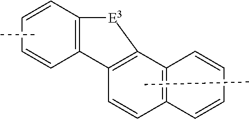 | (ArS-15) |
| 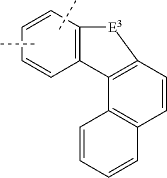 | (ArS-16) |
| 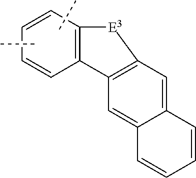 | (ArS-17) |
| 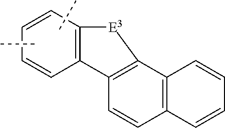 | (ArS-18) |
| 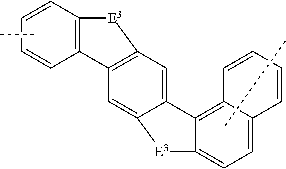 | (ArS-19 |
| 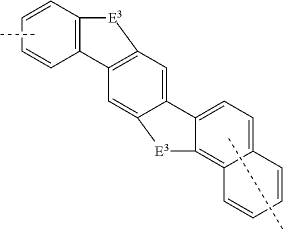 | (ArS-20) |
| 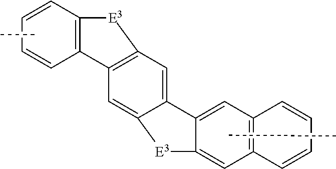 | (ArS-21) |
| 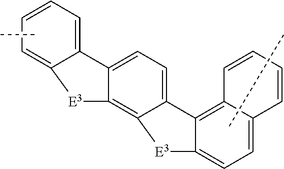 | (ArS-22) |
| 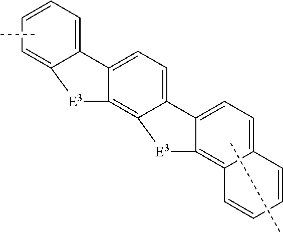 | (ArS-23) |
| 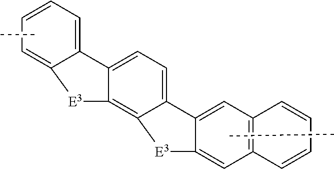 | (ArS-24) |
| 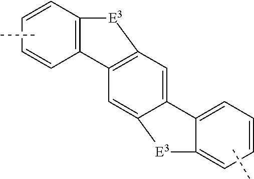 | (ArS-25) |
| 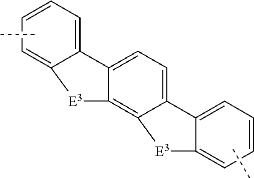 | (ArS-26) |

where the dashed bonds indicate the bonding to the adjacent groups in formula (1);

where the groups of formulae (ArS-1) to (ArS-26) may be substituted at each free position by a group R, which has the same meaning as defined above; and

where the group E³ is on each occurrence, identically or differently, selected from —BR⁰—, —C(R⁰)₂—, —Si(R⁰)₂—, —C(═O)—, —O—, —S—, —S(═O)—, —SO₂—, —N(R⁰)—, and —P(R⁰)—, where R⁰ is as defined above. Preferably, the group E³ is identically or differently, selected from —C(R⁰)₂—, —O—, —S— and —N(R⁰)—, where R⁰ is as defined above.

Among the groups of formulae (ArS-1) to (ArS-26), the groups of formulae (ArS-1), (ArS-2), (ArS-3), (ArS-11) and (ArS-12) are preferred. The groups of formula (ArS-1), (ArS-2), (ArS-3) are very preferred.

Preferably, the group Ar² is selected from aromatic or heteroaromatic ring systems having 5 to 30, preferably 5 to 25 aromatic ring atoms, which may in each case be substituted by one or more radicals R. More preferably, the group Ar² is selected from the group consisting of phenyl, biphenyl, terphenyl, quaterphenyl, fluorene, spirobifluorene, naphthalene, phenanthrene, anthracene, triphenylene, fluoranthene, tetracene, chrysene, benzanthracene, benzophenanthracene, pyrene, perylene, indole, benzofuran, benzothiophene, dibenzofuran, dibenzothiophene, carbazole, indenocarbazole, indolocarbazole, pyridine, pyrimidine, pyrazine, pyridazine, triazine, quinolone, benzopyridine, benzopyridazine, benzopyrimidine, benzimidazole and quinazoline, each of which may be substituted by one or more radicals R. More preferably, the group Ar² is selected from the group consisting of phenyl, biphenyl, terphenyl, quaterphenyl, fluorene, naphthalene, phenanthrene, triphenylene, fluoranthene, tetracene, chrysene, benzanthracene, benzophenanthracene, pyrene or perylene, each of which may be substituted by one or more radicals R at any free positions.

Examples of suitable groups Ar2 are the groups of formulae (Ar2-1) to (Ar2-27) as depicted in the table below:

| | |
|---|---|
| 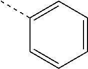 | (Ar2-1) |
| 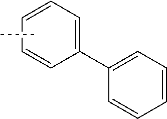 | (Ar2-2) |
| 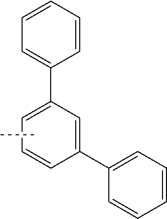 | (Ar2-3) |
| 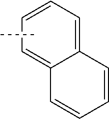 | (Ar2-4) |
| 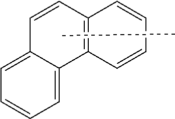 | (Ar2-5) |
| 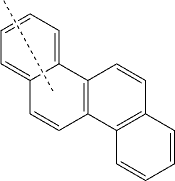 | (Ar2-6) |
| 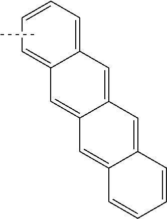 | (Ar2-7) |
| 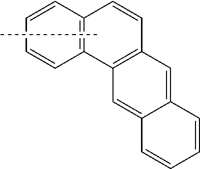 | (Ar2-8) |
| 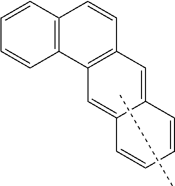 | (Ar2-9) |
| 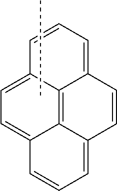 | (Ar2-10) |
| 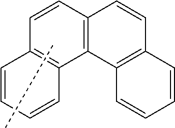 | (Ar2-11) |
| 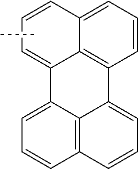 | (Ar2-12) |
| 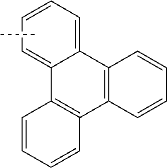 | (Ar2-13) |
| 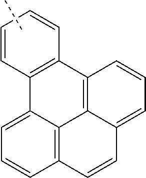 | (Ar2-14) |
| 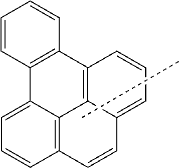 | (Ar2-15) |
| 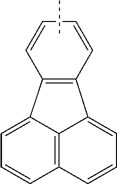 | (Ar2-16) |
| 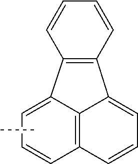 | (Ar2-17) |
| 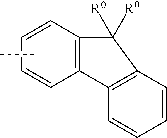 | (Ar2-18) |
| 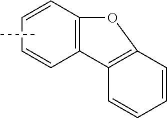 | (Ar2-19) |
| 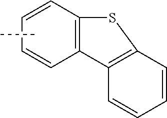 | (Ar2-20) |
| 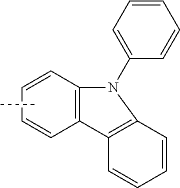 | (Ar2-21) |
| 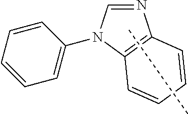 | (Ar2-22) |
| 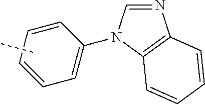 | (Ar2-23) |
| 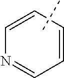 | (Ar2-24) |
| 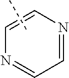 | (Ar2-25) |
| 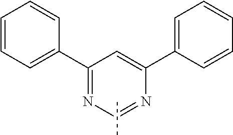 | (Ar2-26) |
| 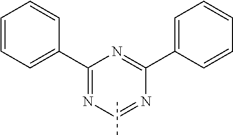 | (Ar2-27) |

where the dashed bond indicates the bonding to Ar¹ and where the group R⁰ has the same meaning as above; and where the groups of formulae (Ar2-1) to (Ar2-27) may be substituted at each free position by a group R, which has the same meaning as above.

Among the groups of formulae (Ar2-1) to (Ar2-27), the groups of formulae (Ar2-1), (Ar2-2), (Ar2-3), (Ar2-4), (Ar2-5), (Ar2-8), (Ar2-18), (Ar2-19) are preferred. The groups of formula (Ar2-1), (Ar2-2), (Ar2-3), (Ar2-4), (Ar2-5) are very preferred.

In accordance with a preferred embodiment, R⁰ stands on each occurrence, identically or differently, for H, D, F, a straight-chain alkyl group having 1 to 20, preferably 1 to 10 C atoms or branched or a cyclic alkyl group having 3 to 20, preferably 3 to 10 C atoms, each of which may be substituted by one or more radicals R, where in each case one or more non-adjacent CH₂ groups may be replaced by O or S and where one or more H atoms may be replaced by D or F, or an aromatic or heteroaromatic ring systems having 5 to 40, preferably 5 to 30, more preferably 6 to 18 aromatic ring atoms, which may in each case be substituted by one or more radicals R, where two adjacent radicals R⁰, may form an aliphatic or aromatic ring system together, which may be substituted by one or more radicals R.

Preferably, R¹, R² and R³ stand on each occurrence, identically or differently, for H, D, F, CN, N(Ar)₂, a straight-chain alkyl, alkoxy or thioalkyl groups having 1 to 40, preferably 1 to 20, more preferably 1 to 10 C atoms or branched or a cyclic alkyl, alkoxy or thioalkyl groups having 3 to 40, preferably 3 to 20, more preferably 3 to 10 C atoms, each of which may be substituted by one or more radicals R, where in each case one or more non-adjacent CH₂ groups may be replaced by RC═CR, C≡C, O or S and where one or more H atoms may be replaced by D or F, or an aromatic or heteroaromatic ring systems having 5 to 60, preferably 5 to 40, more preferably 5 to 30, particularly preferably 6 to 18 aromatic ring atoms, which may in each case be substituted by one or more radicals R, where two radicals R¹ and/or one radical R¹ and one radical R² and/or two radicals R³ may form an aliphatic or aromatic ring system together, which may be substituted by one or more radicals R. More preferably, R¹, R² and R³ stand on each occurrence, identically or differently, for H, D, F, a straight-chain alkyl group having 1 to 10 C atoms or branched or a cyclic alkyl group having 3 to 10 C atoms, each of which may be substituted by one or more radicals R, where in each case one or more H atoms may be replaced by D or F, or an aromatic or heteroaromatic ring systems having 5 to 30, preferably 6 to 18 aromatic ring atoms, which may in each case be substituted by one or more radicals R, where two radicals R¹ and/or one radical R¹ and one radical R² and/or two radicals R³ may form an aliphatic or aromatic ring system together, which may be substituted by one or more radicals R. Particularly preferably, R¹, R² and R³ stand for H.

Preferably, R stands on each occurrence, identically or differently, for H, D, F, CN, N(Ar)₂, a straight-chain alkyl, alkoxy or thioalkyl groups having 1 to 40, preferably 1 to 20, more preferably 1 to 10 C atoms or branched or a cyclic alkyl, alkoxy or thioalkyl groups having 3 to 40, preferably 3 to 20, more preferably 3 to 10 C atoms, each of which may be substituted by one or more radicals R′, where in each case one or more non-adjacent CH₂ groups may be replaced by R′C═CR′, C≡C, O or S and where one or more H atoms may be replaced by D or F, or an aromatic or heteroaromatic ring systems having 5 to 60, preferably 5 to 40, more preferably 5 to 30, particularly preferably 6 to 18 aromatic ring atoms, which may in each case be substituted by one or more radicals R′.

Preferably, R′ stands on each occurrence, identically or differently, for H, D, F, Cl, Br, I, CN, a straight-chain alkyl group having 1 to 10 C atoms or branched or cyclic alkyl group having 3 to 10 C atoms, where in each case one or more H atoms may be replaced by D or F, or an aromatic or heteroaromatic ring system having 5 to 18 C atoms.

The following compounds are examples of compounds of formula (1):

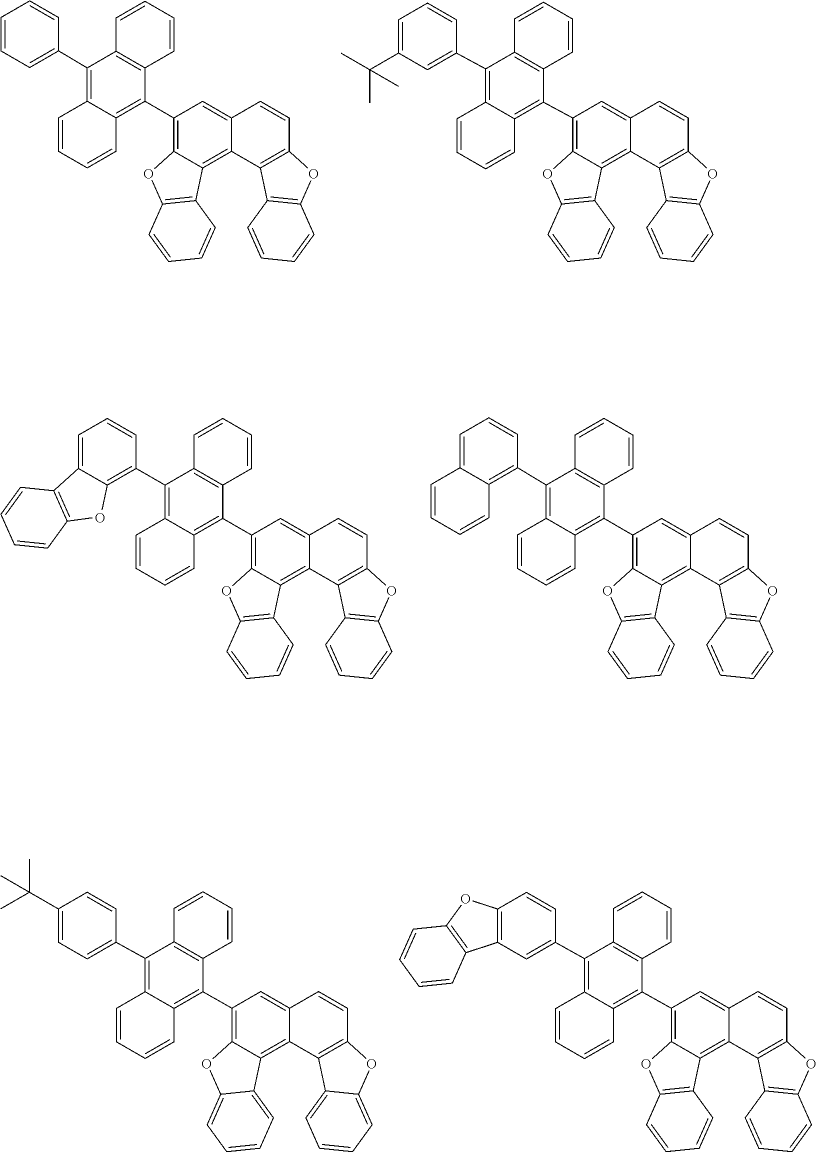 [SMILES: c1ccc(-c2c3ccccc3c(-c3cc4ccc5oc6ccccc6c5c4c4c3oc3ccccc34)c3ccccc23)cc1 ; CC(C)(C)c1cccc(-c2c3ccccc3c(-c3cc4ccc5oc6ccccc6c5c4c4c3oc3ccccc34)c3ccccc23)c1 ; c1ccc2c(c1)oc1c(-c3c4ccccc4c(-c4cc5ccc6oc7ccccc7c6c5c5c4oc4ccccc45)c4ccccc34)cccc12 ; c1ccc2c(-c3c4ccccc4c(-c4cc5ccc6oc7ccccc7c6c5c5c4oc4ccccc45)c4ccccc34)cccc2c1 ; CC(C)(C)c1ccc(-c2c3ccccc3c(-c3cc4ccc5oc6ccccc6c5c4c4c3oc3ccccc34)c3ccccc23)cc1 ; c1ccc2c(c1)oc1ccc(-c3c4ccccc4c(-c4cc5ccc6oc7ccccc7c6c5c5c4oc4ccccc45)c4ccccc34)cc12]

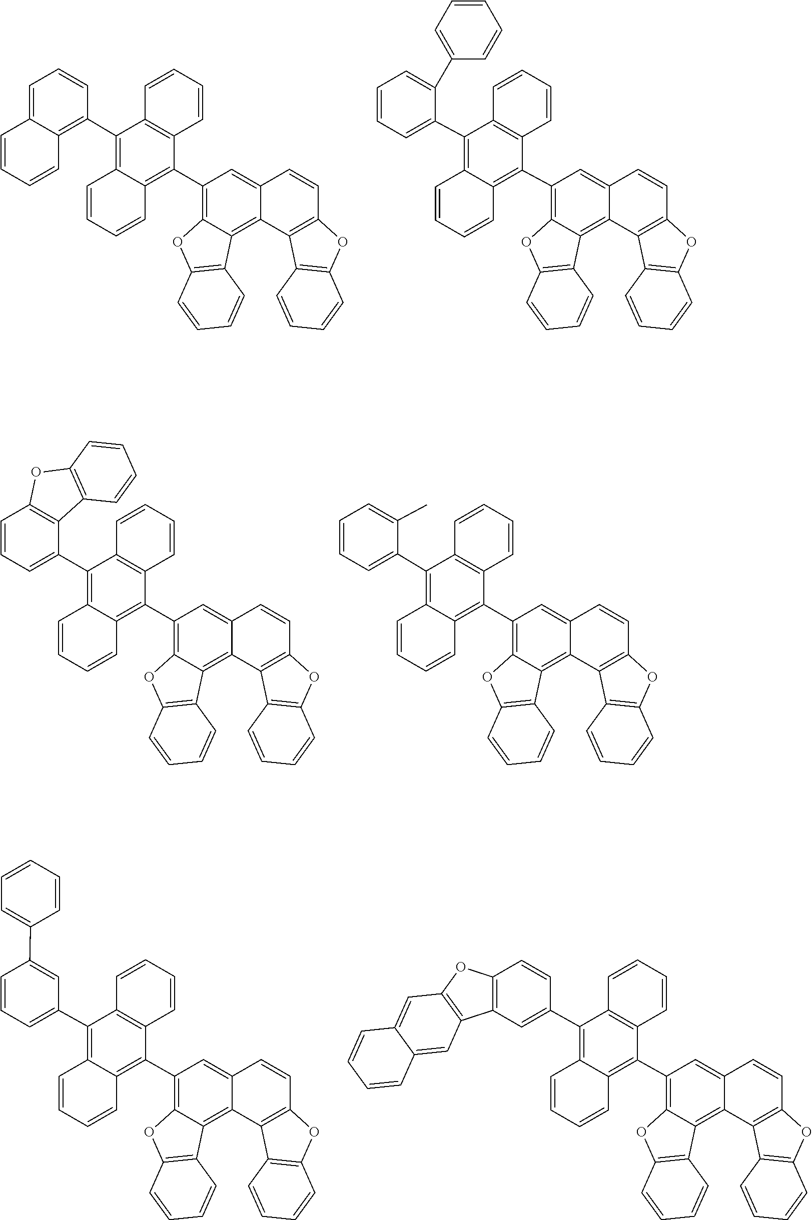 [SMILES: c1ccc2c(-c3c4ccccc4c(-c4cc5ccc6oc7ccccc7c6c5c5c4oc4ccccc45)c4ccccc34)cccc2c1 ; c1ccc(-c2ccccc2-c2c3ccccc3c(-c3cc4ccc5oc6ccccc6c5c4c4c3oc3ccccc34)c3ccccc23)cc1 ; c1ccc2c(c1)oc1cccc(-c3c4ccccc4c(-c4cc5ccc6oc7ccccc7c6c5c5c4oc4ccccc45)c4ccccc34)c12 ; Cc1ccccc1-c1c2ccccc2c(-c2cc3ccc4oc5ccccc5c4c3c3c2oc2ccccc23)c2ccccc12 ; c1ccc(-c2cccc(-c3c4ccccc4c(-c4cc5ccc6oc7ccccc7c6c5c5c4oc4ccccc45)c4ccccc34)c2)cc1 ; c1ccc2cc3c(cc2c1)oc1ccc(-c2c4ccccc4c(-c4cc5ccc6oc7ccccc7c6c5c5c4oc4ccccc45)c4ccccc24)cc13]

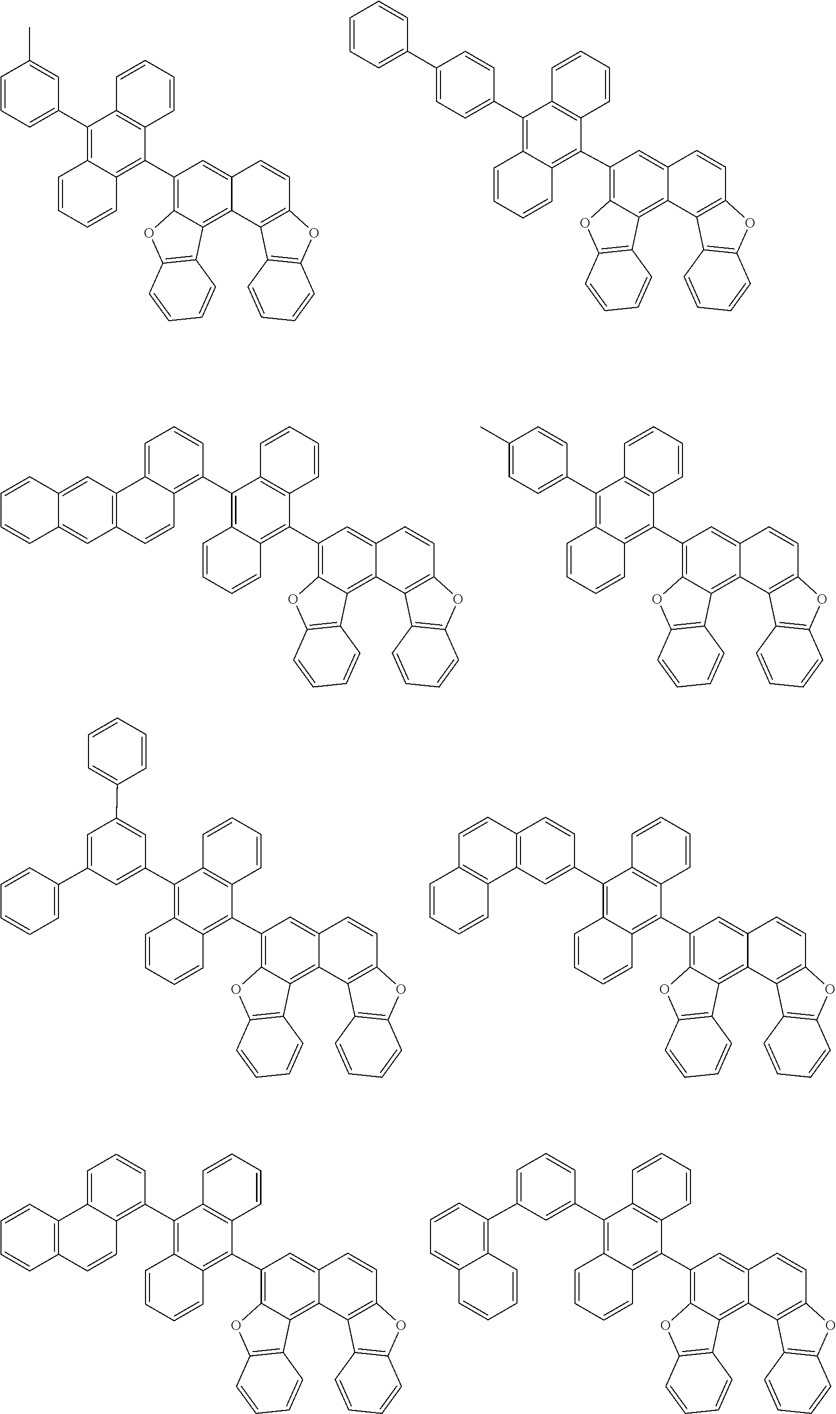

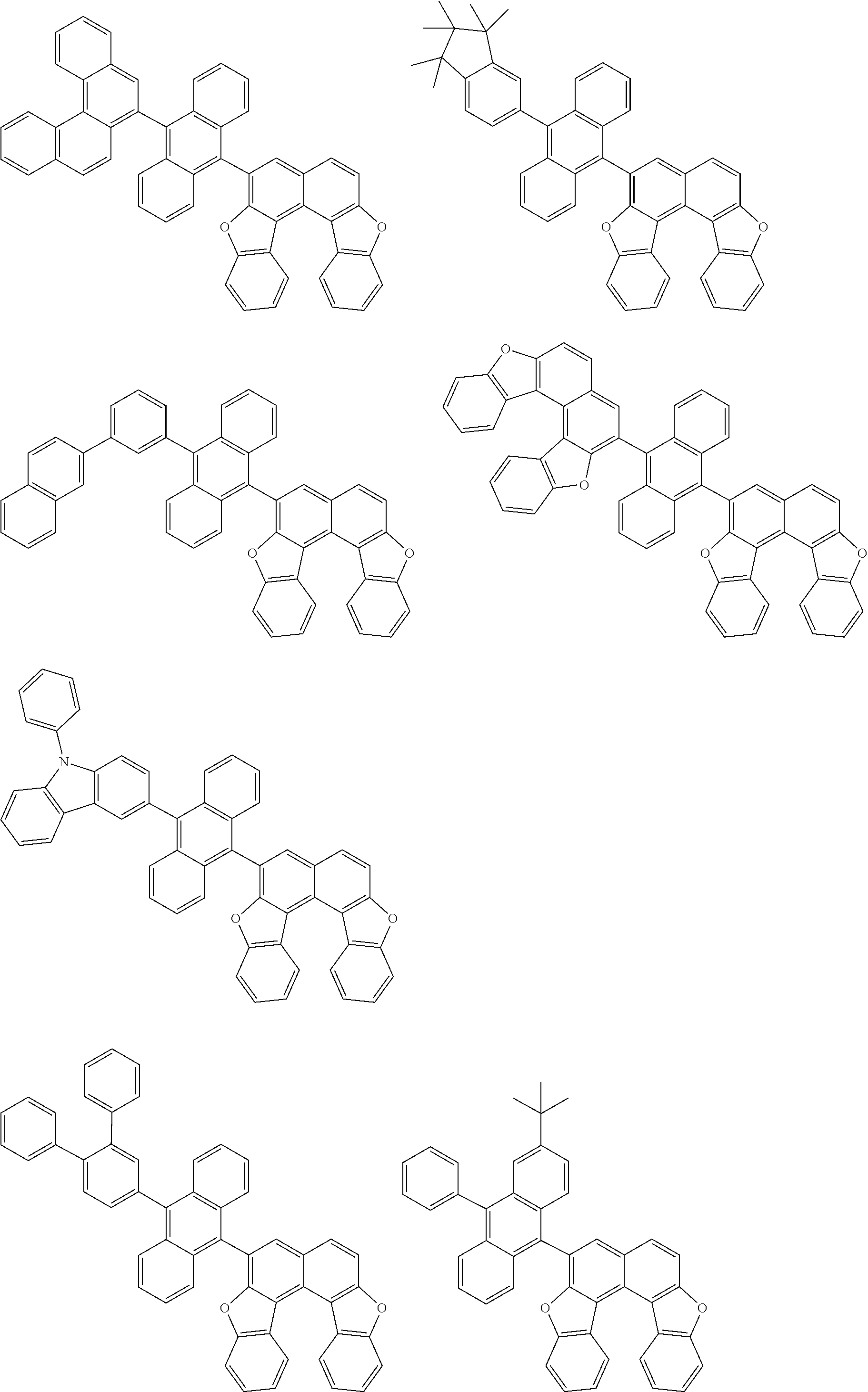

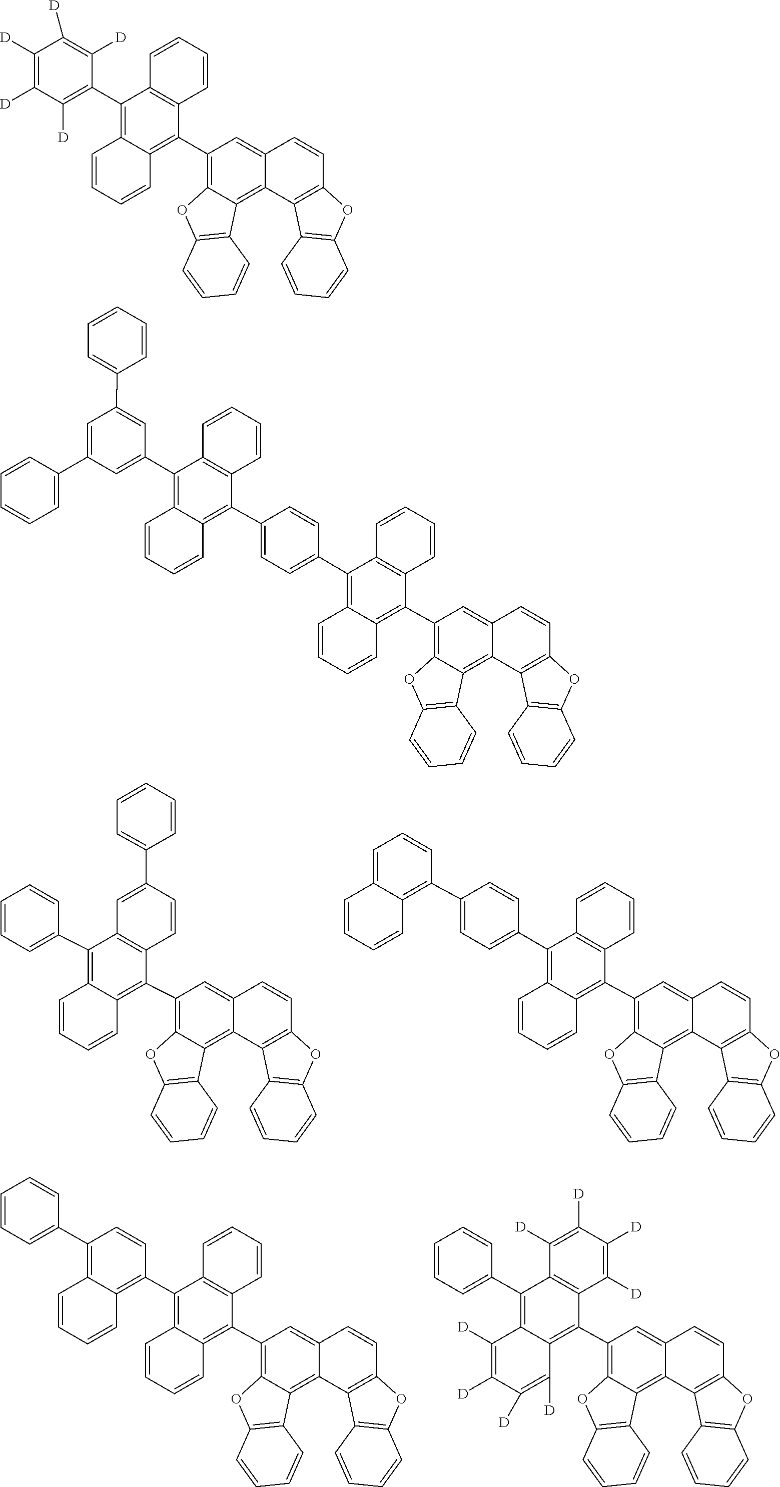

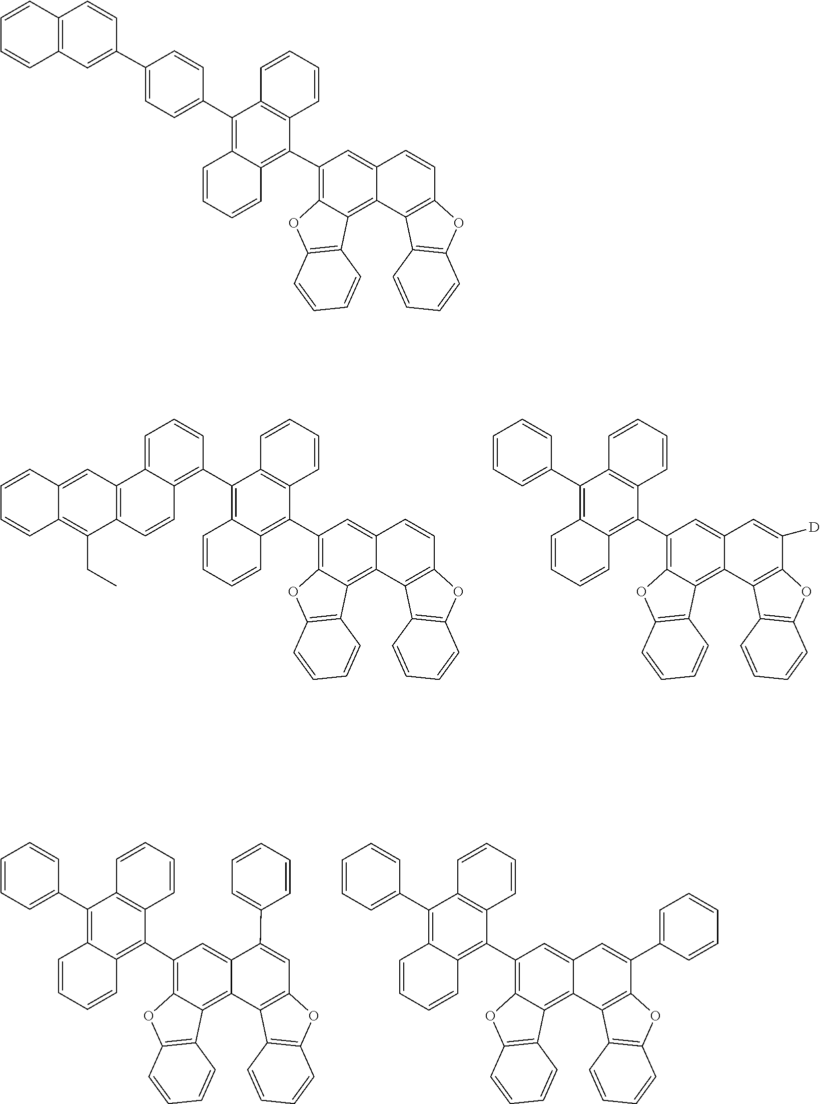 [SMILES: c1ccc2cc(-c3ccc(-c4c5ccccc5c(-c5cc6ccc7oc8ccccc8c7c6c6c5oc5ccccc56)c5ccccc45)cc3)ccc2c1 ; CCc1c2ccccc2cc2c1ccc1c(-c3c4ccccc4c(-c4cc5ccc6oc7ccccc7c6c5c5c4oc4ccccc45)c4ccccc34)cccc12 ; [2H]c1cc2cc(-c3c4ccccc4c(-c4ccccc4)c4ccccc34)c3oc4ccccc4c3c2c2c1oc1ccccc12 ; c1ccc(-c2c3ccccc3c(-c3cc4c(-c5ccccc5)cc5oc6ccccc6c5c4c4c3oc3ccccc34)c3ccccc23)cc1 ; c1ccc(-c2c3ccccc3c(-c3cc4cc(-c5ccccc5)c5oc6ccccc6c5c4c4c3oc3ccccc34)c3ccccc23)cc1]

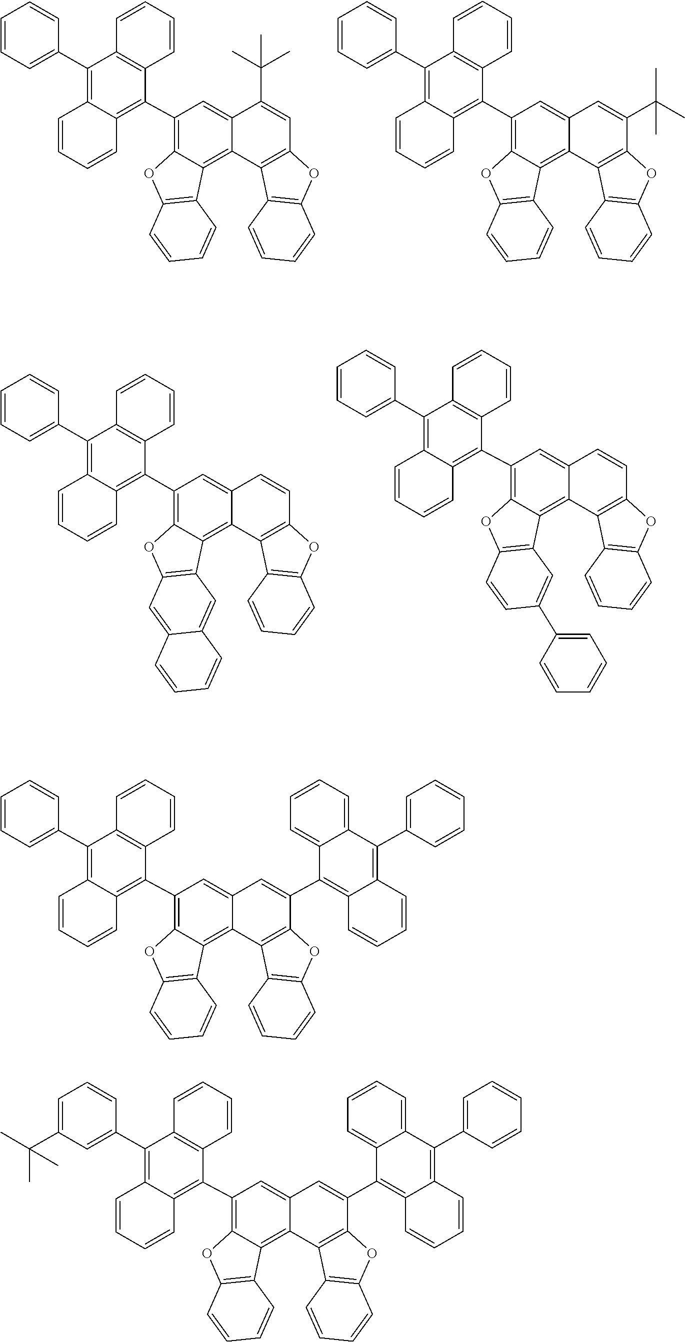

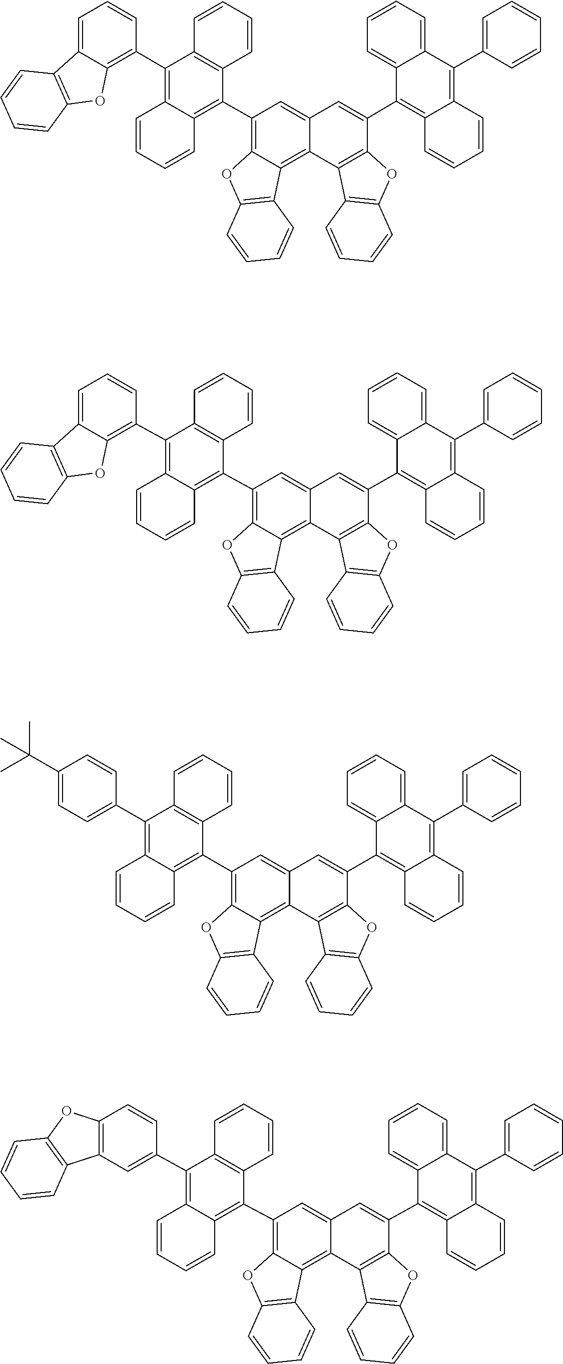 [SMILES: c1ccc(-c2c3ccccc3c(-c3cc4cc(-c5c6ccccc6c(-c6cccc7c6oc6ccccc67)c6ccccc56)c5oc6ccccc6c5c4c4c3oc3ccccc34)c3ccccc23)cc1 ; c1ccc(-c2c3ccccc3c(-c3cc4cc(-c5c6ccccc6c(-c6cccc7c6oc6ccccc67)c6ccccc56)c5oc6ccccc6c5c4c4c3oc3ccccc34)c3ccccc23)cc1 ; CC(C)(C)c1ccc(-c2c3ccccc3c(-c3cc4cc(-c5c6ccccc6c(-c6ccccc6)c6ccccc56)c5oc6ccccc6c5c4c4c3oc3ccccc34)c3ccccc23)cc1 ; c1ccc(-c2c3ccccc3c(-c3cc4cc(-c5c6ccccc6c(-c6ccc7oc8ccccc8c7c6)c6ccccc56)c5oc6ccccc6c5c4c4c3oc3ccccc34)c3ccccc23)cc1]

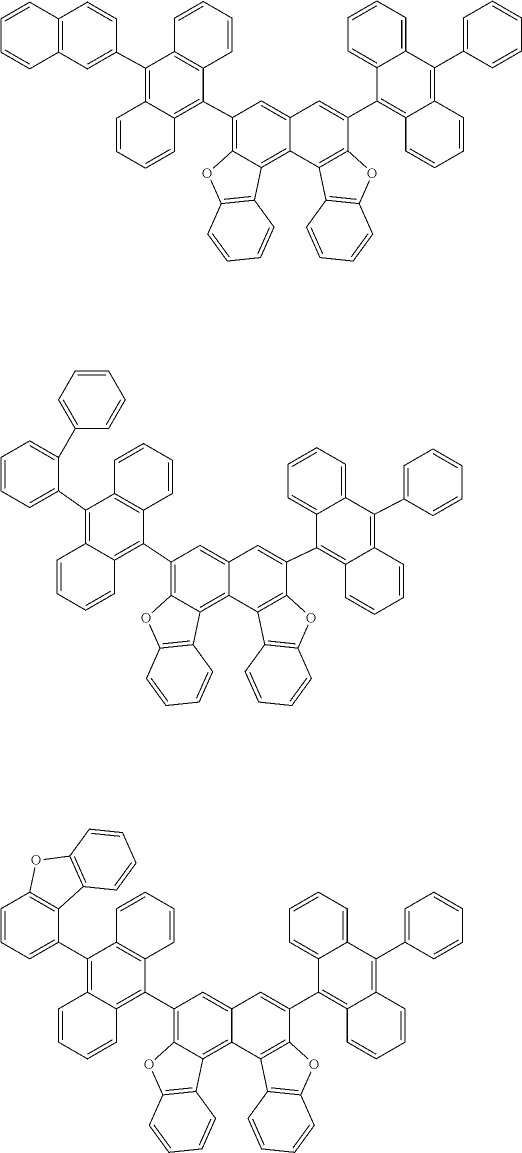 [SMILES: c1ccc(-c2c3ccccc3c(-c3cc4cc(-c5c6ccccc6c(-c6ccc7ccccc7c6)c6ccccc56)c5oc6ccccc6c5c4c4c3oc3ccccc34)c3ccccc23)cc1 ; c1ccc(-c2ccccc2-c2c3ccccc3c(-c3cc4cc(-c5c6ccccc6c(-c6ccccc6)c6ccccc56)c5oc6ccccc6c5c4c4c3oc3ccccc34)c3ccccc23)cc1 ; c1ccc(-c2c3ccccc3c(-c3cc4cc(-c5c6ccccc6c(-c6cccc7oc8ccccc8c67)c6ccccc56)c5oc6ccccc6c5c4c4c3oc3ccccc34)c3ccccc23)cc1]

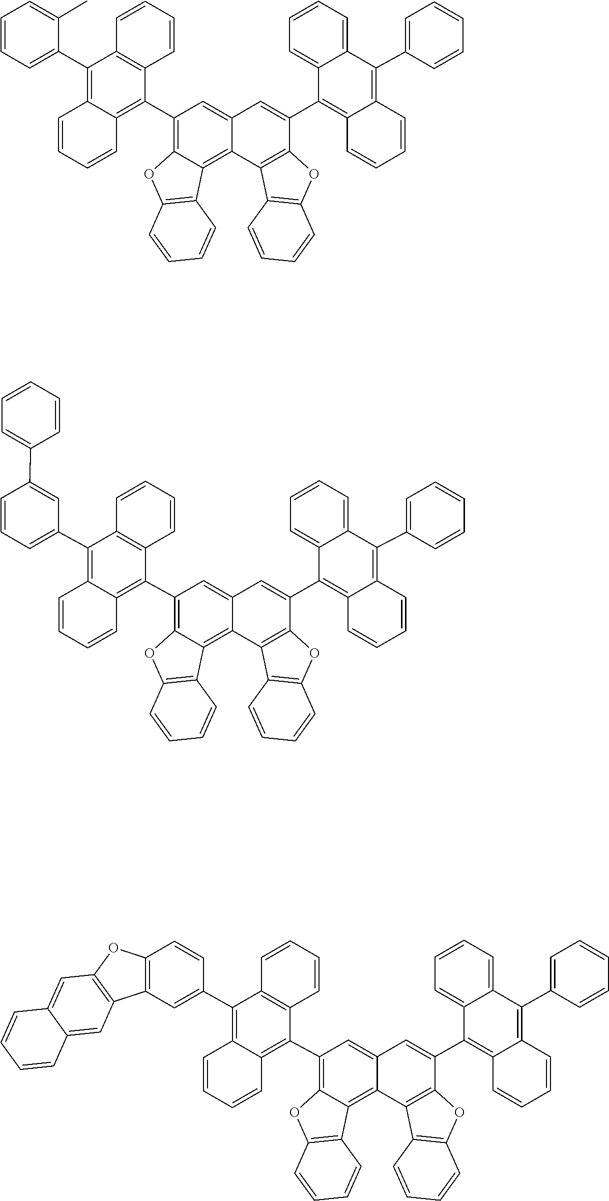 [SMILES: Cc1ccccc1-c1c2ccccc2c(-c2cc3cc(-c4c5ccccc5c(-c5ccccc5)c5ccccc45)c4oc5ccccc5c4c3c3c2oc2ccccc23)c2ccccc12 ; c1ccc(-c2cccc(-c3c4ccccc4c(-c4cc5cc(-c6c7ccccc7c(-c7ccccc7)c7ccccc67)c6oc7ccccc7c6c5c5c4oc4ccccc45)c4ccccc34)c2)cc1 ; c1ccc(-c2c3ccccc3c(-c3cc4cc(-c5c6ccccc6c(-c6ccc7oc8cc9ccccc9cc8c7c6)c6ccccc56)c5oc6ccccc6c5c4c4c3oc3ccccc34)c3ccccc23)cc1]

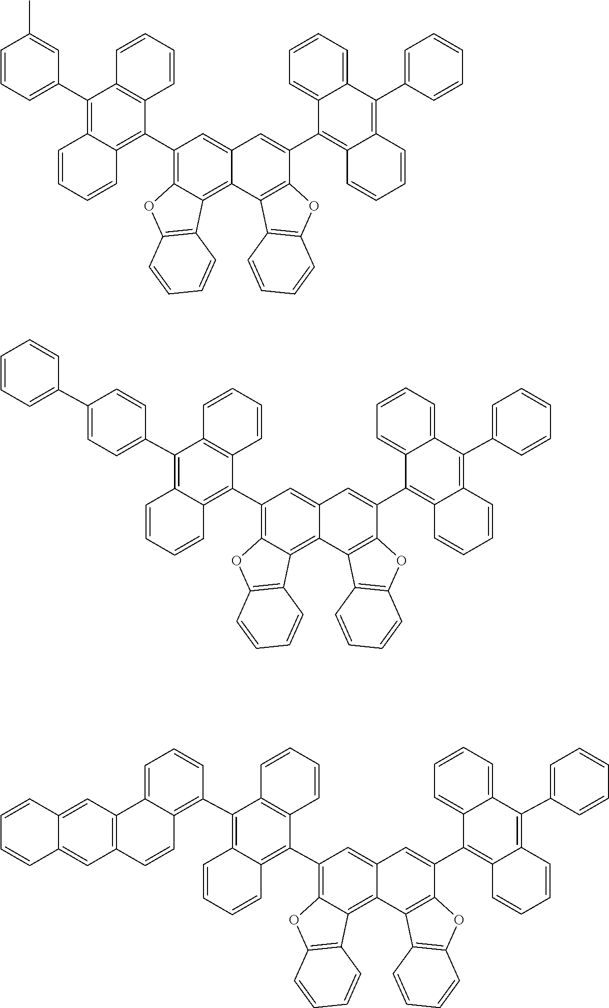 [SMILES: Cc1cccc(-c2c3ccccc3c(-c3cc4cc(-c5c6ccccc6c(-c6ccccc6)c6ccccc56)c5oc6ccccc6c5c4c4c3oc3ccccc34)c3ccccc23)c1 ; c1ccc(-c2ccc(-c3c4ccccc4c(-c4cc5cc(-c6c7ccccc7c(-c7ccccc7)c7ccccc67)c6oc7ccccc7c6c5c5c4oc4ccccc45)c4ccccc34)cc2)cc1 ; c1ccc(-c2c3ccccc3c(-c3cc4cc(-c5c6ccccc6c(-c6cccc7c6ccc6cc8ccccc8cc67)c6ccccc56)c5oc6ccccc6c5c4c4c3oc3ccccc34)c3ccccc23)cc1]

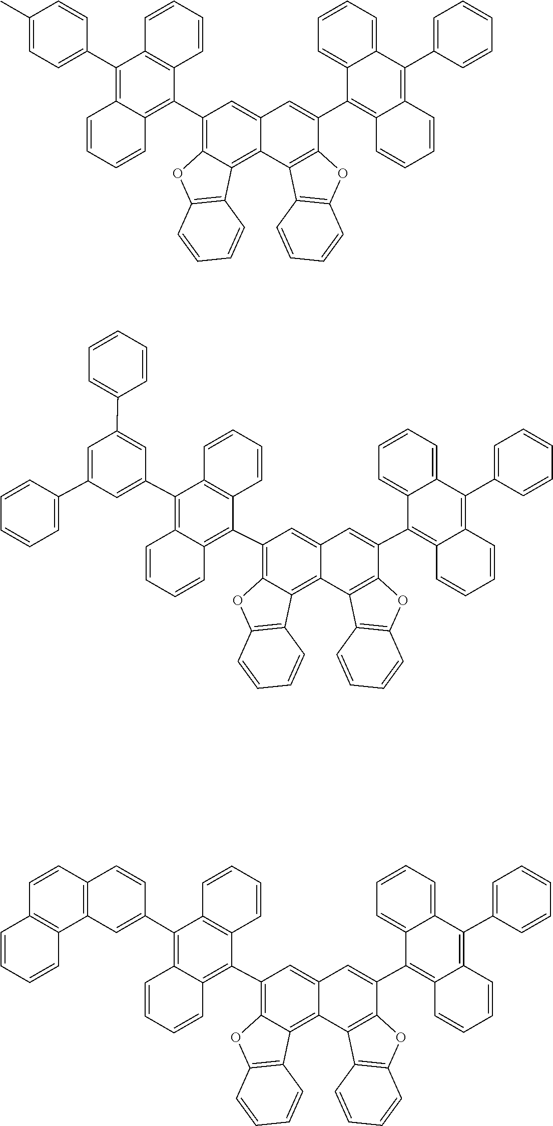 [SMILES: Cc1ccc(-c2c3ccccc3c(-c3cc4cc(-c5c6ccccc6c(-c6ccccc6)c6ccccc56)c5oc6ccccc6c5c4c4c3oc3ccccc34)c3ccccc23)cc1 ; c1ccc(-c2cc(-c3ccccc3)cc(-c3c4ccccc4c(-c4cc5cc(-c6c7ccccc7c(-c7ccccc7)c7ccccc67)c6oc7ccccc7c6c5c5c4oc4ccccc45)c4ccccc34)c2)cc1 ; c1ccc(-c2c3ccccc3c(-c3cc4cc(-c5c6ccccc6c(-c6ccc7ccc8ccccc8c7c6)c6ccccc56)c5oc6ccccc6c5c4c4c3oc3ccccc34)c3ccccc23)cc1]

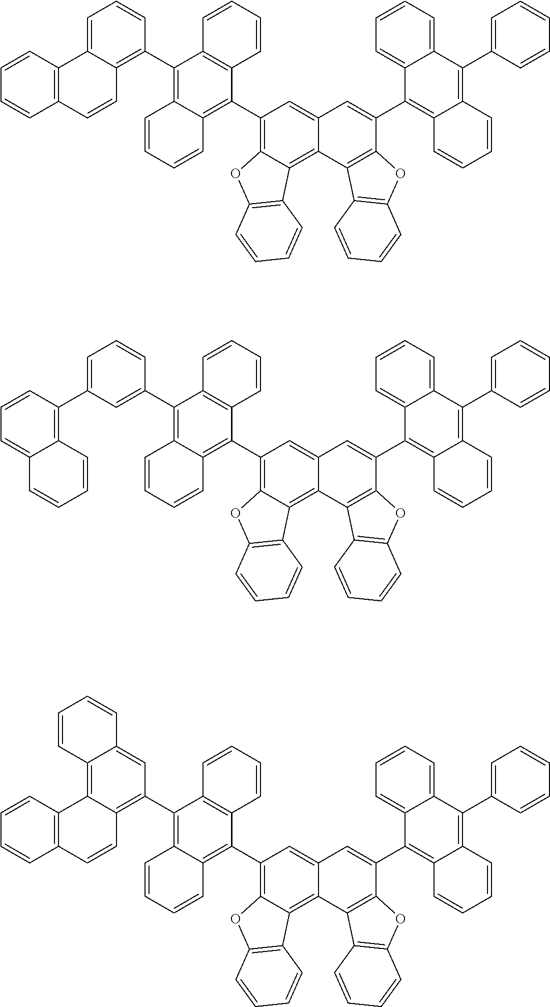 [SMILES: c1ccc(-c2c3ccccc3c(-c3cc4cc(-c5c6ccccc6c(-c6cccc7c6ccc6ccccc67)c6ccccc56)c5oc6ccccc6c5c4c4c3oc3ccccc34)c3ccccc23)cc1 ; c1ccc(-c2c3ccccc3c(-c3cc4cc(-c5c6ccccc6c(-c6cccc(-c7cccc8ccccc78)c6)c6ccccc56)c5oc6ccccc6c5c4c4c3oc3ccccc34)c3ccccc23)cc1 ; c1ccc(-c2c3ccccc3c(-c3cc4cc(-c5c6ccccc6c(-c6cc7ccccc7c7c6ccc6ccccc67)c6ccccc56)c5oc6ccccc6c5c4c4c3oc3ccccc34)c3ccccc23)cc1]

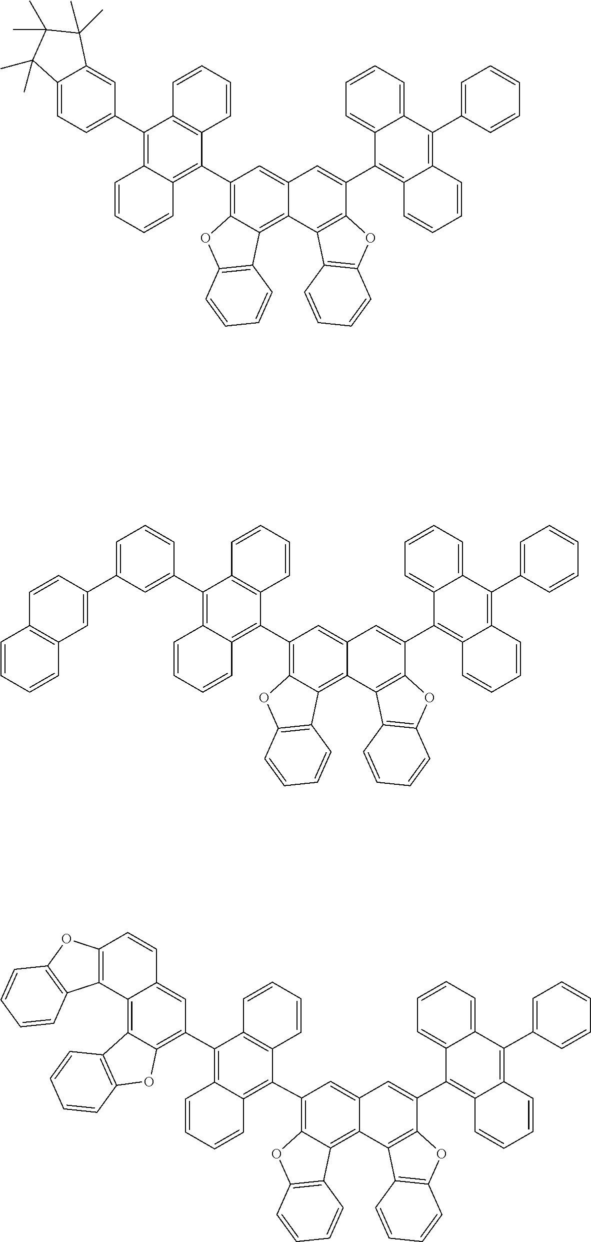 [SMILES: CC1(C)c2ccc(-c3c4ccccc4c(-c4cc5cc(-c6c7ccccc7c(-c7ccccc7)c7ccccc67)c6oc7ccccc7c6c5c5c4oc4ccccc45)c4ccccc34)cc2C(C)(C)C1(C)C ; c1ccc(-c2c3ccccc3c(-c3cc4cc(-c5c6ccccc6c(-c6cccc(-c7ccc8ccccc8c7)c6)c6ccccc56)c5oc6ccccc6c5c4c4c3oc3ccccc34)c3ccccc23)cc1 ; c1ccc(-c2c3ccccc3c(-c3cc4cc(-c5c6ccccc6c(-c6cc7ccc8oc9ccccc9c8c7c7c6oc6ccccc67)c6ccccc56)c5oc6ccccc6c5c4c4c3oc3ccccc34)c3ccccc23)cc1]

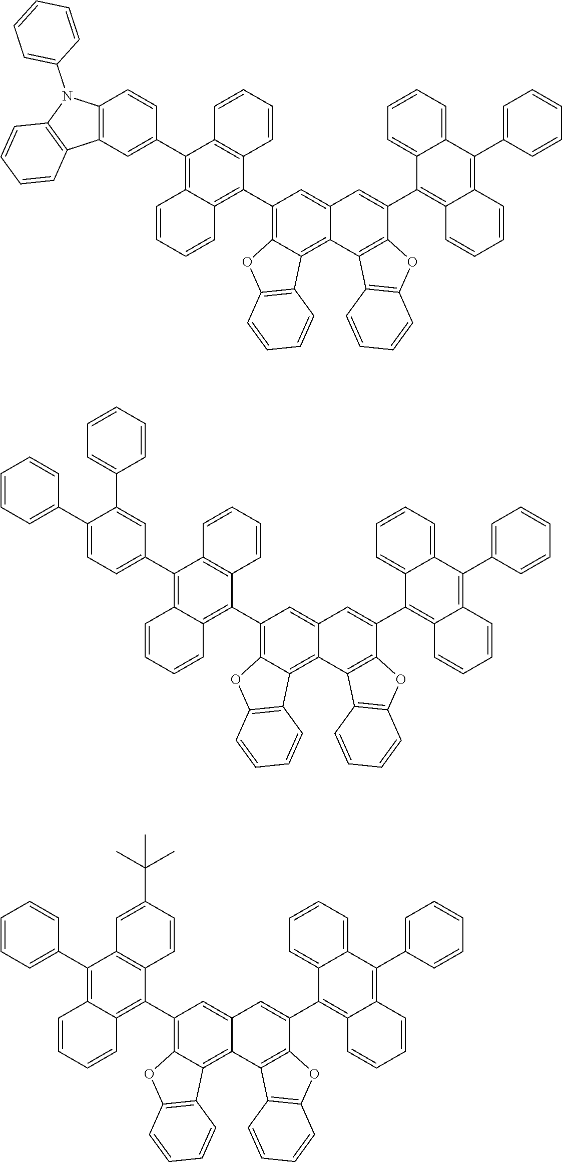 [SMILES: c1ccc(-c2c3ccccc3c(-c3cc4cc(-c5c6ccccc6c(-c6ccc7c(c6)c6ccccc6n7-c6ccccc6)c6ccccc56)c5oc6ccccc6c5c4c4c3oc3ccccc34)c3ccccc23)cc1 ; c1ccc(-c2ccc(-c3c4ccccc4c(-c4cc5cc(-c6c7ccccc7c(-c7ccccc7)c7ccccc67)c6oc7ccccc7c6c5c5c4oc4ccccc45)c4ccccc34)cc2-c2ccccc2)cc1 ; CC(C)(C)c1ccc2c(-c3cc4cc(-c5c6ccccc6c(-c6ccccc6)c6ccccc56)c5oc6ccccc6c5c4c4c3oc3ccccc34)c3ccccc3c(-c3ccccc3)c2c1]

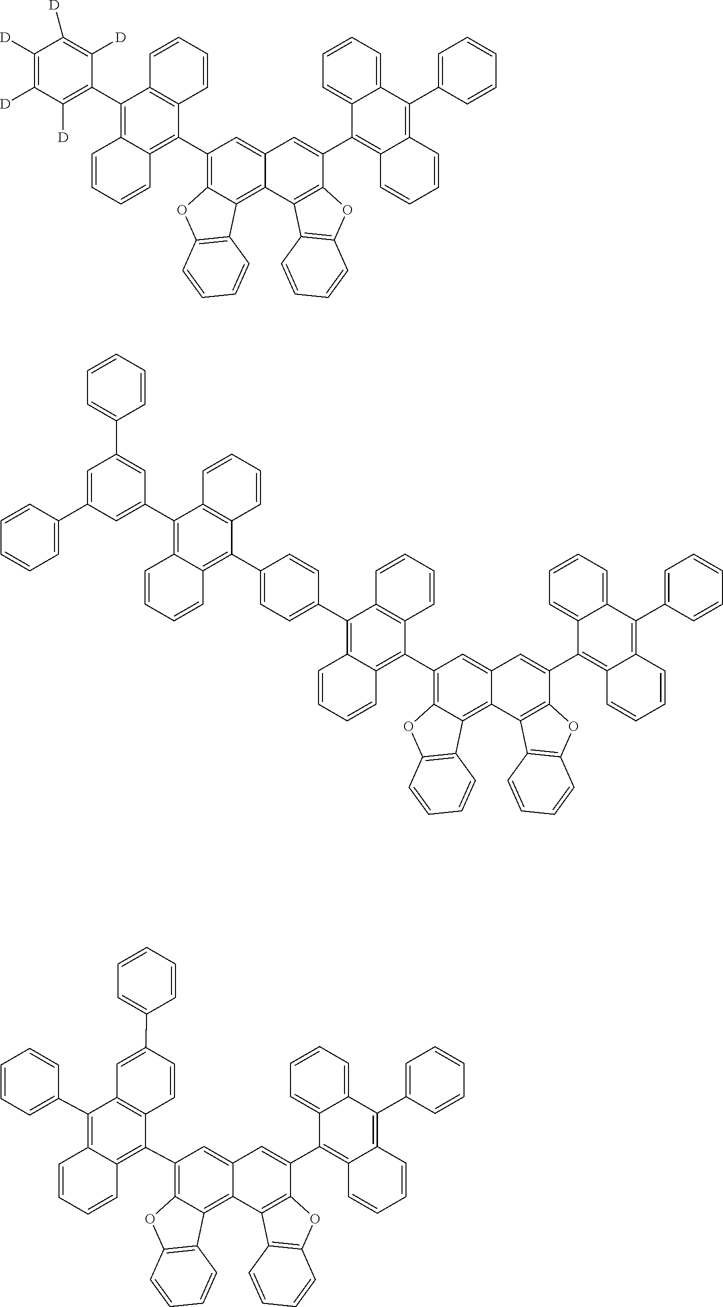 [SMILES: [2H]c1c([2H])c([2H])c(-c2c3ccccc3c(-c3cc4cc(-c5c6ccccc6c(-c6ccccc6)c6ccccc56)c5oc6ccccc6c5c4c4c3oc3ccccc34)c3ccccc23)c([2H])c1[2H] ; c1ccc(-c2cc(-c3ccccc3)cc(-c3c4ccccc4c(-c4ccc(-c5c6ccccc6c(-c6cc7cc(-c8c9ccccc9c(-c9ccccc9)c9ccccc89)c8oc9ccccc9c8c7c7c6oc6ccccc67)c6ccccc56)cc4)c4ccccc34)c2)cc1 ; c1ccc(-c2ccc3c(-c4cc5cc(-c6c7ccccc7c(-c7ccccc7)c7ccccc67)c6oc7ccccc7c6c5c5c4oc4ccccc45)c4ccccc4c(-c4ccccc4)c3c2)cc1]

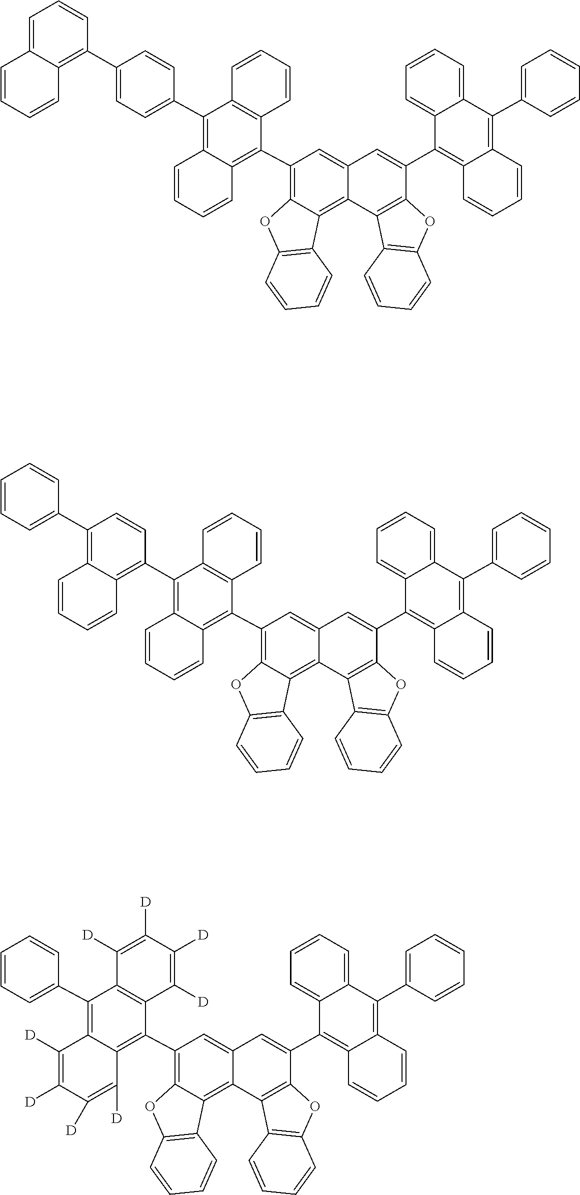 [SMILES: c1ccc(-c2c3ccccc3c(-c3cc4cc(-c5c6ccccc6c(-c6ccc(-c7cccc8ccccc78)cc6)c6ccccc56)c5oc6ccccc6c5c4c4c3oc3ccccc34)c3ccccc23)cc1 ; c1ccc(-c2ccc(-c3c4ccccc4c(-c4cc5cc(-c6c7ccccc7c(-c7ccccc7)c7ccccc67)c6oc7ccccc7c6c5c5c4oc4ccccc45)c4ccccc34)c3ccccc23)cc1 ; [2H]c1c([2H])c([2H])c2c(-c3cc4cc(-c5c6ccccc6c(-c6ccccc6)c6ccccc56)c5oc6ccccc6c5c4c4c3oc3ccccc34)c3c([2H])c([2H])c([2H])c([2H])c3c(-c3ccccc3)c2c1[2H]]

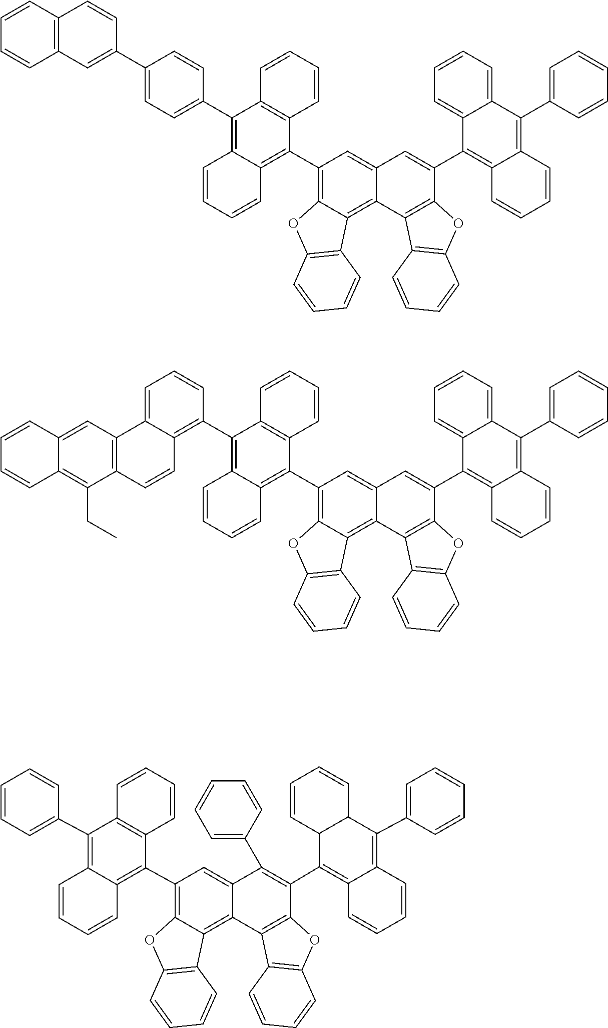 [SMILES: c1ccc(-c2c3ccccc3c(-c3cc4cc(-c5c6ccccc6c(-c6ccc(-c7ccc8ccccc8c7)cc6)c6ccccc56)c5oc6ccccc6c5c4c4c3oc3ccccc34)c3ccccc23)cc1 ; CCc1c2ccccc2cc2c1ccc1c(-c3c4ccccc4c(-c4cc5cc(-c6c7ccccc7c(-c7ccccc7)c7ccccc67)c6oc7ccccc7c6c5c5c4oc4ccccc45)c4ccccc34)cccc12 ; C1=CC2C(c3ccccc3)=c3ccccc3=C(c3c(-c4ccccc4)c4cc(-c5c6ccccc6c(-c6ccccc6)c6ccccc56)c5oc6ccccc6c5c4c4c3oc3ccccc34)C2C=C1]

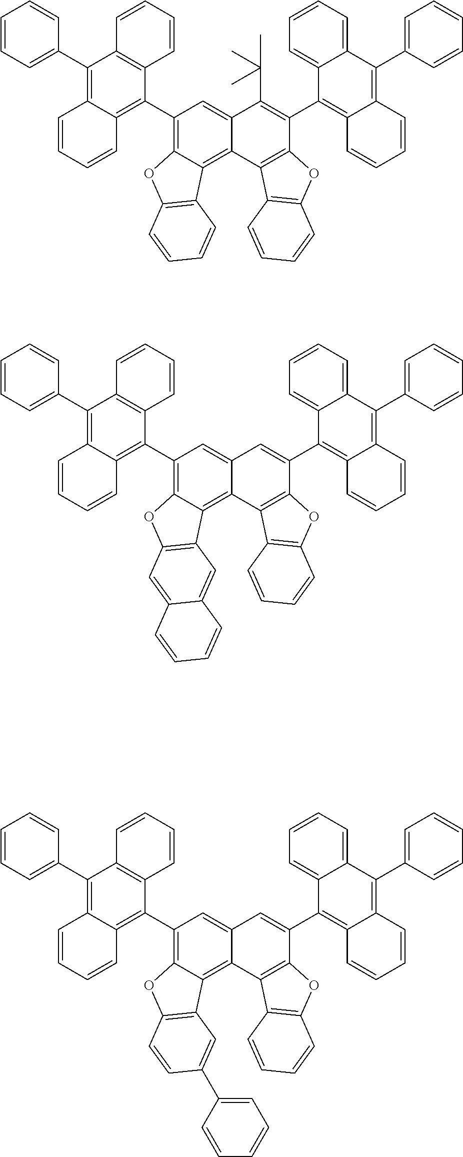 [SMILES: CC(C)(C)c1c(-c2c3ccccc3c(-c3ccccc3)c3ccccc23)c2oc3ccccc3c2c2c1cc(-c1c3ccccc3c(-c3ccccc3)c3ccccc13)c1oc3ccccc3c12 ; c1ccc(-c2c3ccccc3c(-c3cc4cc(-c5c6ccccc6c(-c6ccccc6)c6ccccc56)c5oc6cc7ccccc7cc6c5c4c4c3oc3ccccc34)c3ccccc23)cc1 ; c1ccc(-c2ccc3oc4c(-c5c6ccccc6c(-c6ccccc6)c6ccccc56)cc5cc(-c6c7ccccc7c(-c7ccccc7)c7ccccc67)c6oc7ccccc7c6c5c4c3c2)cc1]

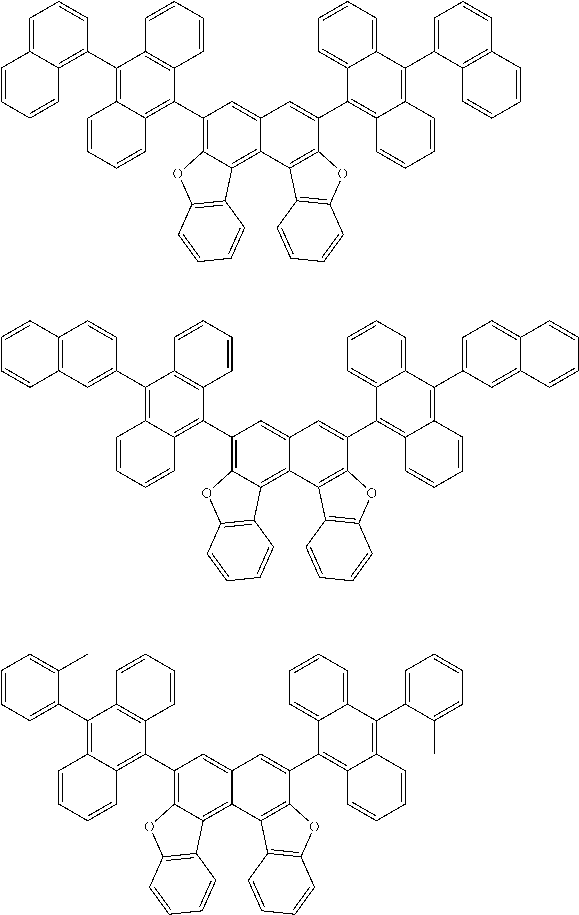 [SMILES: c1ccc2c(-c3c4ccccc4c(-c4cc5cc(-c6c7ccccc7c(-c7cccc8ccccc78)c7ccccc67)c6oc7ccccc7c6c5c5c4oc4ccccc45)c4ccccc34)cccc2c1 ; c1ccc2cc(-c3c4ccccc4c(-c4cc5cc(-c6c7ccccc7c(-c7ccc8ccccc8c7)c7ccccc67)c6oc7ccccc7c6c5c5c4oc4ccccc45)c4ccccc34)ccc2c1 ; Cc1ccccc1-c1c2ccccc2c(-c2cc3cc(-c4c5ccccc5c(-c5ccccc5C)c5ccccc45)c4oc5ccccc5c4c3c3c2oc2ccccc23)c2ccccc12]

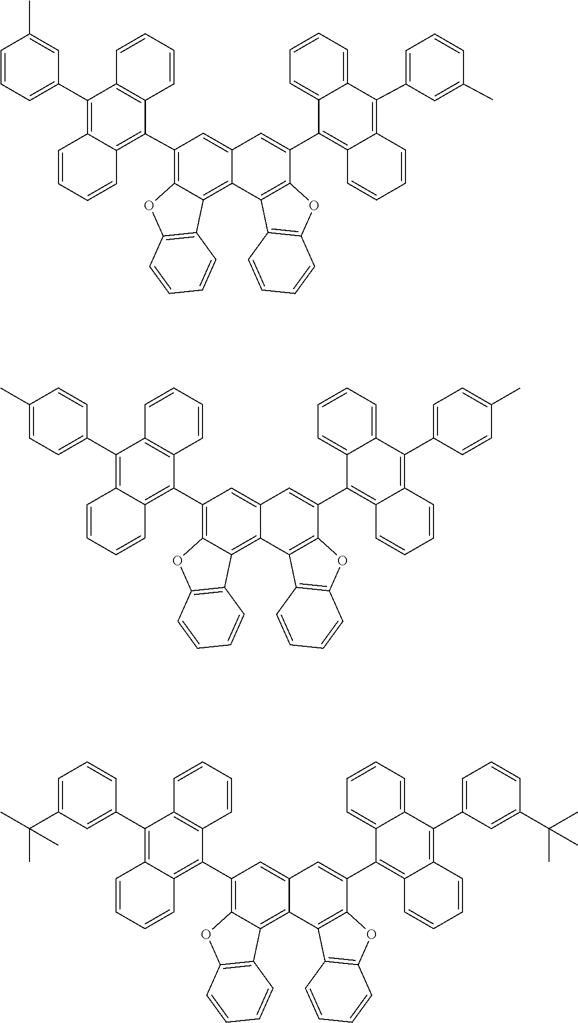

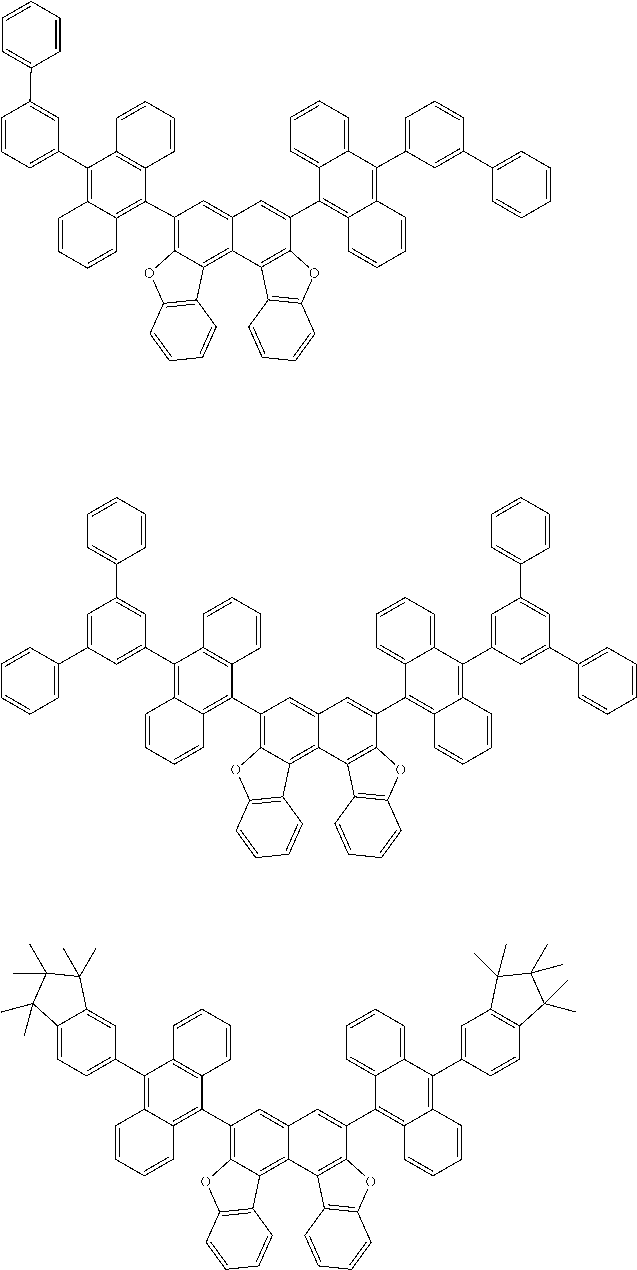

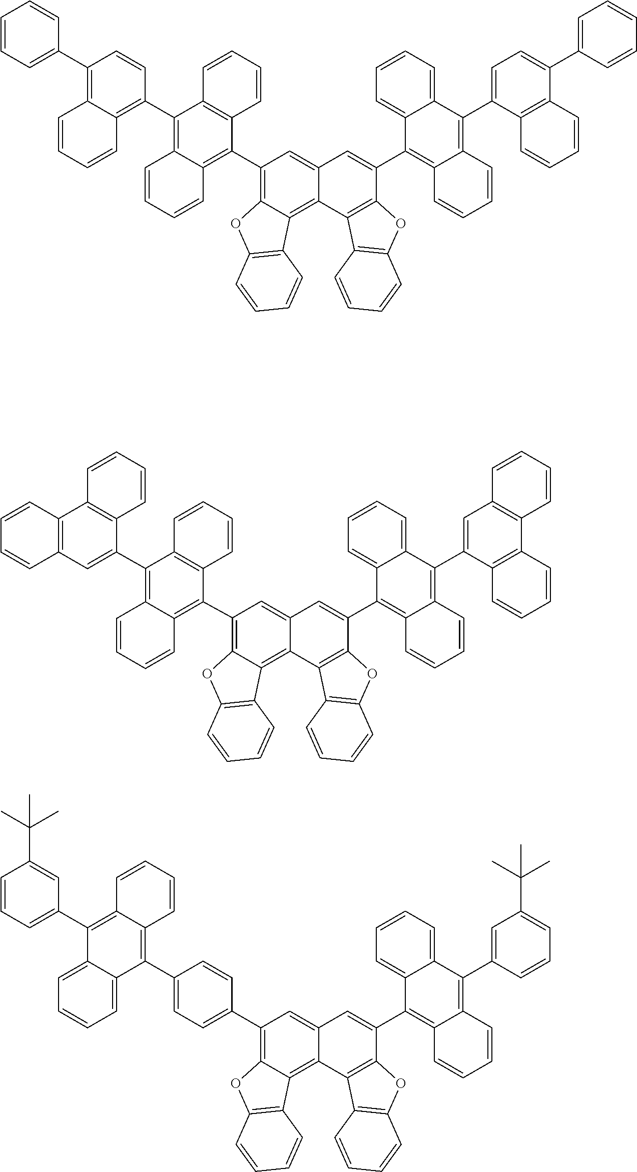 [SMILES: c1ccc(-c2ccc(-c3c4ccccc4c(-c4cc5cc(-c6c7ccccc7c(-c7ccc(-c8ccccc8)c8ccccc78)c7ccccc67)c6oc7ccccc7c6c5c5c4oc4ccccc45)c4ccccc34)c3ccccc23)cc1 ; c1ccc2c(c1)cc(-c1c3ccccc3c(-c3cc4cc(-c5c6ccccc6c(-c6cc7ccccc7c7ccccc67)c6ccccc56)c5oc6ccccc6c5c4c4c3oc3ccccc34)c3ccccc13)c1ccccc12 ; CC(C)(C)c1cccc(-c2c3ccccc3c(-c3ccc(-c4cc5cc(-c6c7ccccc7c(-c7cccc(C(C)(C)C)c7)c7ccccc67)c6oc7ccccc7c6c5c5c4oc4ccccc45)cc3)c3ccccc23)c1]

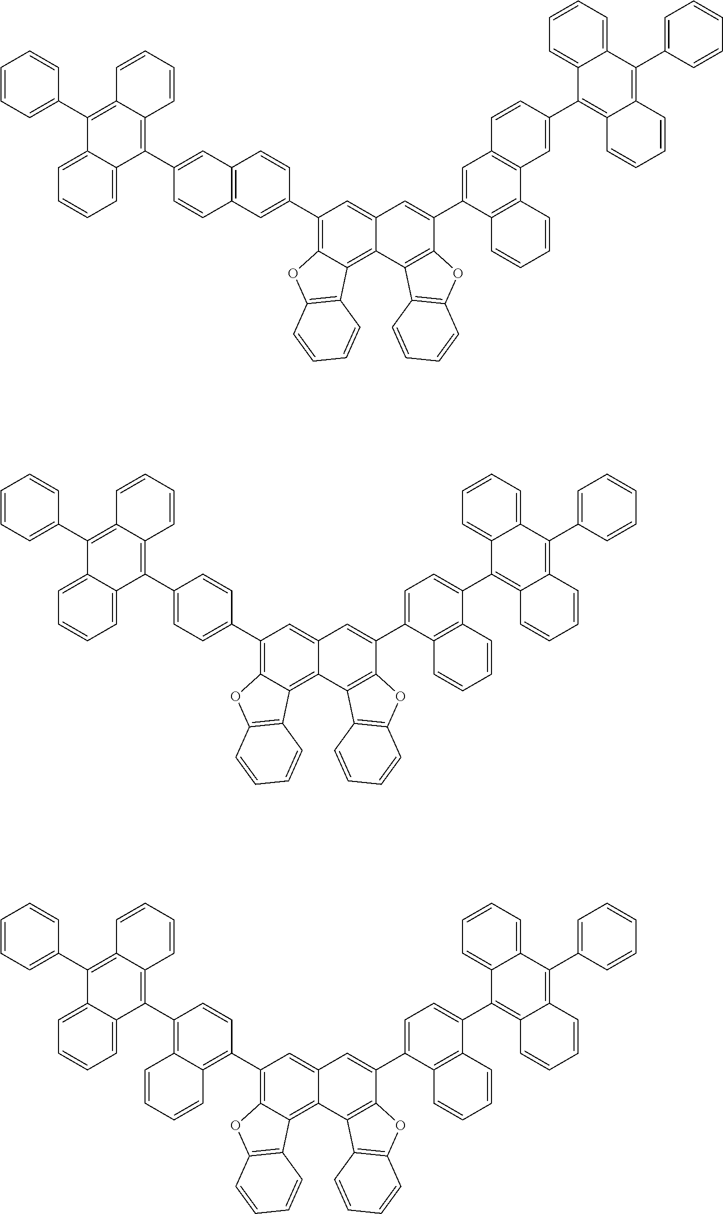 [SMILES: c1ccc(-c2c3ccccc3c(-c3ccc4cc(-c5cc6cc(-c7cc8ccc(-c9c%10ccccc%10c(-c%10ccccc%10)c%10ccccc9%10)cc8c8ccccc78)c7oc8ccccc8c7c6c6c5oc5ccccc56)ccc4c3)c3ccccc23)cc1 ; c1ccc(-c2c3ccccc3c(-c3ccc(-c4cc5cc(-c6ccc(-c7c8ccccc8c(-c8ccccc8)c8ccccc78)c7ccccc67)c6oc7ccccc7c6c5c5c4oc4ccccc45)cc3)c3ccccc23)cc1 ; c1ccc(-c2c3ccccc3c(-c3ccc(-c4cc5cc(-c6ccc(-c7c8ccccc8c(-c8ccccc8)c8ccccc78)c7ccccc67)c6oc7ccccc7c6c5c5c4oc4ccccc45)c4ccccc34)c3ccccc23)cc1]

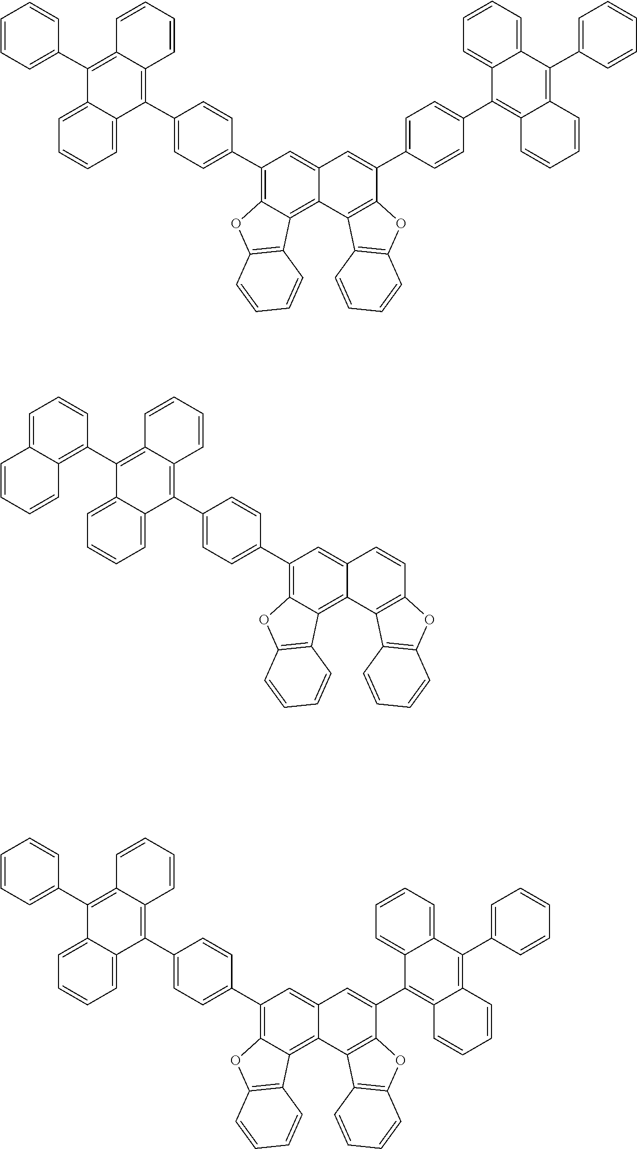 [SMILES: c1ccc(-c2c3ccccc3c(-c3ccc(-c4cc5cc(-c6ccc(-c7c8ccccc8c(-c8ccccc8)c8ccccc78)cc6)c6oc7ccccc7c6c5c5c4oc4ccccc45)cc3)c3ccccc23)cc1 ; c1ccc2c(-c3c4ccccc4c(-c4ccc(-c5cc6ccc7oc8ccccc8c7c6c6c5oc5ccccc56)cc4)c4ccccc34)cccc2c1 ; c1ccc(-c2c3ccccc3c(-c3ccc(-c4cc5cc(-c6c7ccccc7c(-c7ccccc7)c7ccccc67)c6oc7ccccc7c6c5c5c4oc4ccccc45)cc3)c3ccccc23)cc1]

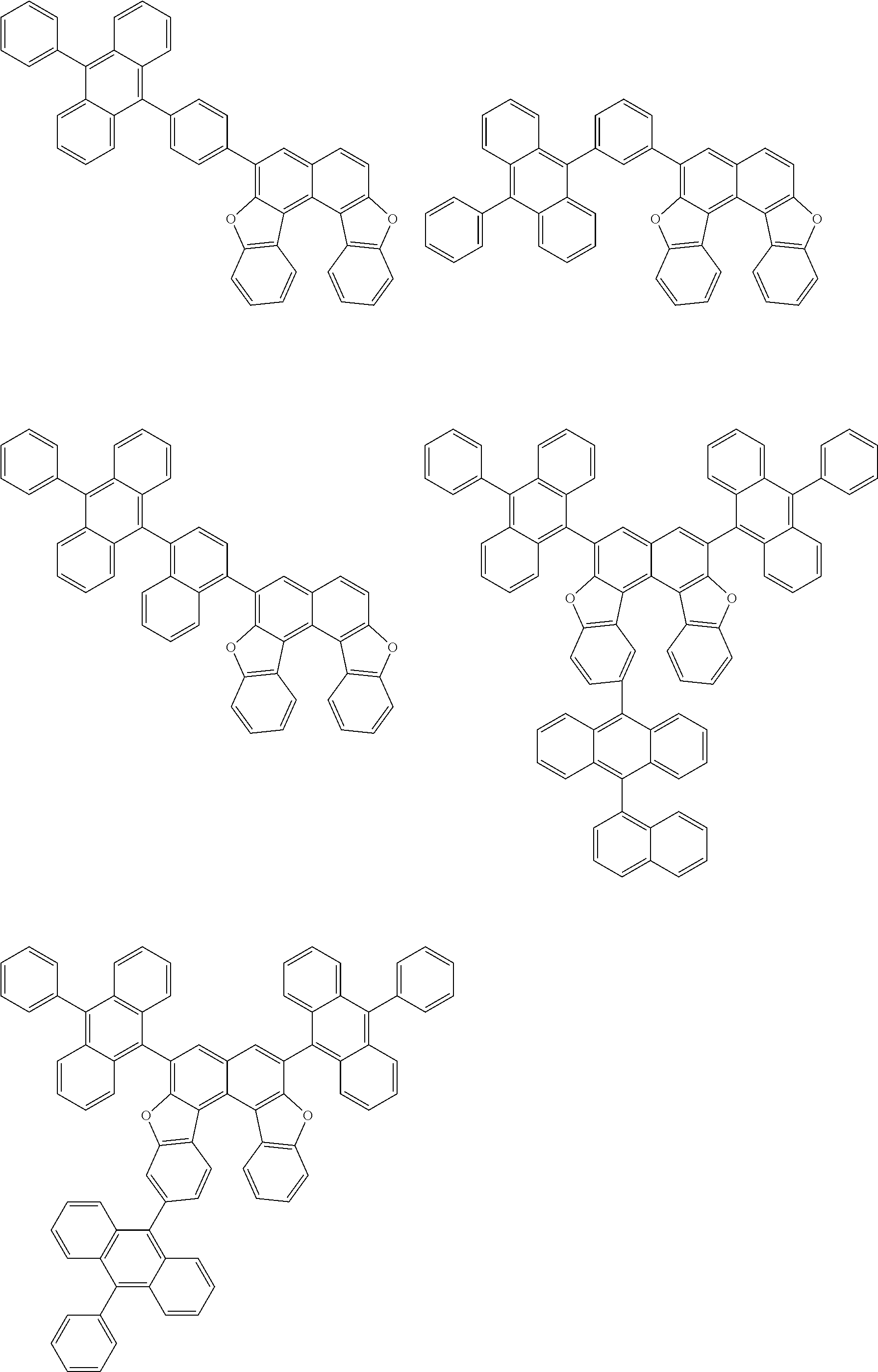

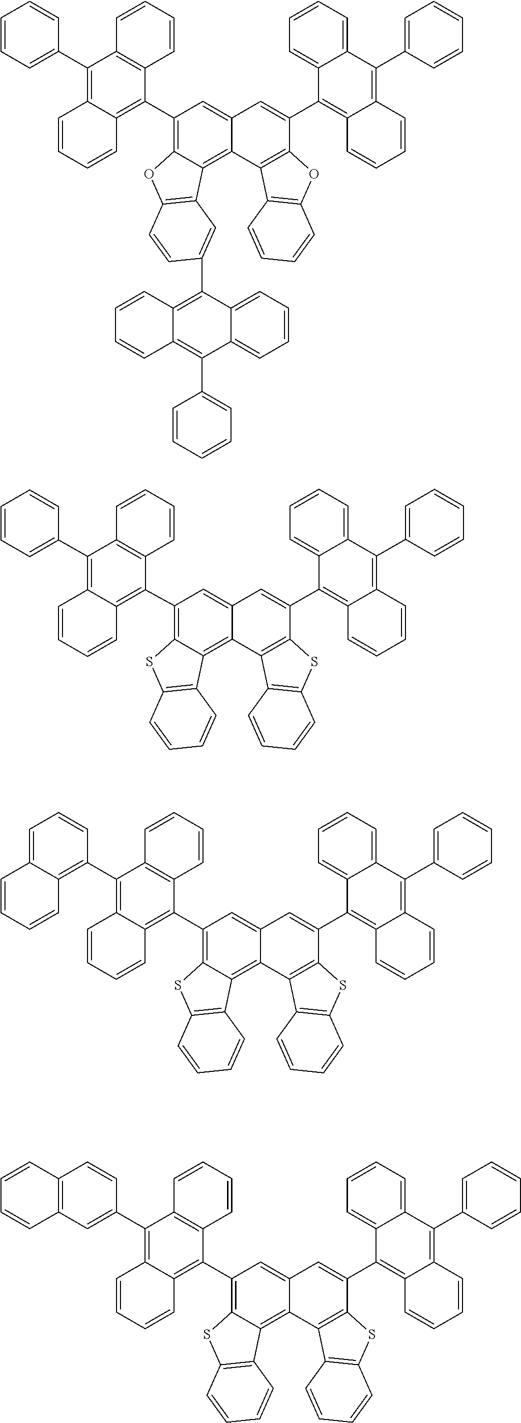 [SMILES: c1ccc(-c2c3ccccc3c(-c3ccc4oc5c(-c6c7ccccc7c(-c7ccccc7)c7ccccc67)cc6cc(-c7c8ccccc8c(-c8ccccc8)c8ccccc78)c7oc8ccccc8c7c6c5c4c3)c3ccccc23)cc1 ; c1ccc(-c2c3ccccc3c(-c3cc4cc(-c5c6ccccc6c(-c6ccccc6)c6ccccc56)c5sc6ccccc6c5c4c4c3sc3ccccc34)c3ccccc23)cc1 ; c1ccc(-c2c3ccccc3c(-c3cc4cc(-c5c6ccccc6c(-c6cccc7ccccc67)c6ccccc56)c5sc6ccccc6c5c4c4c3sc3ccccc34)c3ccccc23)cc1 ; c1ccc(-c2c3ccccc3c(-c3cc4cc(-c5c6ccccc6c(-c6ccc7ccccc7c6)c6ccccc56)c5sc6ccccc6c5c4c4c3sc3ccccc34)c3ccccc23)cc1]

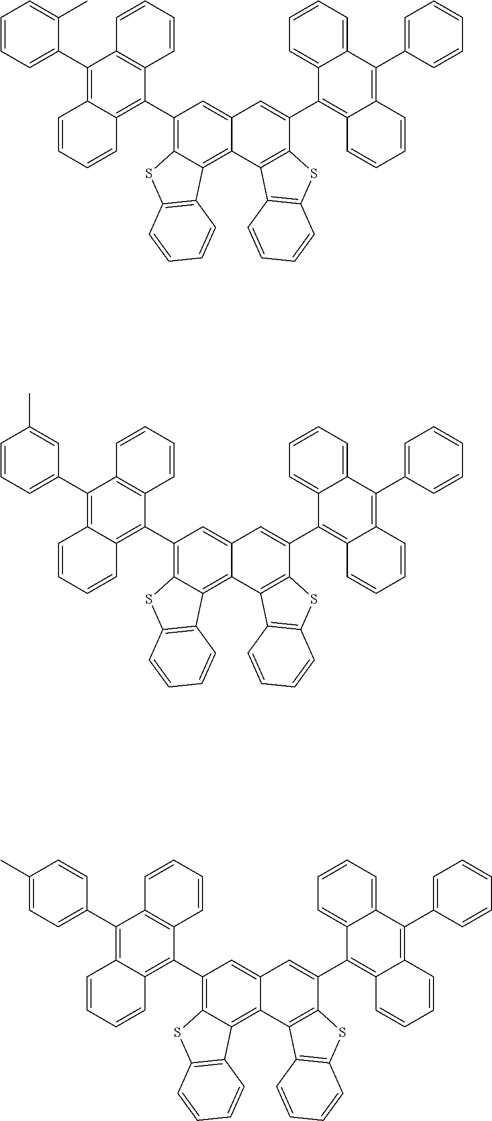 [SMILES: Cc1ccccc1-c1c2ccccc2c(-c2cc3cc(-c4c5ccccc5c(-c5ccccc5)c5ccccc45)c4sc5ccccc5c4c3c3c2sc2ccccc23)c2ccccc12 ; Cc1cccc(-c2c3ccccc3c(-c3cc4cc(-c5c6ccccc6c(-c6ccccc6)c6ccccc56)c5sc6ccccc6c5c4c4c3sc3ccccc34)c3ccccc23)c1 ; Cc1ccc(-c2c3ccccc3c(-c3cc4cc(-c5c6ccccc6c(-c6ccccc6)c6ccccc56)c5sc6ccccc6c5c4c4c3sc3ccccc34)c3ccccc23)cc1]

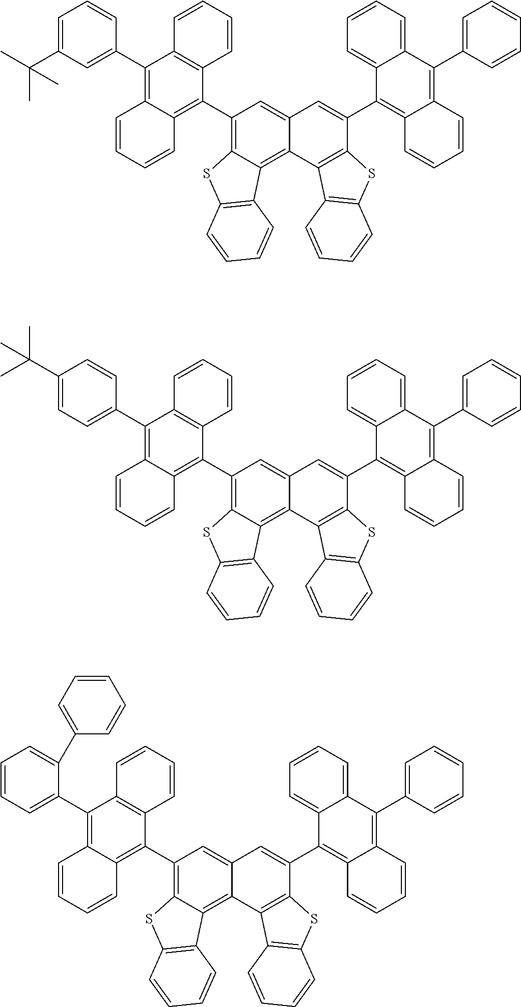 [SMILES: CC(C)(C)c1cccc(-c2c3ccccc3c(-c3cc4cc(-c5c6ccccc6c(-c6ccccc6)c6ccccc56)c5sc6ccccc6c5c4c4c3sc3ccccc34)c3ccccc23)c1 ; CC(C)(C)c1ccc(-c2c3ccccc3c(-c3cc4cc(-c5c6ccccc6c(-c6ccccc6)c6ccccc56)c5sc6ccccc6c5c4c4c3sc3ccccc34)c3ccccc23)cc1 ; c1ccc(-c2ccccc2-c2c3ccccc3c(-c3cc4cc(-c5c6ccccc6c(-c6ccccc6)c6ccccc56)c5sc6ccccc6c5c4c4c3sc3ccccc34)c3ccccc23)cc1]

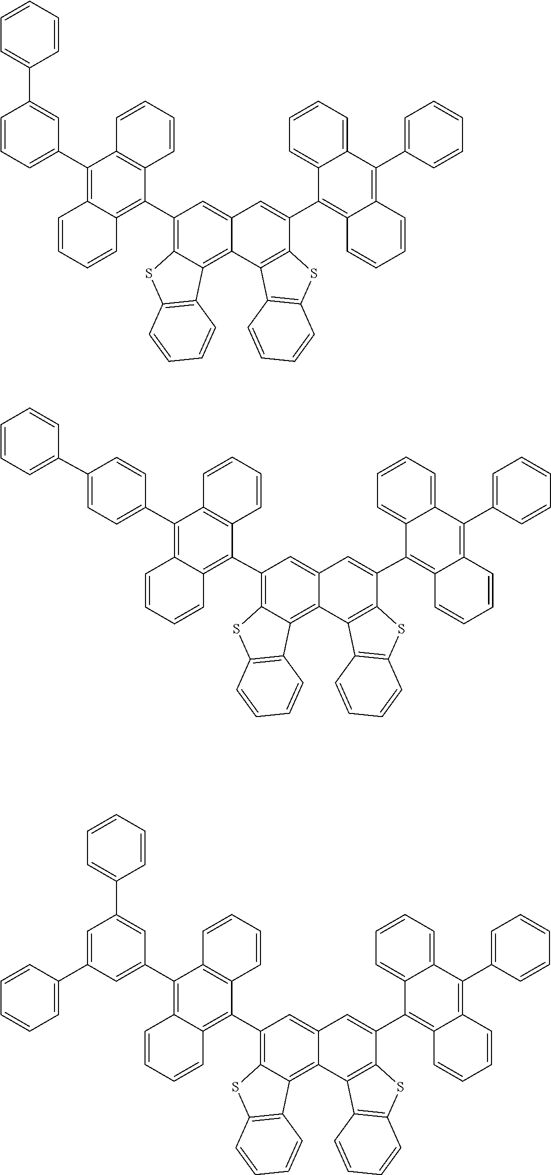 [SMILES: c1ccc(-c2cccc(-c3c4ccccc4c(-c4cc5cc(-c6c7ccccc7c(-c7ccccc7)c7ccccc67)c6sc7ccccc7c6c5c5c4sc4ccccc45)c4ccccc34)c2)cc1 ; c1ccc(-c2ccc(-c3c4ccccc4c(-c4cc5cc(-c6c7ccccc7c(-c7ccccc7)c7ccccc67)c6sc7ccccc7c6c5c5c4sc4ccccc45)c4ccccc34)cc2)cc1 ; c1ccc(-c2cc(-c3ccccc3)cc(-c3c4ccccc4c(-c4cc5cc(-c6c7ccccc7c(-c7ccccc7)c7ccccc67)c6sc7ccccc7c6c5c5c4sc4ccccc45)c4ccccc34)c2)cc1]

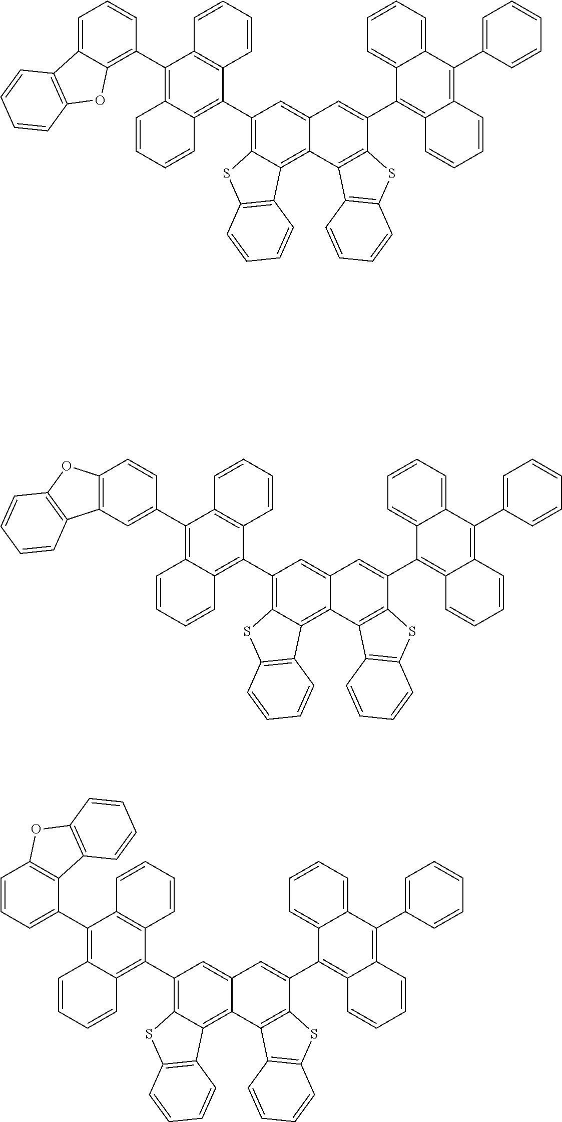 [SMILES: c1ccc(-c2c3ccccc3c(-c3cc4cc(-c5c6ccccc6c(-c6cccc7c6oc6ccccc67)c6ccccc56)c5sc6ccccc6c5c4c4c3sc3ccccc34)c3ccccc23)cc1 ; c1ccc(-c2c3ccccc3c(-c3cc4cc(-c5c6ccccc6c(-c6ccc7oc8ccccc8c7c6)c6ccccc56)c5sc6ccccc6c5c4c4c3sc3ccccc34)c3ccccc23)cc1 ; c1ccc(-c2c3ccccc3c(-c3cc4cc(-c5c6ccccc6c(-c6cccc7oc8ccccc8c67)c6ccccc56)c5sc6ccccc6c5c4c4c3sc3ccccc34)c3ccccc23)cc1]

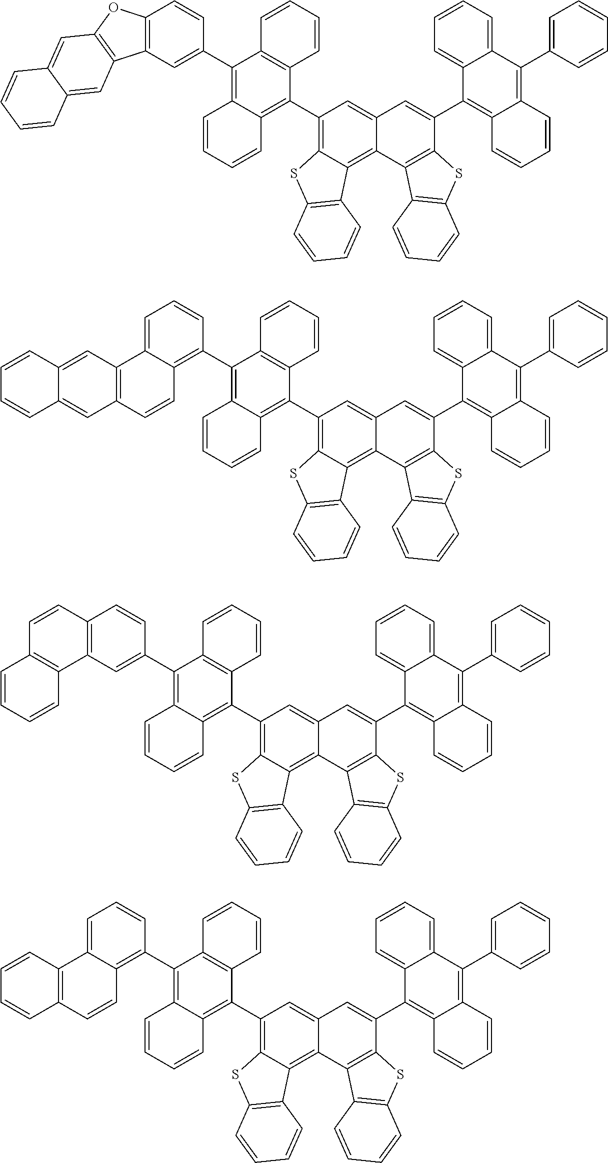

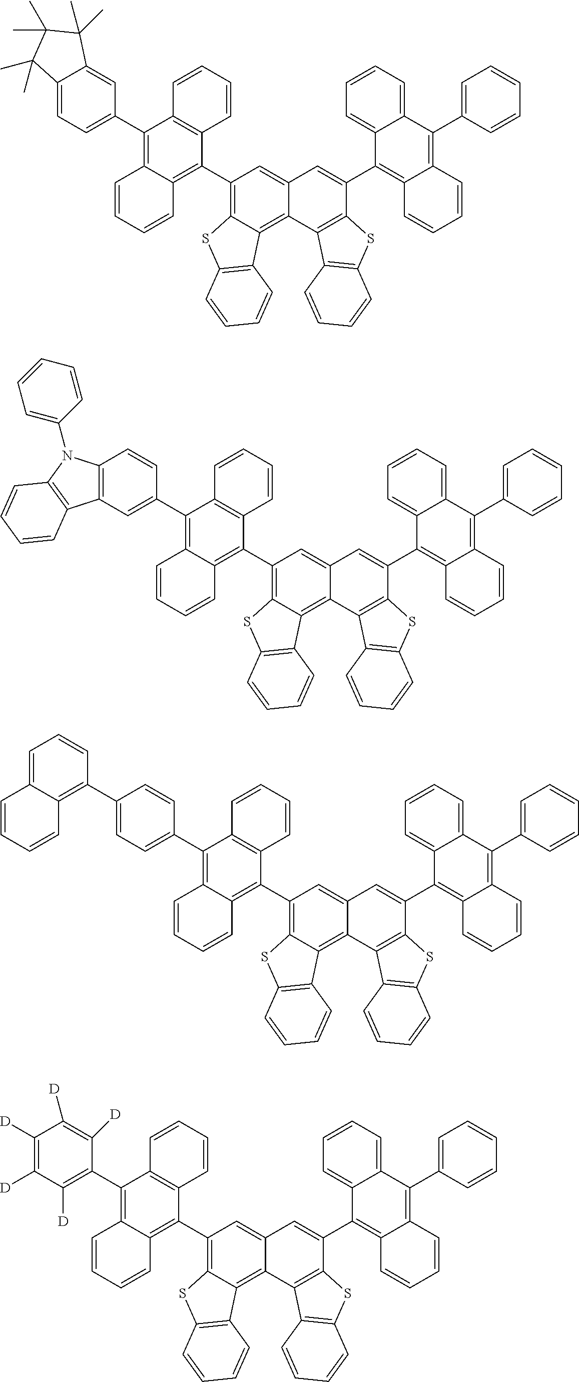

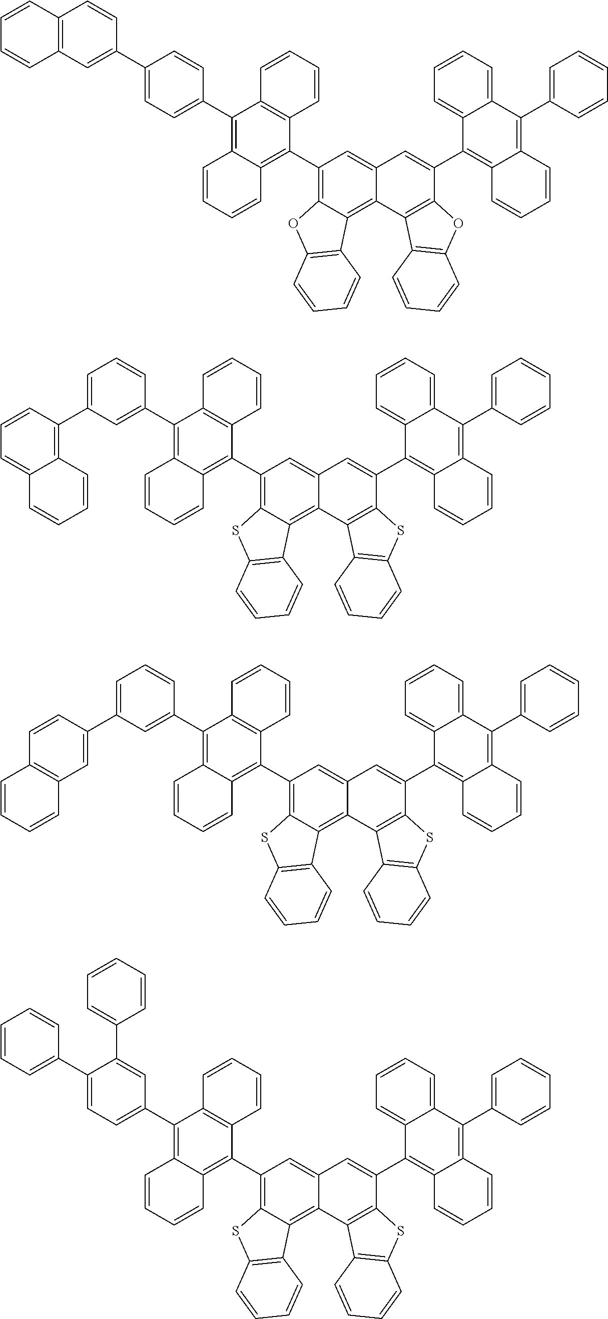 [SMILES: c1ccc(-c2c3ccccc3c(-c3cc4cc(-c5c6ccccc6c(-c6ccc(-c7ccc8ccccc8c7)cc6)c6ccccc56)c5oc6ccccc6c5c4c4c3oc3ccccc34)c3ccccc23)cc1 ; c1ccc(-c2c3ccccc3c(-c3cc4cc(-c5c6ccccc6c(-c6cccc(-c7cccc8ccccc78)c6)c6ccccc56)c5sc6ccccc6c5c4c4c3sc3ccccc34)c3ccccc23)cc1 ; c1ccc(-c2c3ccccc3c(-c3cc4cc(-c5c6ccccc6c(-c6cccc(-c7ccc8ccccc8c7)c6)c6ccccc56)c5sc6ccccc6c5c4c4c3sc3ccccc34)c3ccccc23)cc1 ; c1ccc(-c2ccc(-c3c4ccccc4c(-c4cc5cc(-c6c7ccccc7c(-c7ccccc7)c7ccccc67)c6sc7ccccc7c6c5c5c4sc4ccccc45)c4ccccc34)cc2-c2ccccc2)cc1]

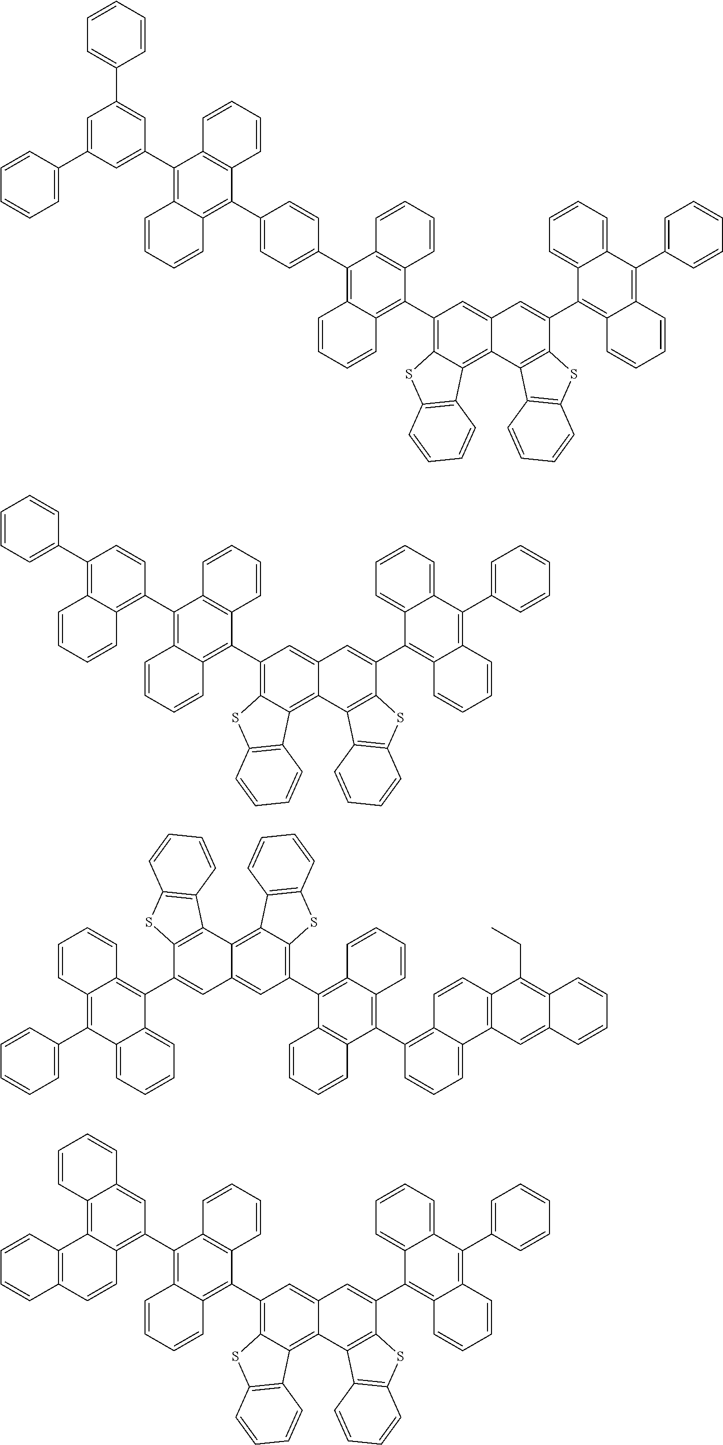

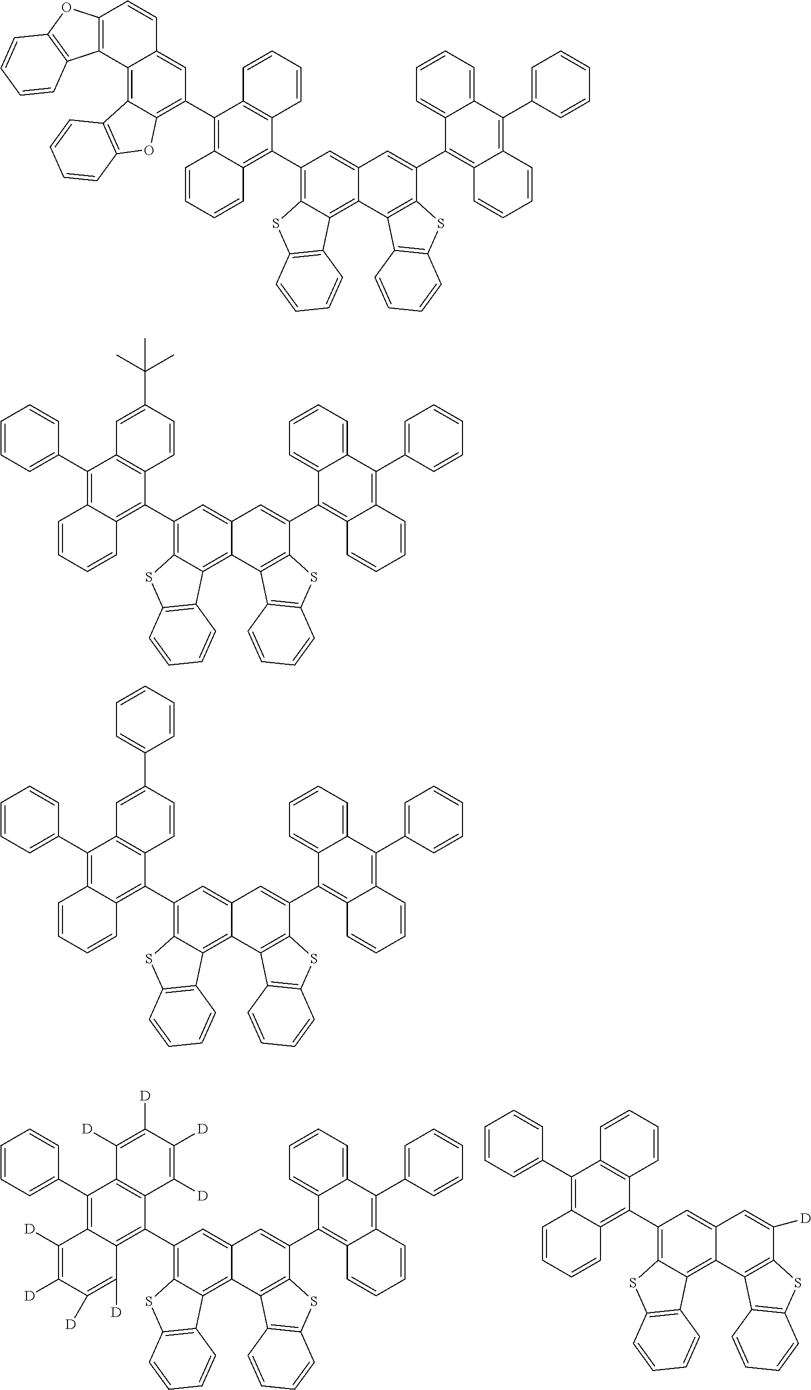

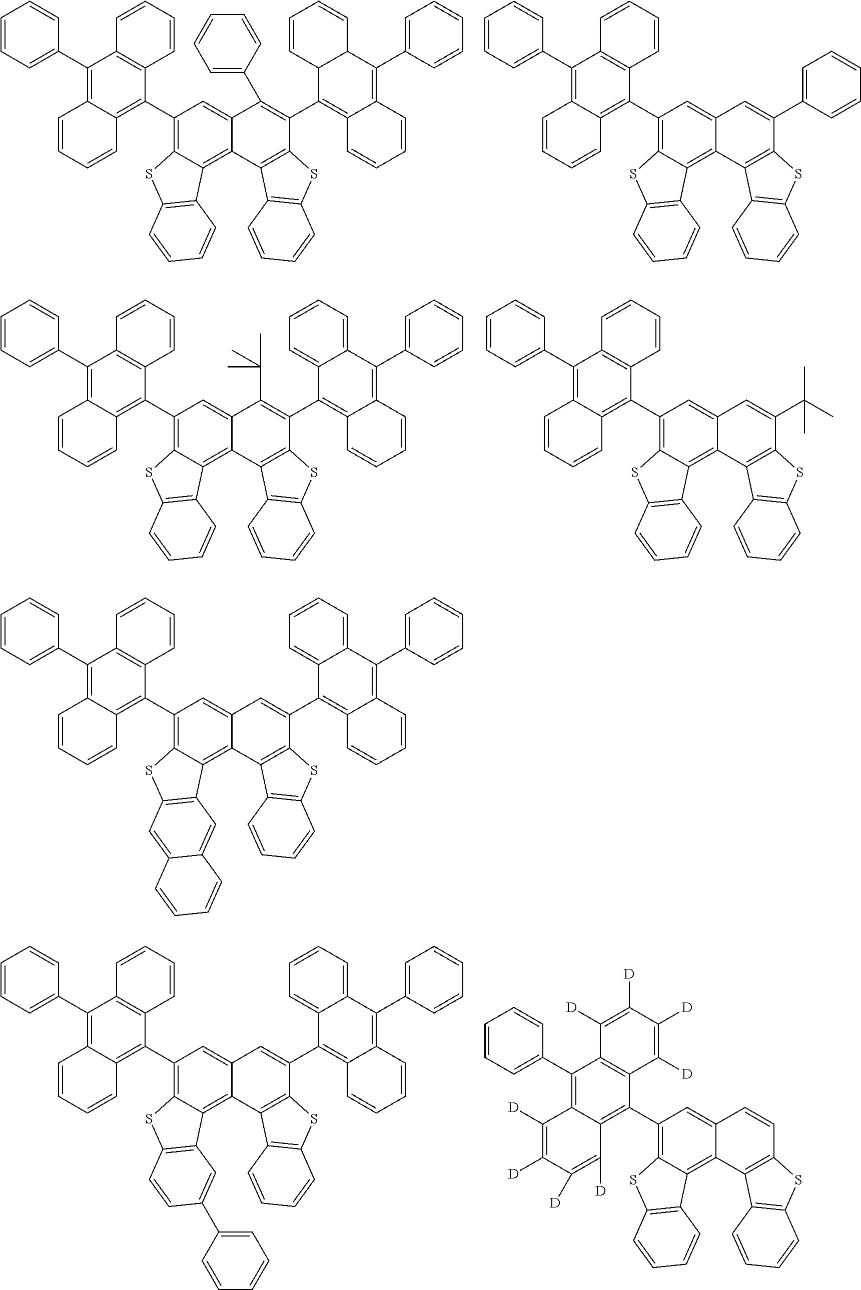

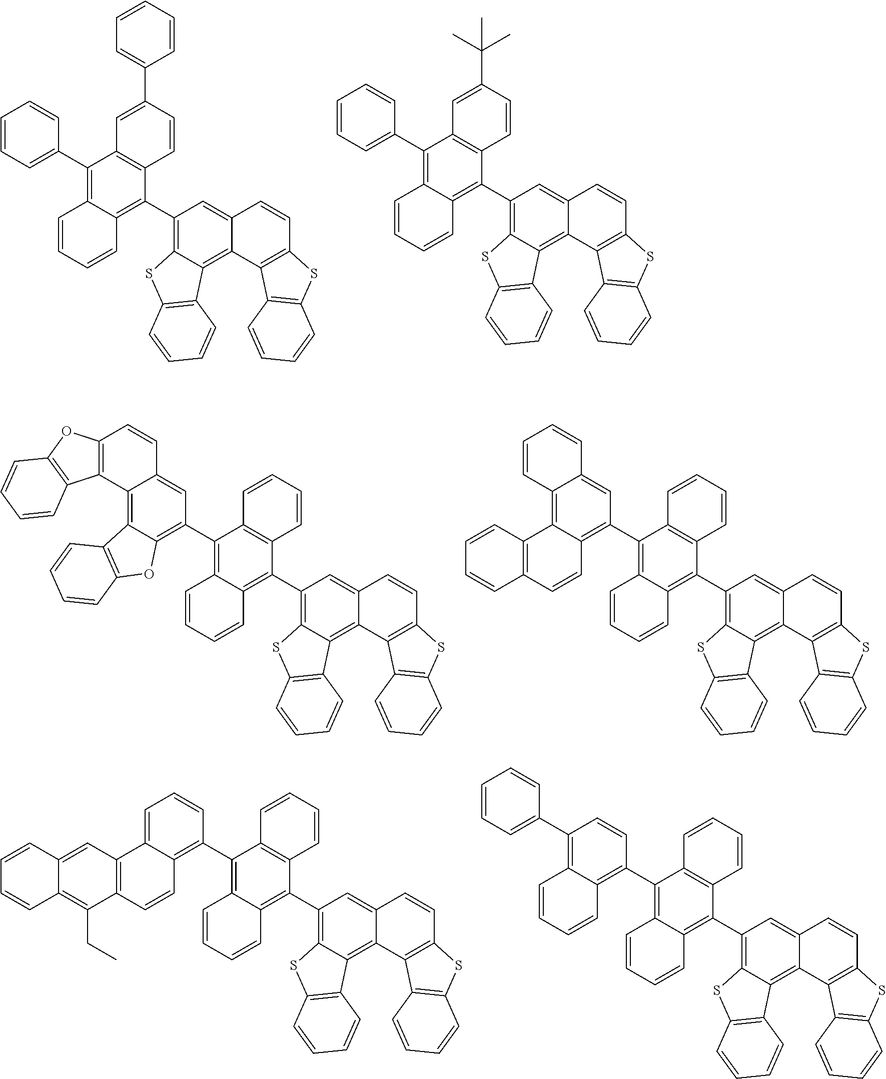

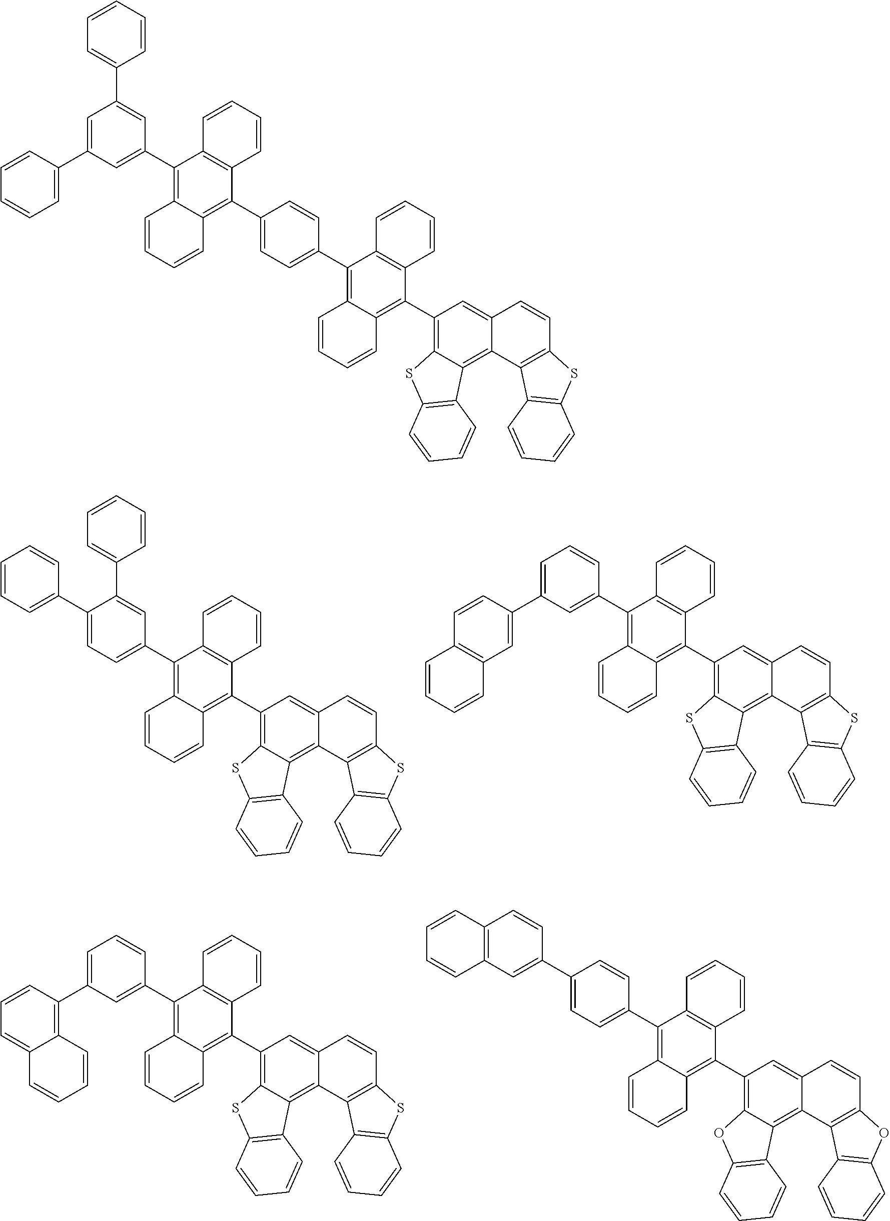 [SMILES: c1ccc(-c2cc(-c3ccccc3)cc(-c3c4ccccc4c(-c4ccc(-c5c6ccccc6c(-c6cc7ccc8sc9ccccc9c8c7c7c6sc6ccccc67)c6ccccc56)cc4)c4ccccc34)c2)cc1 ; c1ccc(-c2ccc(-c3c4ccccc4c(-c4cc5ccc6sc7ccccc7c6c5c5c4sc4ccccc45)c4ccccc34)cc2-c2ccccc2)cc1 ; c1cc(-c2ccc3ccccc3c2)cc(-c2c3ccccc3c(-c3cc4ccc5sc6ccccc6c5c4c4c3sc3ccccc34)c3ccccc23)c1 ; c1cc(-c2cccc3ccccc23)cc(-c2c3ccccc3c(-c3cc4ccc5sc6ccccc6c5c4c4c3sc3ccccc34)c3ccccc23)c1 ; c1ccc2cc(-c3ccc(-c4c5ccccc5c(-c5cc6ccc7oc8ccccc8c7c6c6c5oc5ccccc56)c5ccccc45)cc3)ccc2c1]

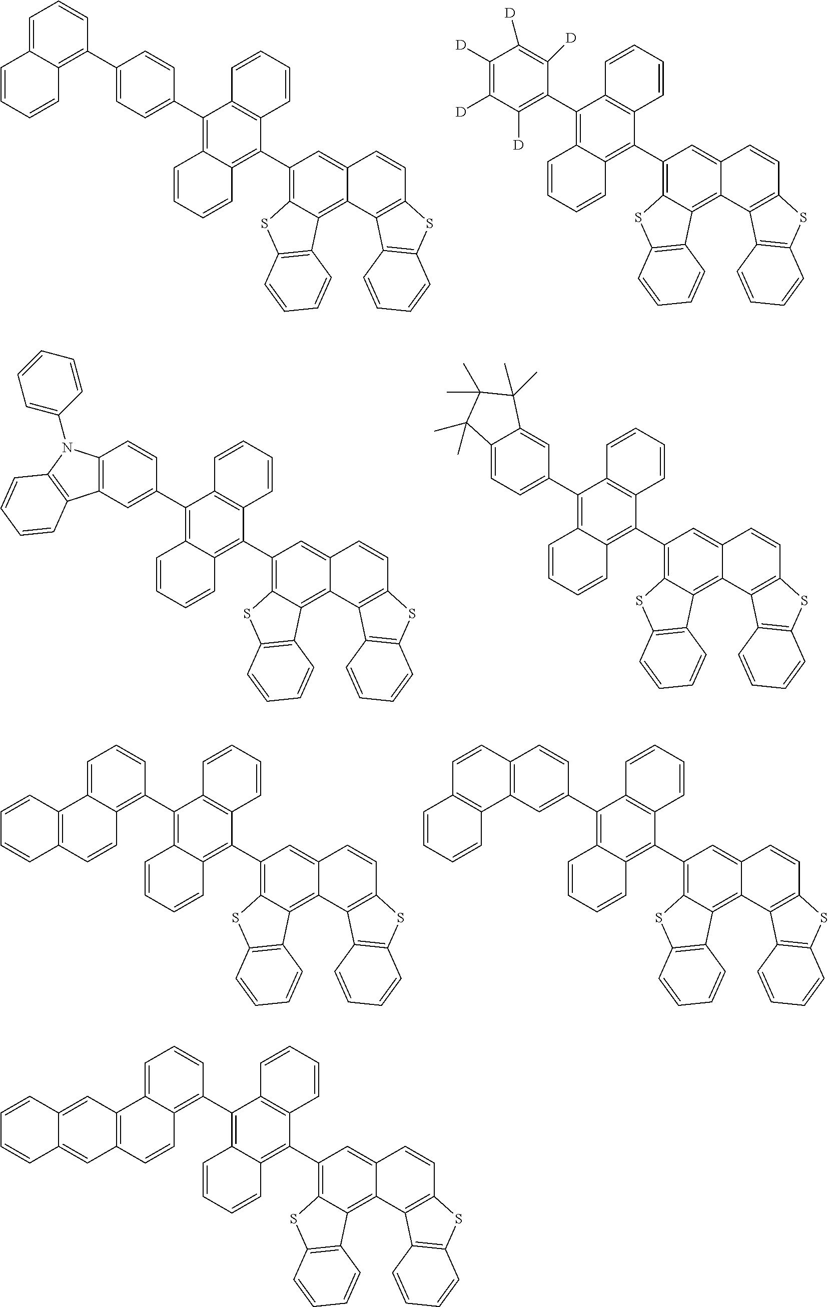

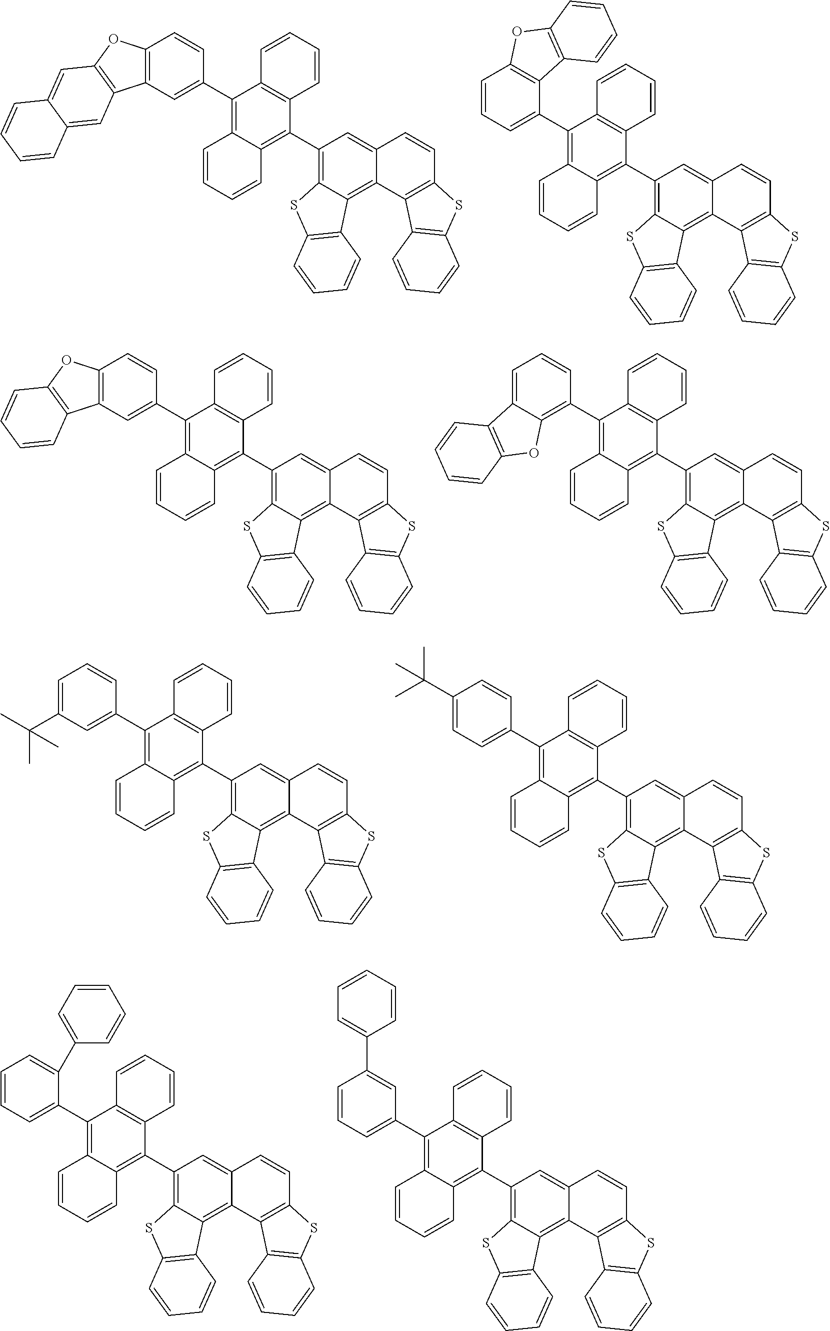

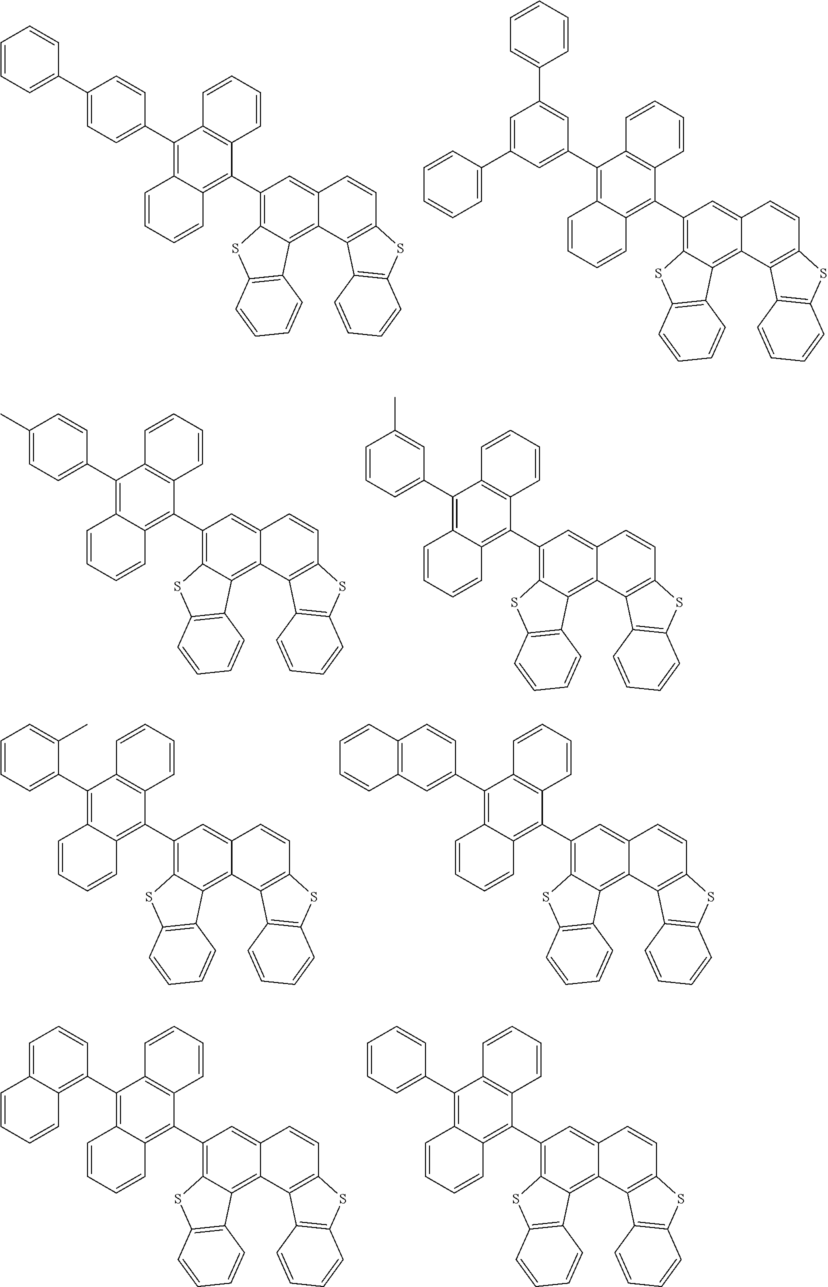

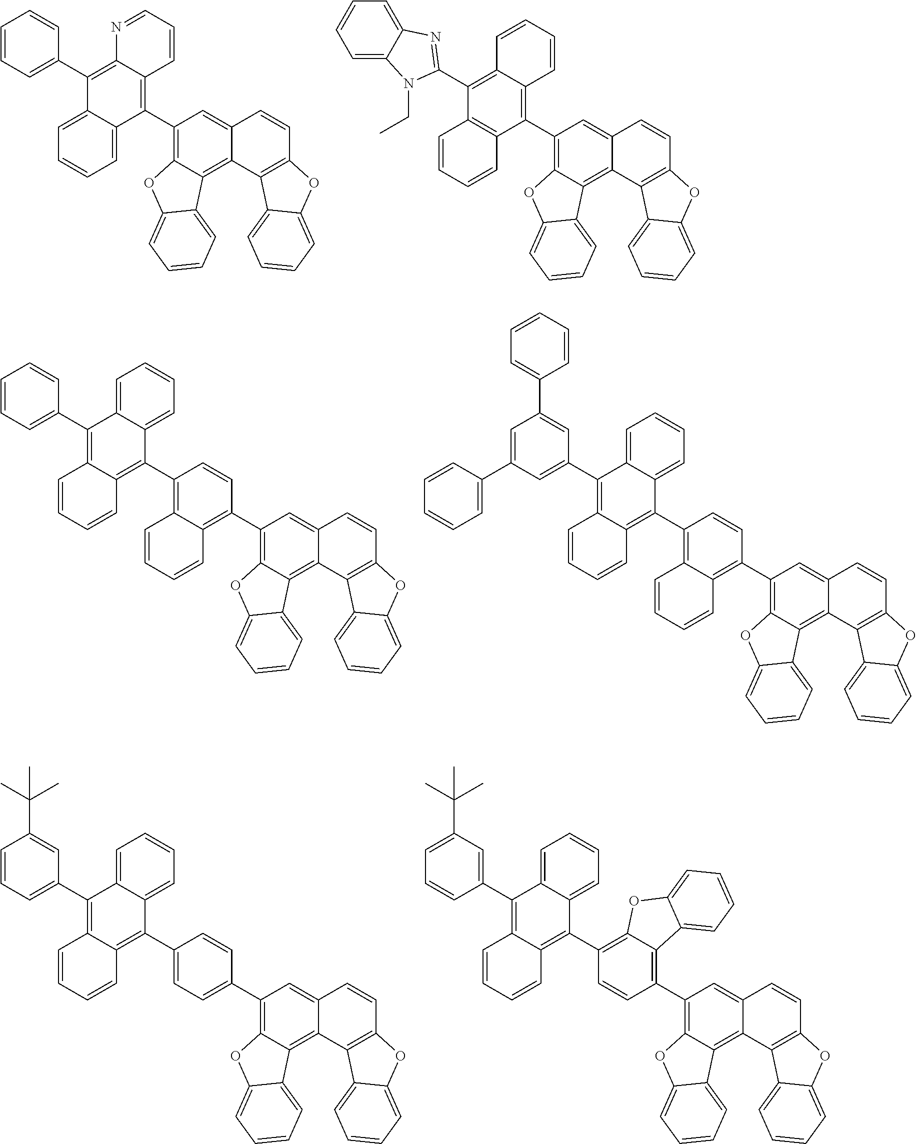

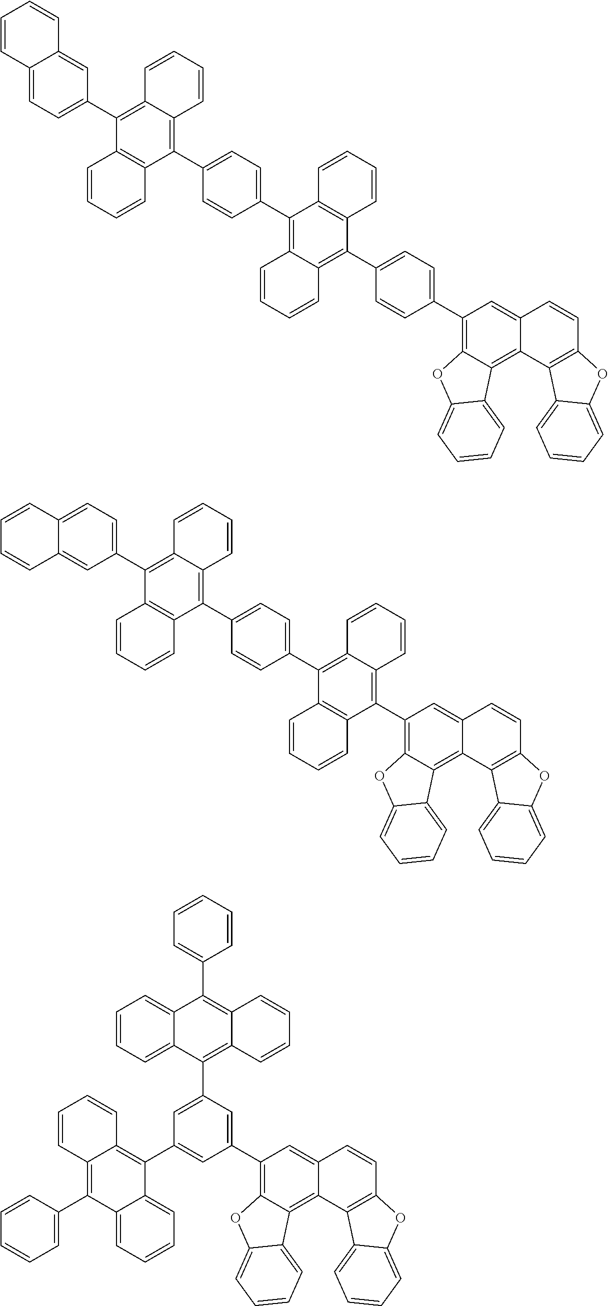 [SMILES: c1ccc2cc(-c3c4ccccc4c(-c4ccc(-c5c6ccccc6c(-c6ccc(-c7cc8ccc9oc%10ccccc%10c9c8c8c7oc7ccccc78)cc6)c6ccccc56)cc4)c4ccccc34)ccc2c1 ; c1ccc2cc(-c3c4ccccc4c(-c4ccc(-c5c6ccccc6c(-c6cc7ccc8oc9ccccc9c8c7c7c6oc6ccccc67)c6ccccc56)cc4)c4ccccc34)ccc2c1 ; c1ccc(-c2c3ccccc3c(-c3cc(-c4c5ccccc5c(-c5ccccc5)c5ccccc45)cc(-c4cc5ccc6oc7ccccc7c6c5c5c4oc4ccccc45)c3)c3ccccc23)cc1]

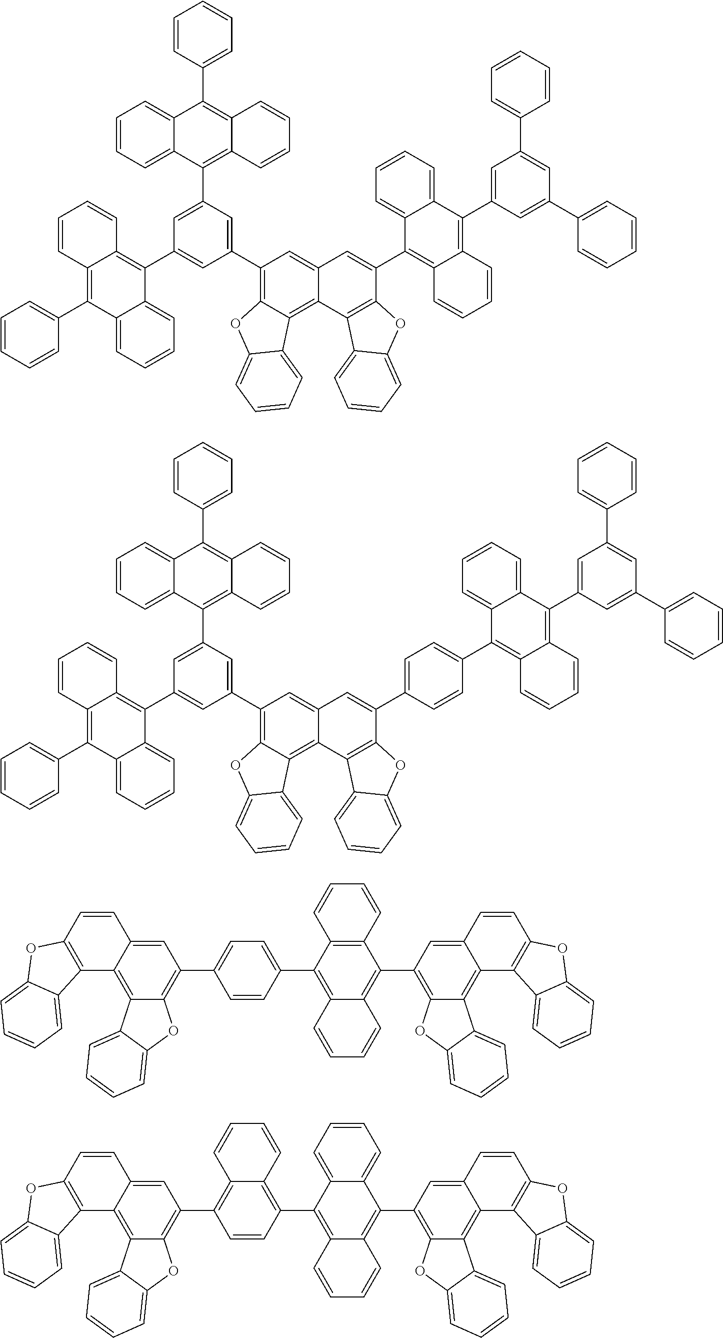

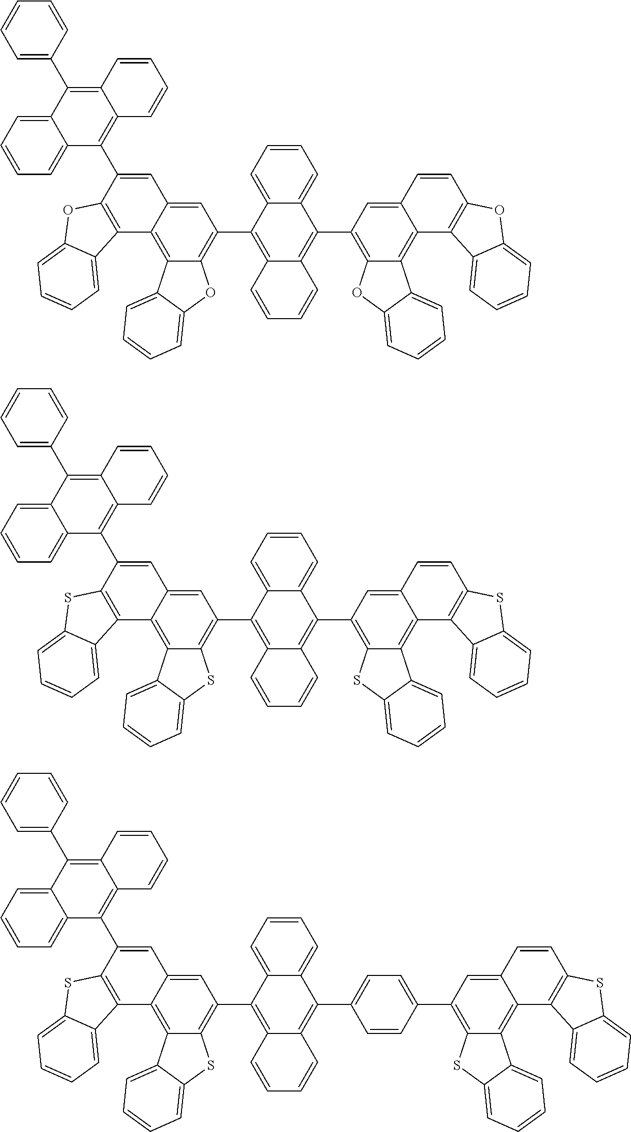 [SMILES: c1ccc(-c2c3ccccc3c(-c3cc4cc(-c5c6ccccc6c(-c6cc7ccc8oc9ccccc9c8c7c7c6oc6ccccc67)c6ccccc56)c5oc6ccccc6c5c4c4c3oc3ccccc34)c3ccccc23)cc1 ; c1ccc(-c2c3ccccc3c(-c3cc4cc(-c5c6ccccc6c(-c6cc7ccc8sc9ccccc9c8c7c7c6sc6ccccc67)c6ccccc56)c5sc6ccccc6c5c4c4c3sc3ccccc34)c3ccccc23)cc1 ; c1ccc(-c2c3ccccc3c(-c3cc4cc(-c5c6ccccc6c(-c6ccc(-c7cc8ccc9sc%10ccccc%10c9c8c8c7sc7ccccc78)cc6)c6ccccc56)c5sc6ccccc6c5c4c4c3sc3ccccc34)c3ccccc23)cc1]

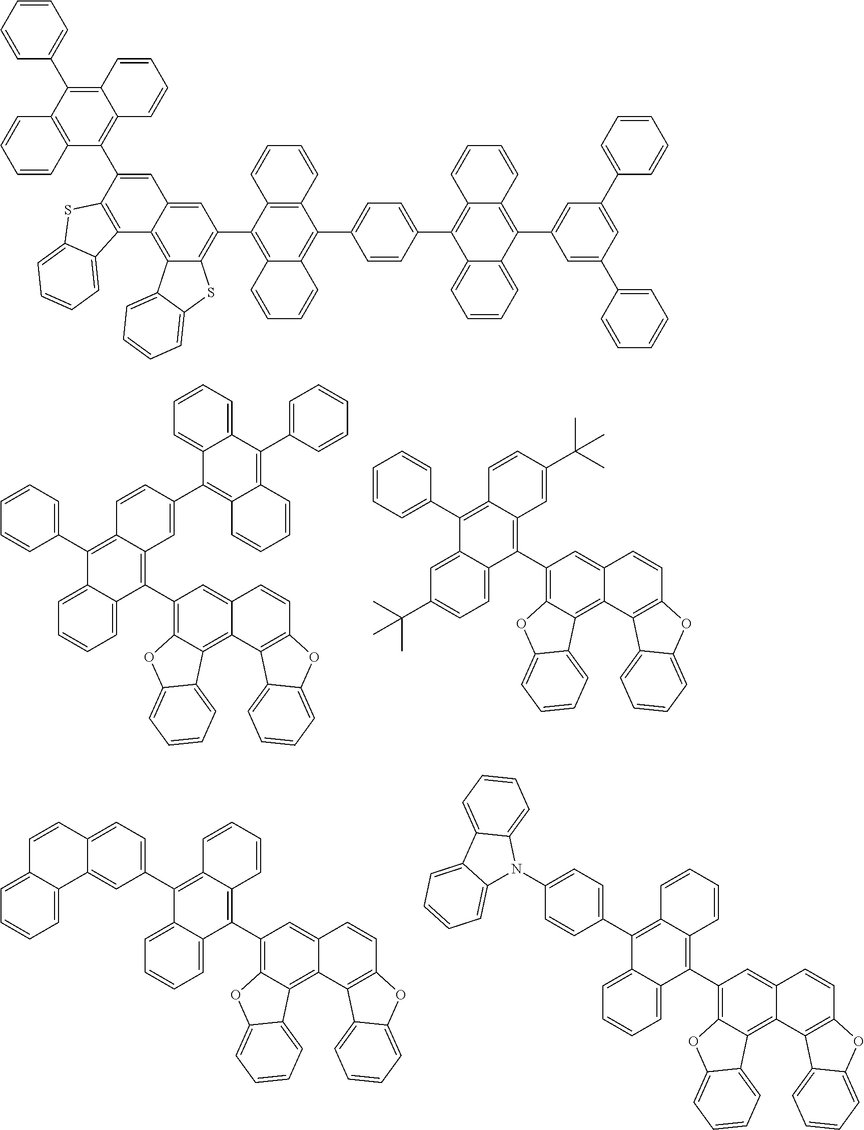

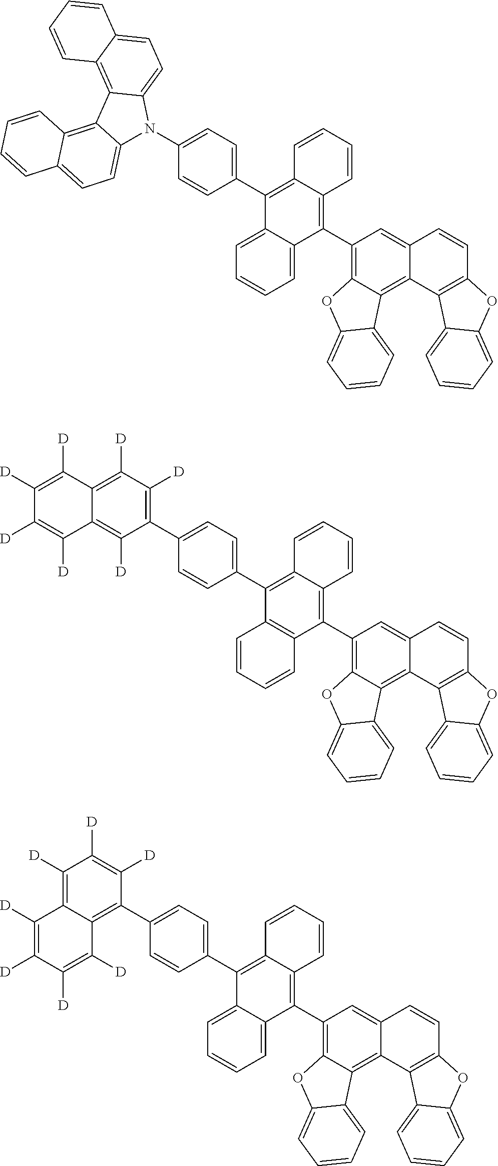 [SMILES: c1ccc2c(c1)ccc1c2c2c3ccccc3ccc2n1-c1ccc(-c2c3ccccc3c(-c3cc4ccc5oc6ccccc6c5c4c4c3oc3ccccc34)c3ccccc23)cc1 ; [2H]c1c([2H])c([2H])c2c([2H])c(-c3ccc(-c4c5ccccc5c(-c5cc6ccc7oc8ccccc8c7c6c6c5oc5ccccc56)c5ccccc45)cc3)c([2H])c([2H])c2c1[2H] ; [2H]c1c([2H])c([2H])c2c(-c3ccc(-c4c5ccccc5c(-c5cc6ccc7oc8ccccc8c7c6c6c5oc5ccccc56)c5ccccc45)cc3)c([2H])c([2H])c([2H])c2c1[2H]]

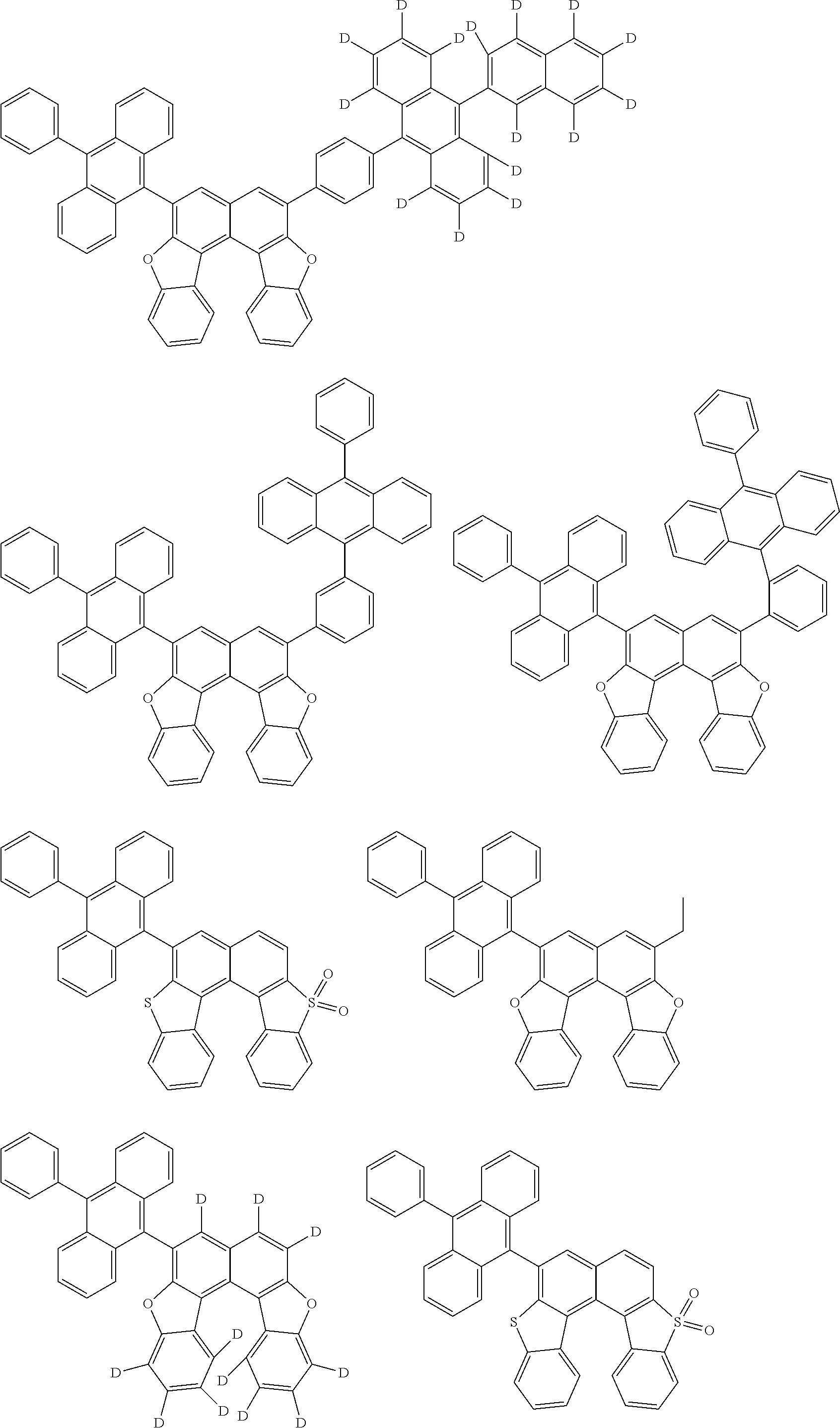

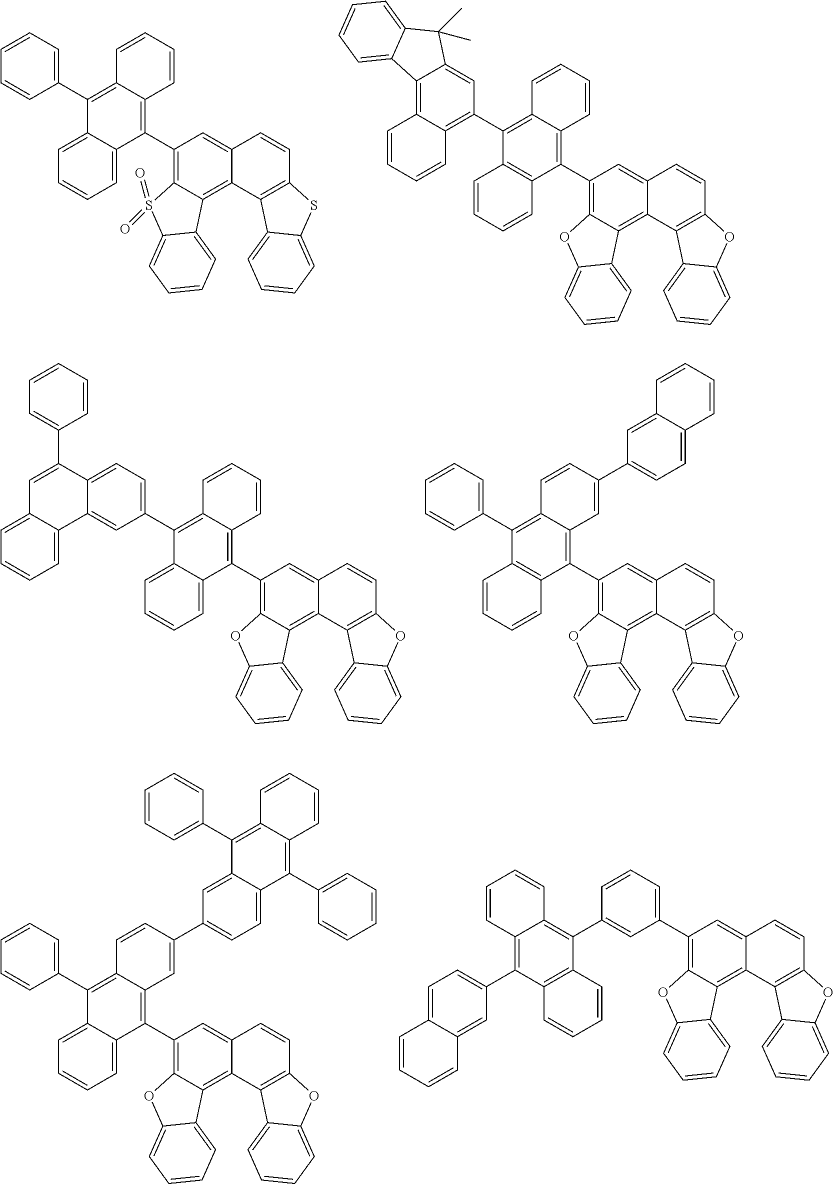

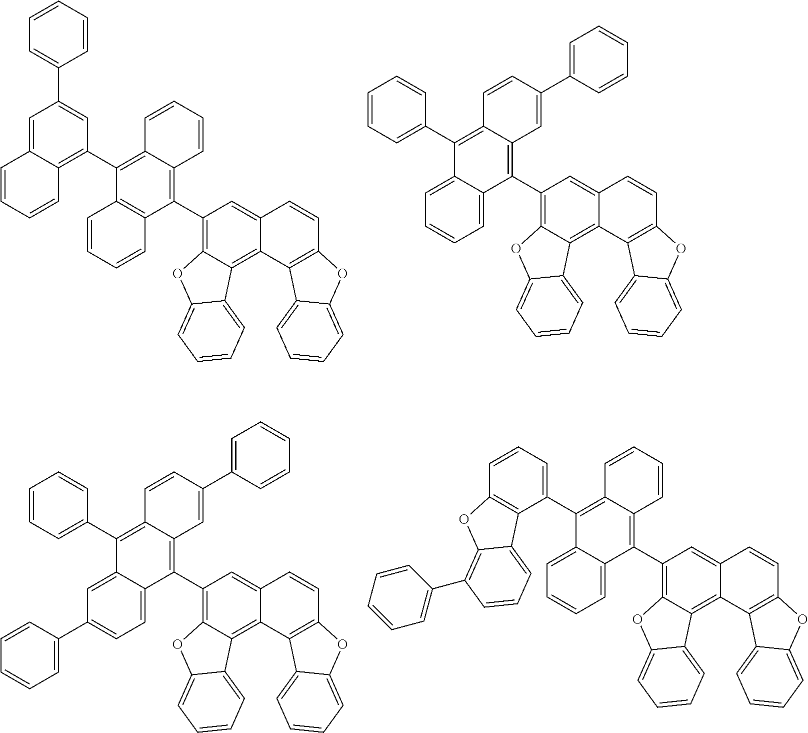

The compounds according to the invention can be prepared by synthesis steps known to the person skilled in the art, such as, for example, bromination, Suzuki coupling, Ullmann coupling, Hartwig-Buchwald coupling, etc.

Examples of suitable synthesis processes for the compounds of formula (1) are detailed in the experimental part below.

The present invention also relates to a process for the synthesis of the compounds of formula (1), which comprises one of the following synthesis routes a1), a2), a3) or a4):

Route a1):

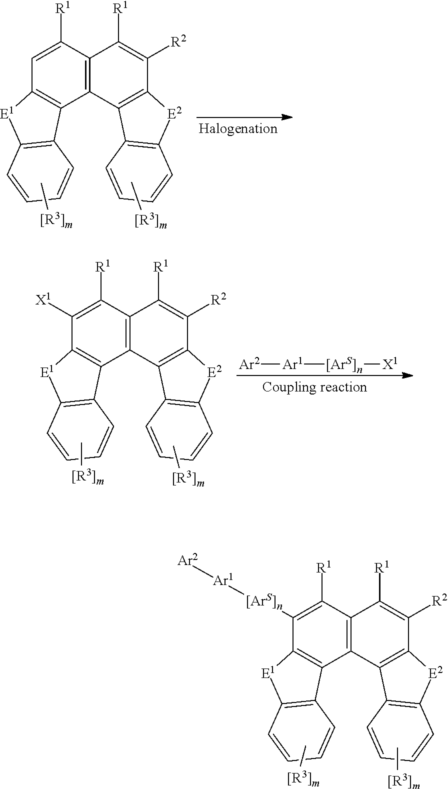

Route a2):

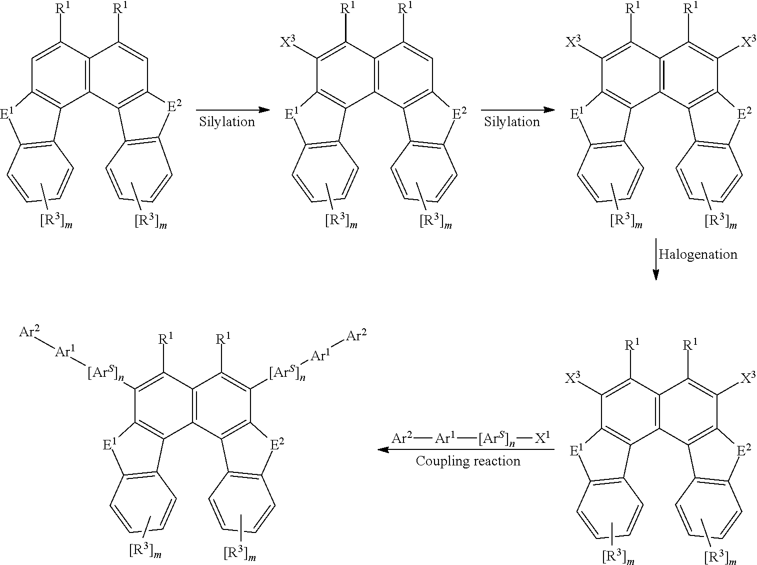

Route a3)

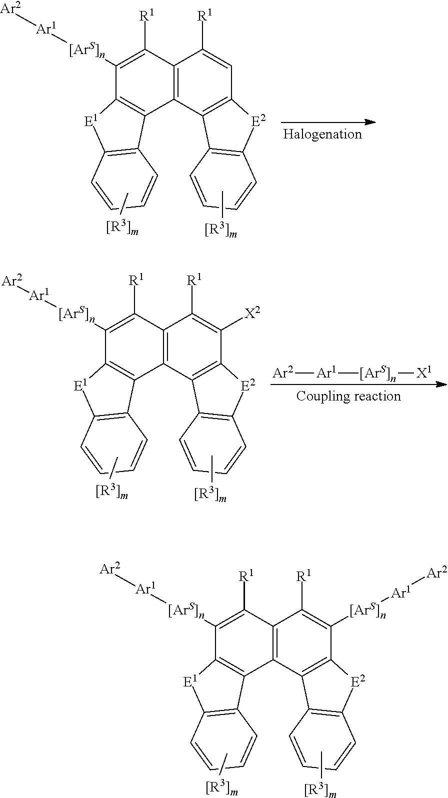

Route a4):

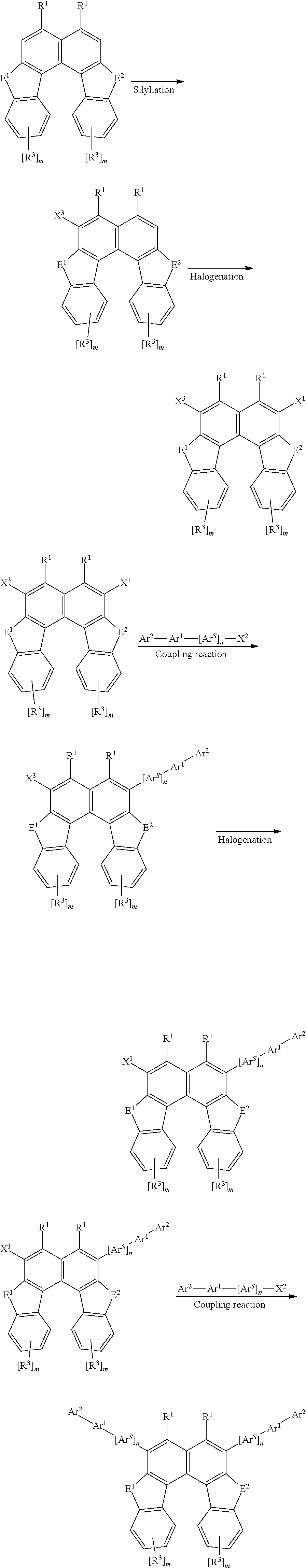

where the symbols R¹, R², R³, Ar¹, Ar², Ar^{S}, E¹, E² and the indices m and n have the same meaning as above, and where:
X¹ is a leaving group selected from halogens, for example I, Br, Cl and F, and triflate;
X² is a leaving group selected from boronic acids and boronic esters, for example boronic acid trimethylene glycol ester, boronic acid ethylene glycol ester, boronic acid pinacol ester, diisopropoxymethylborane, triisoproxymethylborane, boronic acid neo pentyl ester and their derivatives;
X³ is a leaving group selected from silyl groups, for example trimethylsilyl (TMS), triethylsilyl (TES), tert-butyldimethylsilyl (TBDMS), triisopropylsilyl (TIPS), tert-butyldiphenylsilyl (TBDPS), isopropyldimethylsilyl (IPDMS), Diethylisopropylsilyl (DEIPS), triisopropylsilyl (TPS) or Diphenylmethylsilyl (DPMS).

Alternatives to Route a1), Route a2) and Route a3) are Route b1), Route b2) and Route b3) as follows:

Route b1):

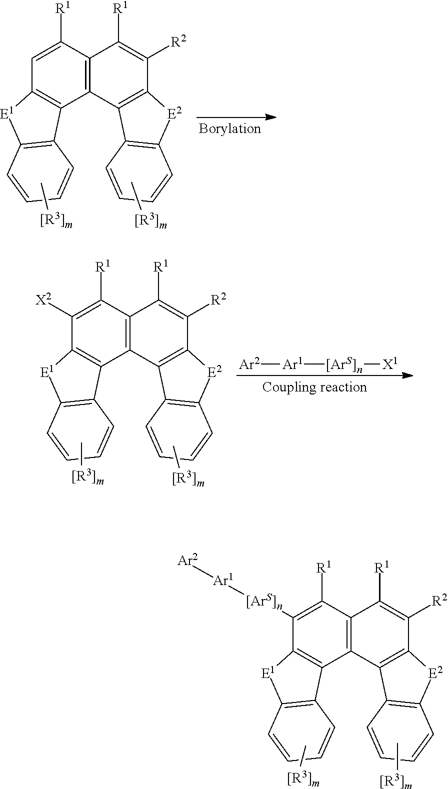

Route b2):

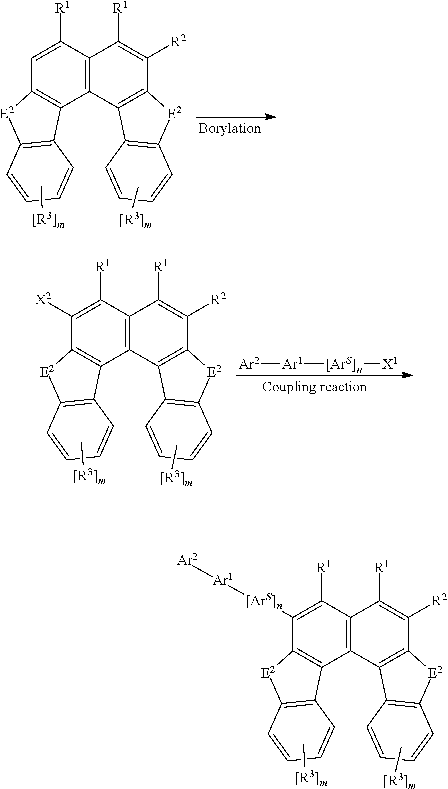

Route b3):

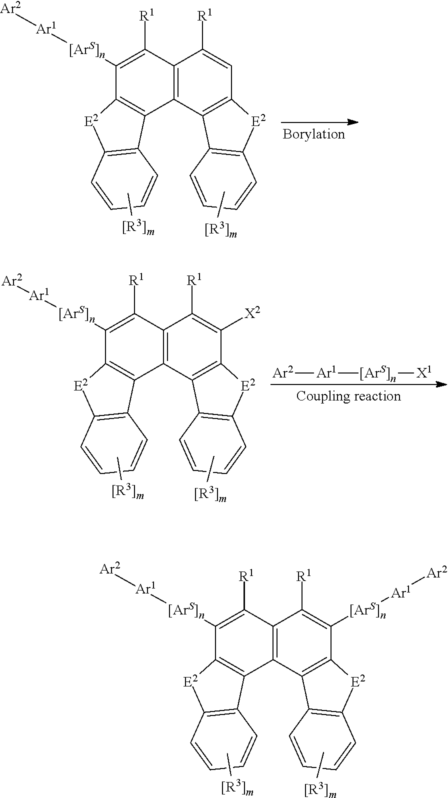

where the symbols and indices in Route b1), Route b2) and Route b3) have the same meaning as above.

The present invention also relates to the intermediates of formulae (Int-1), (Int-2), (Int-3), (Int-4) and (Int-5), which are suitable intermediates for the synthesis of the compounds of formula (1),

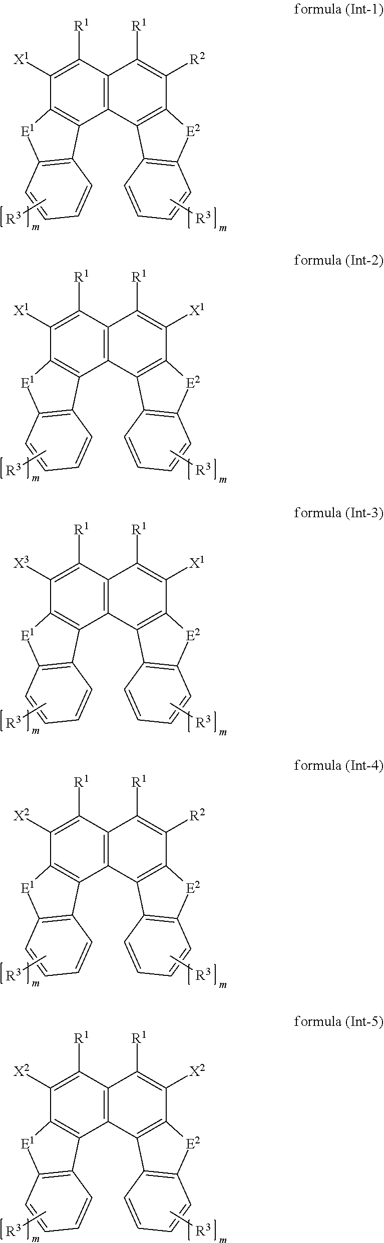

where the symbols R¹, R², R³, E¹, E², X¹, X², X³ and the indices m and n have the same meaning as above.

The above-described compounds, especially compounds substituted by reactive leaving groups, such as bromine, iodine, chlorine, boronic acid or boronic ester, may find use as monomers for production of corresponding oligomers, dendrimers or polymers. Suitable reactive leaving groups are, for example, bromine, iodine, chlorine, boronic acids, boronic esters, amines, alkenyl or alkynyl groups having a terminal C—C double bond or C—C triple bond, oxiranes, oxetanes, groups which enter into a cycloaddition, for example a 1,3-dipolar cycloaddition, for example dienes or azides, carboxylic acid derivatives, alcohols and silanes.

The invention therefore further provides oligomers, polymers or dendrimers containing one or more compounds of formula (1), wherein the bond(s) to the polymer, oligomer or dendrimer may be localized at any desired positions substituted by R, R¹, R² or R³ in the formulae. According to the linkage of the compound, the compound is part of a side chain of the oligomer or polymer or part of the main chain. An oligomer in the context of this invention is understood to mean a compound formed from at least three monomer units. A polymer in the context of the invention is understood to mean a compound formed from at least ten monomer units. The polymers, oligomers or dendrimers of the invention may be conjugated, partly conjugated or nonconjugated. The oligomers or polymers of the invention may be linear, branched or dendritic. In the structures having linear linkage, the units of the above formulae may be joined directly to one another, or they may be joined to one another via a bivalent group, for example via a substituted or unsubstituted alkylene group, via a heteroatom or via a bivalent aromatic or heteroaromatic group. In branched and dendritic structures, it is possible, for example, for three or more units of the above formulae to be joined via a trivalent or higher-valency group, for example via a trivalent or higher-valency aromatic or heteroaromatic group, to give a branched or dendritic oligomer or polymer.

For the repeat units of the above formulae in oligomers, dendrimers and polymers, the same preferences apply as described above for the compounds of the above formulae.

For preparation of the oligomers or polymers, the monomers of the invention are homopolymerized or copolymerized with further monomers. Suitable and preferred comonomers are chosen from fluorenes, spirobifluorenes, paraphenylenes, carbazoles, thiophenes, dihydrophenanthrenes, cis- and trans-indenofluorenes, ketones, phenanthrenes, anthracenes, arylamines or else a plurality of these units. The polymers, oligomers and dendrimers typically contain still further units, for example emitting (fluorescent or phosphorescent) units, for example vinyltriarylamines or phosphorescent metal complexes, and/or charge transport units, especially those based on triarylamines.

The polymers and oligomers of the invention are generally prepared by polymerization of one or more monomer types, of which at least one monomer leads to repeat units of the above formulae in the polymer. Suitable polymerization reactions are known to those skilled in the art and are described in the literature. Particularly suitable and preferred polymerization reactions which lead to formation of C—C or C—N bonds are the Suzuki polymerization, the Yamamoto polymerization, the Stille polymerization and the Hartwig-Buchwald polymerization.

For the processing of the compounds according to the invention from the liquid phase, for example by spin coating or by printing processes, formulations of the compounds according to the invention are necessary. These formulations can be, for example, solutions, dispersions or emulsions. It may be preferred to use mixtures of two or more solvents for this purpose. The solvents are preferably selected from organic and inorganic solvents, more preferably organic solvents. The solvents are very preferably selected from hydrocarbons, alcohols, esters, ethers, ketones and amines. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, in particular 3-phenoxytoluene, (−)-fenchone, 1,2,3,5-tetramethylbenzene, 1,2,4,5-tetramethylbenzene, 1-methylnaphthalene, 1-ethylnaphthalene, decylbenzene, phenyl naphthalene, menthyl isovalerate, para tolyl isobutyrate, cyclohexal hexanoate, ethyl para toluate, ethyl ortho toluate, ethyl meta toluate, decahydronaphthalene, ethyl 2-methoxybenzoate, dibutylaniline, dicyclohexylketone, isosorbide dimethyl ether, decahydronaphthalene, 2-methylbiphenyl, ethyl octanoate, octyl octanoate, diethyl sebacate, 3,3-dimethylbiphenyl, 1,4-dimethylnaphthalene, 2,2′-dimethylbiphenyl, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclo-hexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexyl-benzene, heptylbenzene, octylbenzene, 1,1-bis(3,4-dimethylphenyl)ethane or mixtures of these solvents.

The present invention therefore furthermore relates to a formulation comprising a compound according to the invention and at least one further compound. The further compound may be, for example, a solvent, in particular one of the above-mentioned solvents or a mixture of these solvents. However, the further compound may also be at least one further organic or inorganic compound which is likewise employed in the electronic device, for example an emitting compound, in particular a phosphorescent dopant, and/or a further matrix material. Suitable emitting compounds and further matrix materials are indicated below in connection with the organic electroluminescent device. This further compound may also be polymeric.

The compounds and mixtures according to the invention are suitable for use in an electronic device. An electronic device here is taken to mean a device which comprises at least one layer which comprises at least one organic compound. However, the component here may also comprise inorganic materials or also layers built up entirely from inorganic materials.

The present invention therefore furthermore relates to the use of the compounds or mixtures according to the invention in an electronic device, in particular in an organic electroluminescent device.

The present invention again furthermore relates to an electronic device comprising at least one of the compounds or mixtures according to the invention mentioned above. The preferences stated above for the compound also apply to the electronic devices.

The electronic device is preferably selected from the group consisting of organic electroluminescent devices (OLEDs, PLEDs), organic integrated circuits (O-ICs), organic field-effect transistors (O-FETs), organic thin-film transistors (O-TFTs), organic light-emitting transistors (O-LETs), organic solar cells (O-SCs), organic dye-sensitised solar cells, organic optical detectors, organic photoreceptors, organic field-quench devices (O-FQDs), light-emitting electrochemical cells (LECs), organic laser diodes (O-lasers) and “organic plasmon emitting devices” (D. M. Koller et al., Nature Photonics 2008, 1-4), preferably organic electroluminescent devices (OLEDs, PLEDs), in particular phosphorescent OLEDs.

The organic electroluminescent device comprises a cathode, an anode and at least one emitting layer. Apart from these layers, it may also comprise further layers, for example in each case one or more hole-injection layers, hole-transport layers, hole-blocking layers, electron-transport layers, electron-injection layers, exciton-blocking layers, electron-blocking layers and/or charge-generation layers. It is likewise possible for interlayers, which have, for example, an exciton-blocking function, to be introduced between two emitting layers. However, it should be pointed out that each of these layers does not necessarily have to be present. The organic electroluminescent device here may comprise one emitting layer or a plurality of emitting layers. If a plurality of emission layers are present, these preferably have in total a plurality of emission maxima between 380 nm and 750 nm, resulting overall in white emission, i.e. various emitting compounds which are able to fluoresce or phosphoresce are used in the emitting layers. Particular preference is given to systems having three emitting layers, where the three layers exhibit blue, green and orange or red emission (for the basic structure see, for example, WO 2005/011013). These can be fluorescent or phosphorescent emission layers or hybrid systems, in which fluorescent and phosphorescent emission layers are combined with one another.

The compound according to the invention in accordance with the embodiments indicated above can be employed in various layers, depending on the precise structure and on the substitution. Preference is given to an organic electroluminescent device comprising a compound of the formula (1) or in accordance with the preferred embodiments as fluorescent emitters, emitters showing TADF (Thermally Activated Delayed Fluorescence), matrix materials for fluorescent emitters. Particularly preferred is an organic electroluminescent device comprising a compound of the formula (1) or in accordance with the preferred embodiments as matrix material for fluorescent emitters, more particularly for blue-emitting fluorescent emitters.

The compounds of formula (1) can also be employed in an electron-transport layer and/or in an electron-blocking or exciton-blocking layer and/or in a hole-transport layer, depending on the precise substitution. The preferred embodiments indicated above also apply to the use of the materials in organic electronic devices.

The compound according to the invention is particularly suitable for use as a matrix material for a fluorescent emitting compound.

A matrix material here is taken to mean a material which is present in the emitting layer, preferably as the principal component, and which does not emit light on operation of the device.

The proportion of the emitting compound in the mixture of the emitting layer is between 0.1 and 50.0%, preferably between 0.5 and 20.0%, particularly preferably between 1.0 and 10.0%. Correspondingly, the proportion of the matrix material or matrix materials is between 50.0 and 99.9%, preferably between 80.0 and 99.5%, particularly preferably between 90.0 and 99.0%.

The specifications of the proportions in % are, for the purposes of the present application, taken to mean % by vol. if the compounds are applied from the gas phase and % by weight if the compounds are applied from solution.

If the compound according to the invention is employed as a matrix material for a fluorescent emitting compound in an emitting layer, it may be employed in combination with one or more fluorescent emitting compounds.

Preferred fluorescent emitters are selected from the class of the arylamines. An arylamine in the sense of this invention is taken to mean a compound which contains three substituted or unsubstituted aromatic or heteroaromatic ring systems bonded directly to the nitrogen. At least one of these aromatic or heteroaromatic ring systems is preferably a condensed ring system, particularly preferably having at least 14 aromatic ring atoms. Preferred examples thereof are aromatic anthracenamines, aromatic anthracenediamines, aromatic pyrenamines, aromatic pyrenediamines, aromatic chrysenamines or aromatic chrysenediamines. An aromatic anthracenamine is taken to mean a compound in which one diarylamino group is bonded directly to an anthracene group, preferably in the 9-position. An aromatic anthracene-diamine is taken to mean a compound in which two diarylamino groups are bonded directly to an anthracene group, preferably in the 9,10-position. Aromatic pyrenamines, pyrenediamines, chrysenamines and chrysenediamines are defined analogously thereto, where the diarylamino groups are preferably bonded to the pyrene in the 1-position or in the 1,6-position. Further preferred emitters are indenofluorenamines or indenofluorene-diamines, for example in accordance with WO 2006/108497 or WO 2006/122630, benzoindenofluorenamines or benzoindenofluorenediamines, for example in accordance with WO 2008/006449, and dibenzoindenofluorenamines or dibenzoindenofluorenediamines, for example in accordance with WO 2007/140847, and the indenofluorene derivatives containing condensed aryl groups which are disclosed in WO 2010/012328. Still further preferred emitters are benzanthracene derivatives as disclosed in WO 2015/158409, anthracene derivatives as disclosed in WO 2017/036573, fluorene dimers like in WO 2016/150544 or phenoxazine derivatives as disclosed in WO 2017/028940 and WO 2017/028941. Preference is likewise given to the pyrenarylamines disclosed in WO 2012/048780 and WO 2013/185871. Preference is likewise given to the benzoindenofluorenamines disclosed in WO 2014/037077, the benzofluorenamines disclosed in WO 2014/106522 and the indenofluorenes disclosed in WO 2014/111269 or WO 2017/036574.

Examples of preferred fluorescent emitting compounds, besides the compounds according to the invention, which can be used in combination with the compounds of the invention in an emitting layer or which can be used in another emitting layer of the same device are depicted in the following table:

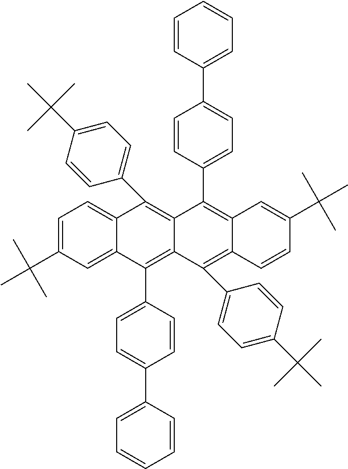 [SMILES: CC(C)(C)c1ccc(-c2c3ccc(C(C)(C)C)cc3c(-c3ccc(-c4ccccc4)cc3)c3c(-c4ccc(C(C)(C)C)cc4)c4ccc(C(C)(C)C)cc4c(-c4ccc(-c5ccccc5)cc4)c23)cc1]

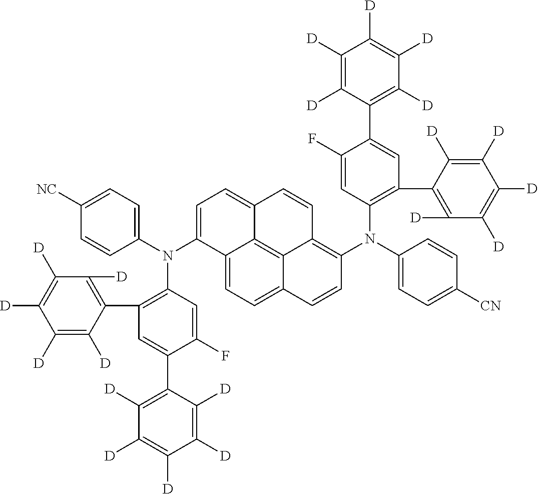 [SMILES: [2H]c1c([2H])c([2H])c(-c2cc(-c3c([2H])c([2H])c([2H])c([2H])c3[2H])c(N(c3ccc(C#N)cc3)c3ccc4ccc5c(N(c6ccc(C#N)cc6)c6cc(F)c(-c7c([2H])c([2H])c([2H])c([2H])c7[2H])cc6-c6c([2H])c([2H])c([2H])c([2H])c6[2H])ccc6ccc3c4c65)cc2F)c([2H])c1[2H]]

| | |
|---|---|
| 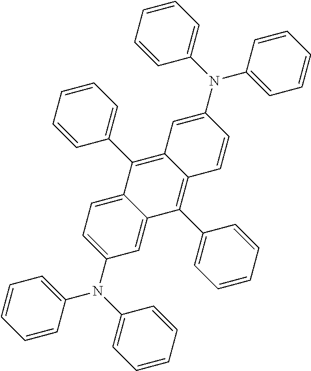 [SMILES: c1ccc(-c2c3ccc(N(c4ccccc4)c4ccccc4)cc3c(-c3ccccc3)c3ccc(N(c4ccccc4)c4ccccc4)cc23)cc1] | 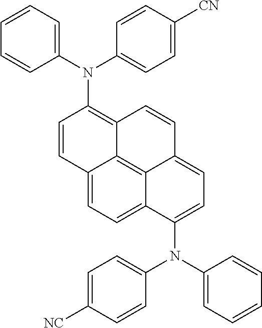 [SMILES: N#Cc1ccc(N(c2ccccc2)c2ccc3ccc4c(N(c5ccccc5)c5ccc(C#N)cc5)ccc5ccc2c3c54)cc1] |
| 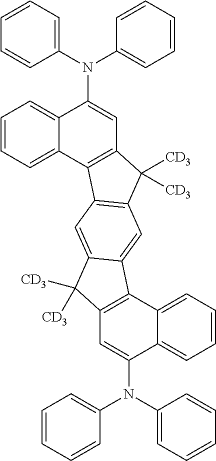 [SMILES: [2H]C([2H])([2H])C1(C([2H])([2H])[2H])c2cc3c(cc2-c2c1cc(N(c1ccccc1)c1ccccc1)c1ccccc21)C(C([2H])([2H])[2H])(C([2H])([2H])[2H])c1cc(N(c2ccccc2)c2ccccc2)c2ccccc2c1-3] | 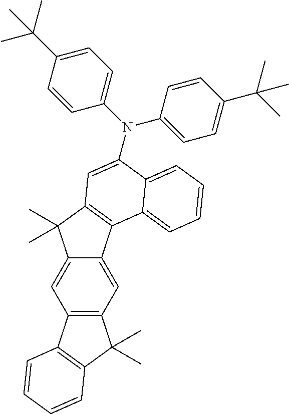 [SMILES: CC(C)(C)c1ccc(N(c2ccc(C(C)(C)C)cc2)c2cc3c(c4ccccc24)-c2cc4c(cc2C3(C)C)-c2ccccc2C4(C)C)cc1] |
| 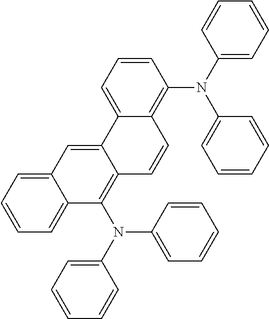 [SMILES: c1ccc(N(c2ccccc2)c2cccc3c2ccc2c(N(c4ccccc4)c4ccccc4)c4ccccc4cc23)cc1] | 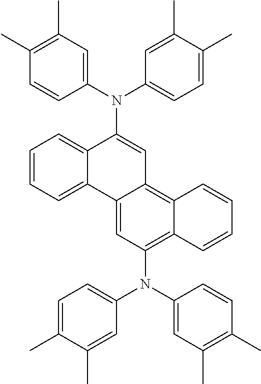 |
| 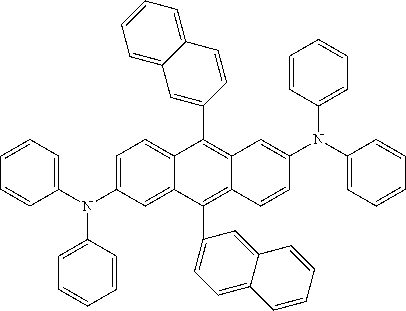 [SMILES: c1ccc(N(c2ccccc2)c2ccc3c(-c4ccc5ccccc5c4)c4cc(N(c5ccccc5)c5ccccc5)ccc4c(-c4ccc5ccccc5c4)c3c2)cc1] | 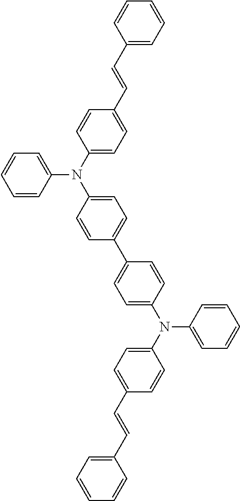 [SMILES: C(=C\c1ccc(N(c2ccccc2)c2ccc(-c3ccc(N(c4ccccc4)c4ccc(/C=C/c5ccccc5)cc4)cc3)cc2)cc1)/c1ccccc1] |
| 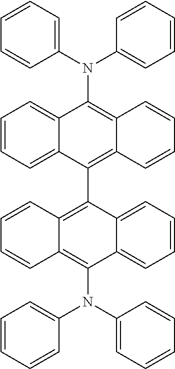 [SMILES: c1ccc(N(c2ccccc2)c2c3ccccc3c(-c3c4ccccc4c(N(c4ccccc4)c4ccccc4)c4ccccc34)c3ccccc23)cc1] | 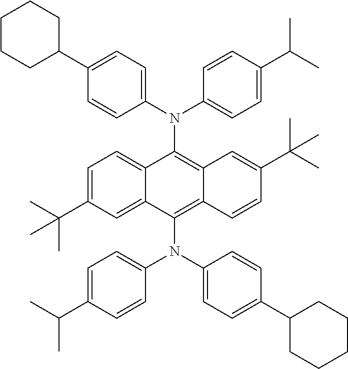 [SMILES: CC(C)c1ccc(N(c2ccc(C3CCCCC3)cc2)c2c3ccc(C(C)(C)C)cc3c(N(c3ccc(C(C)C)cc3)c3ccc(C4CCCCC4)cc3)c3ccc(C(C)(C)C)cc23)cc1] |

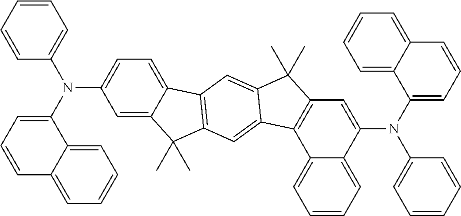 [SMILES: CC1(C)c2cc(N(c3ccccc3)c3cccc4ccccc34)ccc2-c2cc3c(cc21)-c1c(cc(N(c2ccccc2)c2cccc4ccccc24)c2ccccc12)C3(C)C]

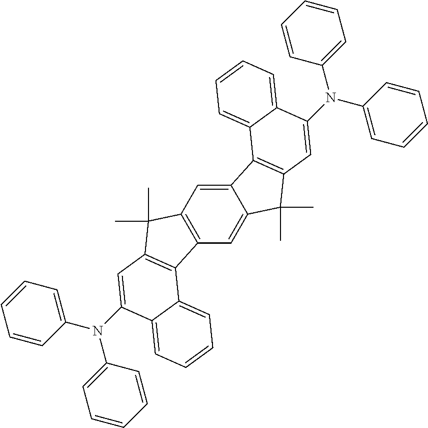 [SMILES: CC1(C)c2cc3c(cc2-c2c1cc(N(c1ccccc1)c1ccccc1)c1ccccc21)C(C)(C)c1cc(N(c2ccccc2)c2ccccc2)c2ccccc2c1-3]

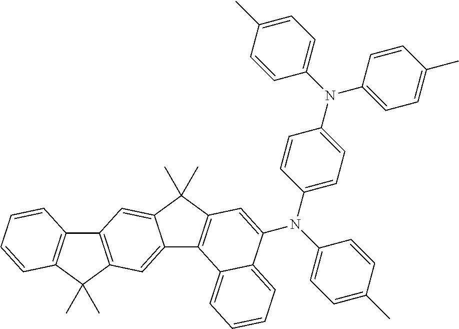 [SMILES: Cc1ccc(N(c2ccc(C)cc2)c2ccc(N(c3ccc(C)cc3)c3cc4c(c5ccccc35)-c3cc5c(cc3C4(C)C)-c3ccccc3C5(C)C)cc2)cc1]

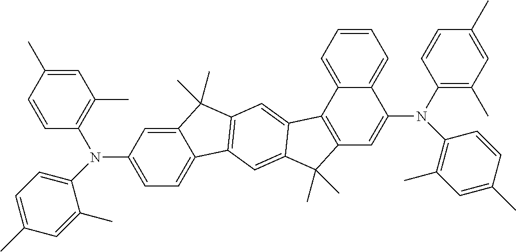

| | |
|---|---|
| 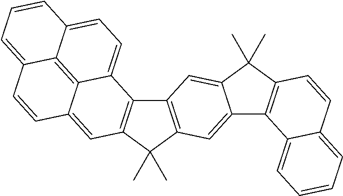 [SMILES: CC1(C)c2cc3c(cc2-c2c1ccc1ccccc21)C(C)(C)c1cc2ccc4cccc5ccc(c1-3)c2c45] | 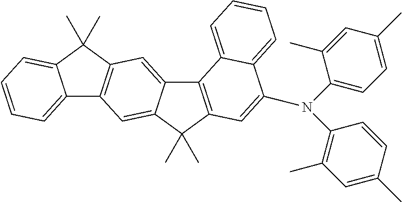 [SMILES: Cc1ccc(N(c2ccc(C)cc2C)c2cc3c(c4ccccc24)-c2cc4c(cc2C3(C)C)-c2ccccc2C4(C)C)c(C)c1] |

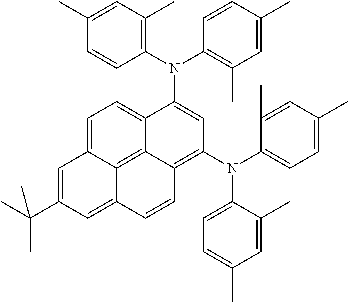 [SMILES: Cc1ccc(N(c2ccc(C)cc2C)c2cc(N(c3ccc(C)cc3C)c3ccc(C)cc3C)c3ccc4cc(C(C)(C)C)cc5ccc2c3c54)c(C)c1]

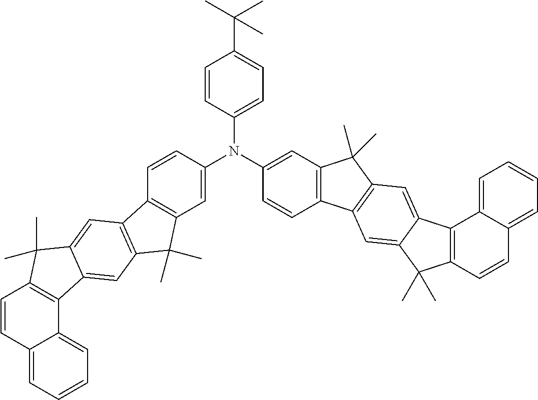 [SMILES: CC(C)(C)c1ccc(N(c2ccc3c(c2)C(C)(C)c2cc4c(cc2-3)C(C)(C)c2ccc3ccccc3c2-4)c2ccc3c(c2)C(C)(C)c2cc4c(cc2-3)C(C)(C)c2ccc3ccccc3c2-4)cc1]

| | |
|---|---|
| 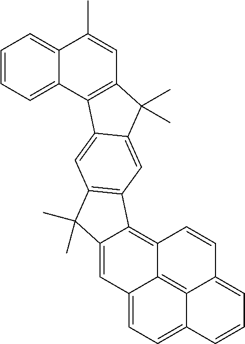 [SMILES: Cc1cc2c(c3ccccc13)-c1cc3c(cc1C2(C)C)-c1c(cc2ccc4cccc5ccc1c2c45)C3(C)C] | 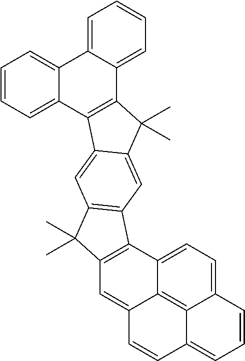 [SMILES: CC1(C)c2cc3c(cc2-c2c1cc1ccc4cccc5ccc2c1c45)C(C)(C)c1c-3c2ccccc2c2ccccc12] |

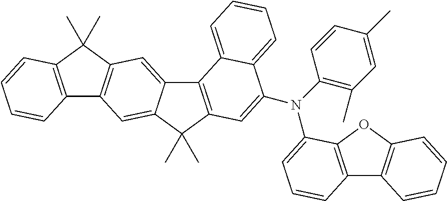 [SMILES: Cc1ccc(N(c2cc3c(c4ccccc24)-c2cc4c(cc2C3(C)C)-c2ccccc2C4(C)C)c2cccc3c2oc2ccccc23)c(C)c1]

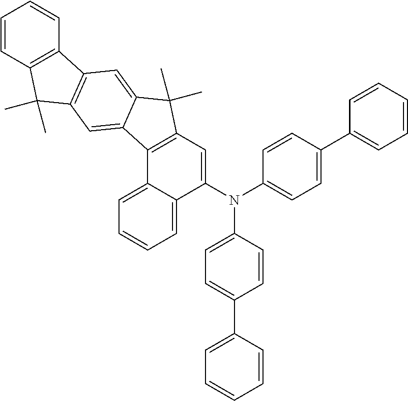 [SMILES: CC1(C)c2ccccc2-c2cc3c(cc21)-c1c(cc(N(c2ccc(-c4ccccc4)cc2)c2ccc(-c4ccccc4)cc2)c2ccccc12)C3(C)C]

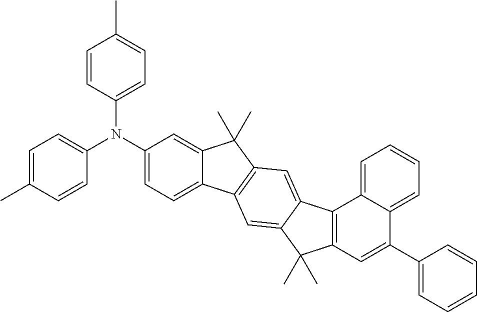 [SMILES: Cc1ccc(N(c2ccc(C)cc2)c2ccc3c(c2)C(C)(C)c2cc4c(cc2-3)C(C)(C)c2cc(-c3ccccc3)c3ccccc3c2-4)cc1]

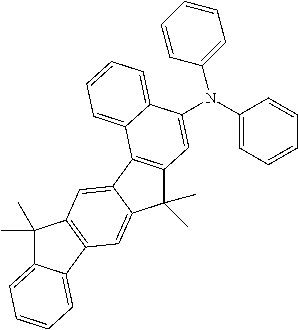 [SMILES: CC1(C)c2ccccc2-c2cc3c(cc21)-c1c(cc(N(c2ccccc2)c2ccccc2)c2ccccc12)C3(C)C]

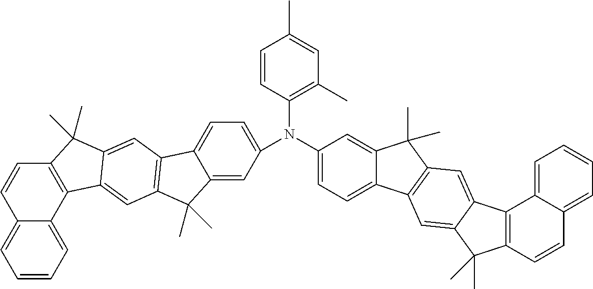 [SMILES: Cc1ccc(N(c2ccc3c(c2)C(C)(C)c2cc4c(cc2-3)C(C)(C)c2ccc3ccccc3c2-4)c2ccc3c(c2)C(C)(C)c2cc4c(cc2-3)C(C)(C)c2ccc3ccccc3c2-4)c(C)c1]

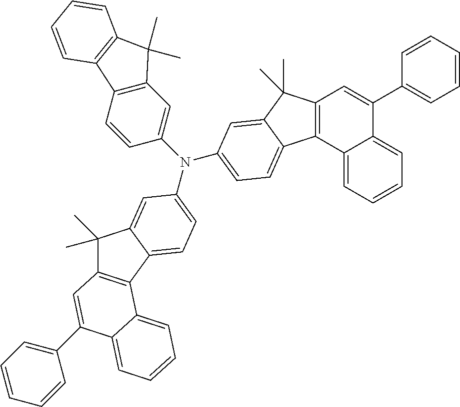 [SMILES: CC1(C)c2ccccc2-c2ccc(N(c3ccc4c(c3)C(C)(C)c3cc(-c5ccccc5)c5ccccc5c3-4)c3ccc4c(c3)C(C)(C)c3cc(-c5ccccc5)c5ccccc5c3-4)cc21]

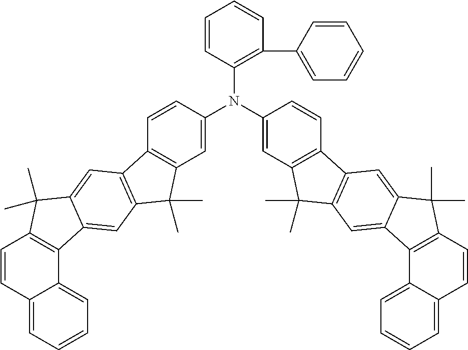 [SMILES: CC1(C)c2cc(N(c3ccc4c(c3)C(C)(C)c3cc5c(cc3-4)C(C)(C)c3ccc4ccccc4c3-5)c3ccccc3-c3ccccc3)ccc2-c2cc3c(cc21)-c1c(ccc2ccccc12)C3(C)C]

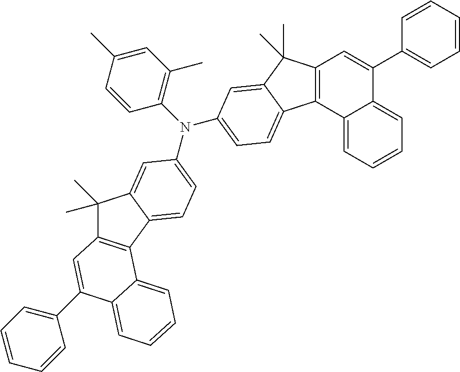 [SMILES: Cc1ccc(N(c2ccc3c(c2)C(C)(C)c2cc(-c4ccccc4)c4ccccc4c2-3)c2ccc3c(c2)C(C)(C)c2cc(-c4ccccc4)c4ccccc4c2-3)c(C)c1]

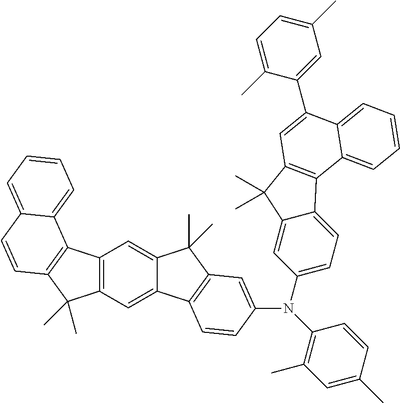 [SMILES: Cc1ccc(N(c2ccc3c(c2)C(C)(C)c2cc4c(cc2-3)C(C)(C)c2ccc3ccccc3c2-4)c2ccc3c(c2)C(C)(C)c2cc(-c4cc(C)ccc4C)c4ccccc4c2-3)c(C)c1]

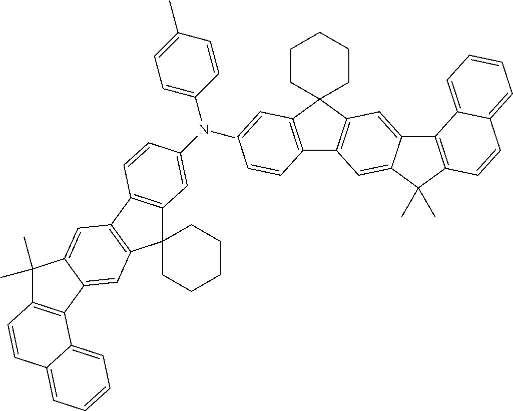

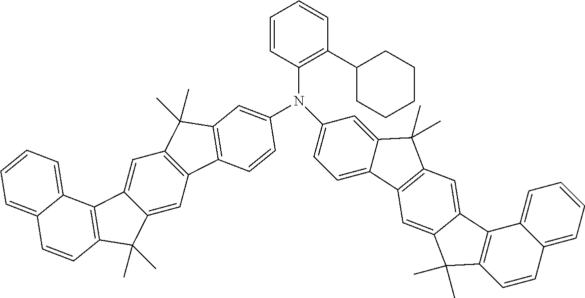 [SMILES: CC1(C)c2cc(N(c3ccc4c(c3)C(C)(C)c3cc5c(cc3-4)C(C)(C)c3ccc4ccccc4c3-5)c3ccccc3C3CCCCC3)ccc2-c2cc3c(cc21)-c1c(ccc2ccccc12)C3(C)C]

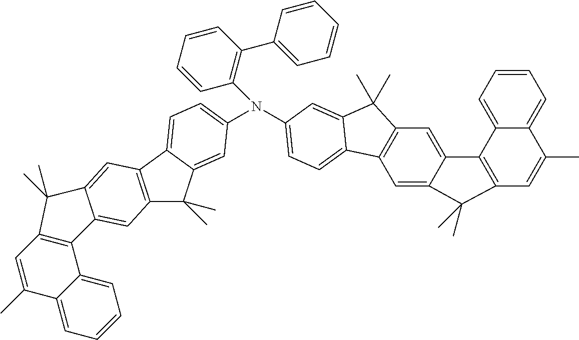

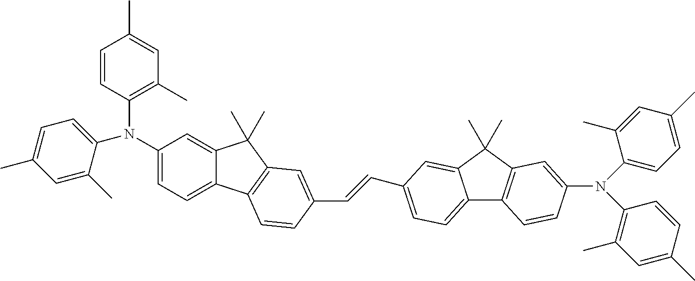

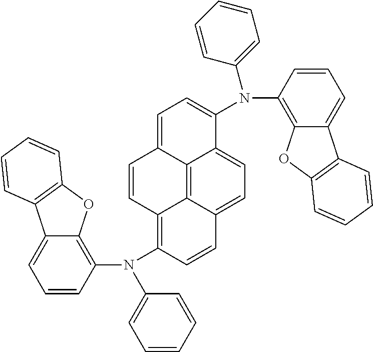 [SMILES: c1ccc(N(c2ccc3ccc4c(N(c5ccccc5)c5cccc6c5oc5ccccc56)ccc5ccc2c3c54)c2cccc3c2oc2ccccc23)cc1]

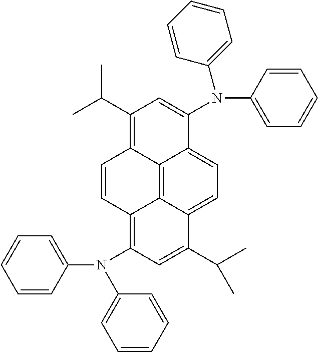 [SMILES: CC(C)c1cc(N(c2ccccc2)c2ccccc2)c2ccc3c(C(C)C)cc(N(c4ccccc4)c4ccccc4)c4ccc1c2c34]

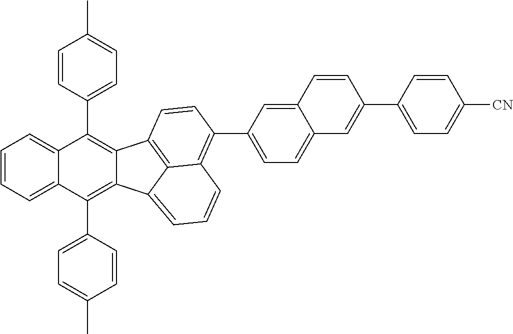 [SMILES: Cc1ccc(-c2c3c(c(-c4ccc(C)cc4)c4ccccc24)-c2ccc(-c4ccc5cc(-c6ccc(C#N)cc6)ccc5c4)c4cccc-3c24)cc1]

| | |
|---|---|
| 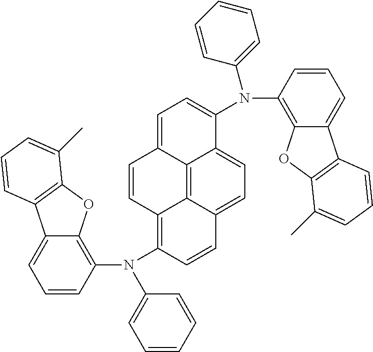 [SMILES: Cc1cccc2c1oc1c(N(c3ccccc3)c3ccc4ccc5c(N(c6ccccc6)c6cccc7c6oc6c(C)cccc67)ccc6ccc3c4c65)cccc12] | 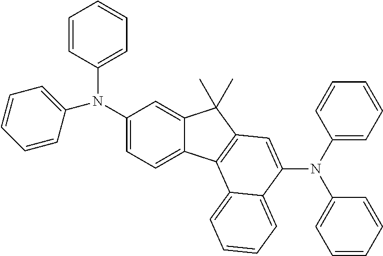 [SMILES: CC1(C)c2cc(N(c3ccccc3)c3ccccc3)ccc2-c2c1cc(N(c1ccccc1)c1ccccc1)c1ccccc21] |

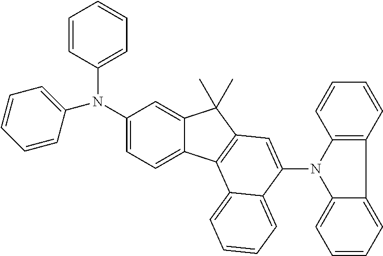 [SMILES: CC1(C)c2cc(N(c3ccccc3)c3ccccc3)ccc2-c2c1cc(-n1c3ccccc3c3ccccc31)c1ccccc21]

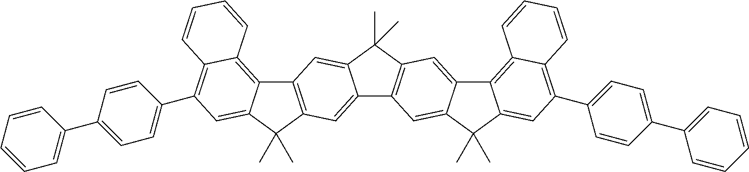 [SMILES: CC1(C)c2cc3c(cc2-c2cc4c(cc21)-c1c(cc(-c2ccc(-c5ccccc5)cc2)c2ccccc12)C4(C)C)C(C)(C)c1cc(-c2ccc(-c4ccccc4)cc2)c2ccccc2c1-3]

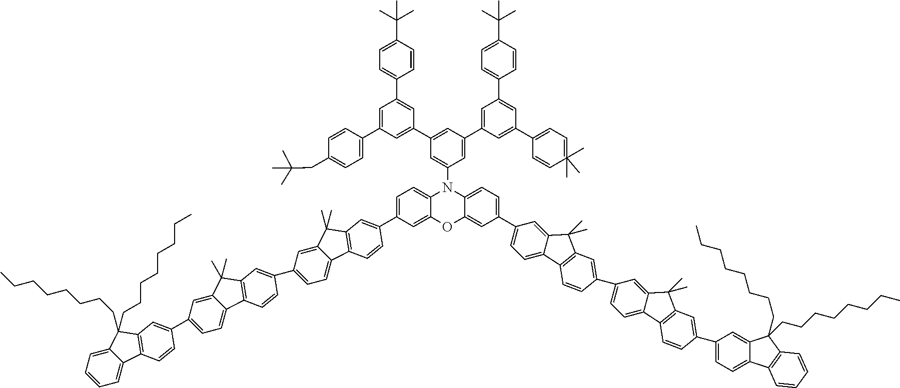

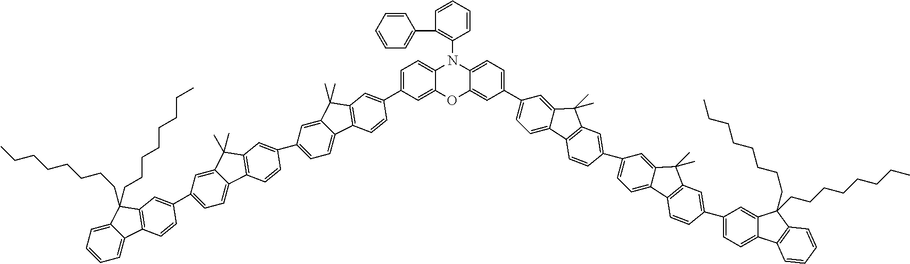 [SMILES: CCCCCCCCC1(CCCCCCCC)c2ccccc2-c2ccc(-c3ccc4c(c3)C(C)(C)c3cc(-c5ccc6c(c5)C(C)(C)c5cc(-c7ccc8c(c7)Oc7cc(-c9ccc%10c(c9)C(C)(C)c9cc(-c%11ccc%12c(c%11)C(C)(C)c%11cc(-c%13ccc%14c(c%13)C(CCCCCCCC)(CCCCCCCC)c%13ccccc%13-%14)ccc%11-%12)ccc9-%10)ccc7N8c7ccccc7-c7ccccc7)ccc5-6)ccc3-4)cc21]

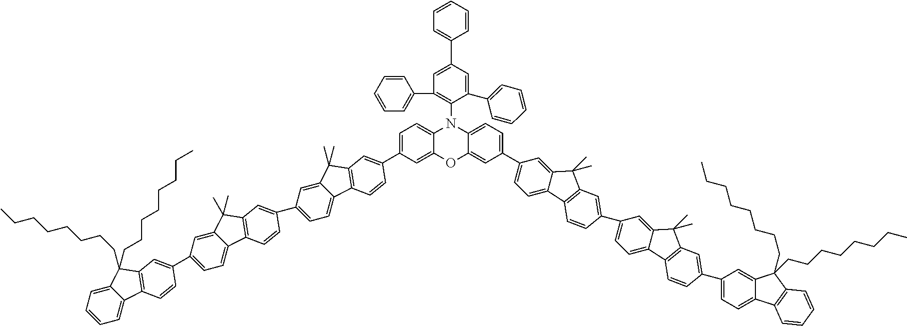 [SMILES: CCCCCCCCC1(CCCCCCCC)c2ccccc2-c2ccc(-c3ccc4c(c3)C(C)(C)c3cc(-c5ccc6c(c5)C(C)(C)c5cc(-c7ccc8c(c7)Oc7cc(-c9ccc%10c(c9)C(C)(C)c9cc(-c%11ccc%12c(c%11)C(C)(C)c%11cc(-c%13ccc%14c(c%13)C(CCCCCCCC)(CCCCCCCC)c%13ccccc%13-%14)ccc%11-%12)ccc9-%10)ccc7N8c7c(-c8ccccc8)cc(-c8ccccc8)cc7-c7ccccc7)ccc5-6)ccc3-4)cc21]

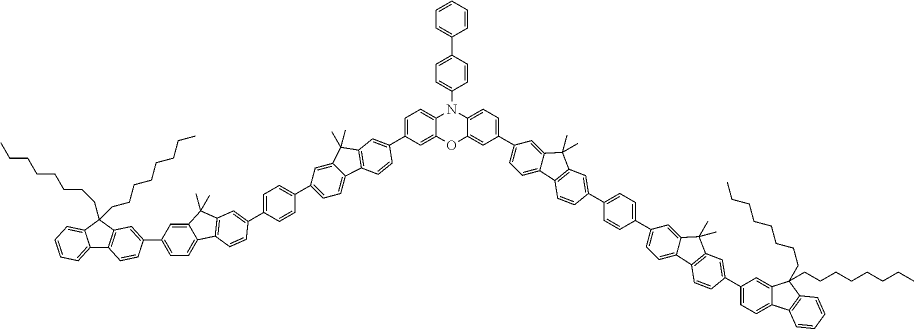 [SMILES: CCCCCCCCC1(CCCCCCCC)c2ccccc2-c2ccc(-c3ccc4c(c3)C(C)(C)c3cc(-c5ccc(-c6ccc7c(c6)C(C)(C)c6cc(-c8ccc9c(c8)Oc8cc(-c%10ccc%11c(c%10)C(C)(C)c%10cc(-c%12ccc(-c%13ccc%14c(c%13)C(C)(C)c%13cc(-c%15ccc%16c(c%15)C(CCCCCCCC)(CCCCCCCC)c%15ccccc%15-%16)ccc%13-%14)cc%12)ccc%10-%11)ccc8N9c8ccc(-c9ccccc9)cc8)ccc6-7)cc5)ccc3-4)cc21]

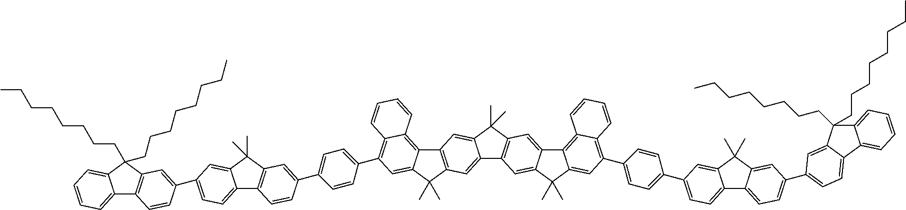 [SMILES: CCCCCCCCC1(CCCCCCCC)c2ccccc2-c2ccc(-c3ccc4c(c3)C(C)(C)c3cc(-c5ccc(-c6cc7c(c8ccccc68)-c6cc8c(cc6C7(C)C)-c6cc7c(cc6C8(C)C)-c6c(cc(-c8ccc(-c9ccc%10c(c9)C(C)(C)c9cc(-c%11ccc%12c(c%11)C(CCCCCCCC)(CCCCCCCC)c%11ccccc%11-%12)ccc9-%10)cc8)c8ccccc68)C7(C)C)cc5)ccc3-4)cc21]

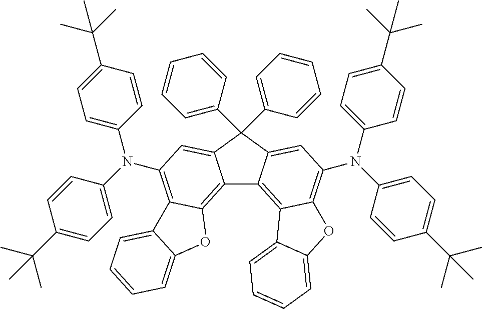 [SMILES: CC(C)(C)c1ccc(N(c2ccc(C(C)(C)C)cc2)c2cc3c(c4c2oc2ccccc24)-c2c(cc(N(c4ccc(C(C)(C)C)cc4)c4ccc(C(C)(C)C)cc4)c4c2oc2ccccc24)C3(c2ccccc2)c2ccccc2)cc1]

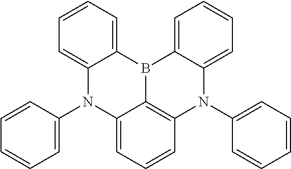 [SMILES: c1ccc(N2c3ccccc3B3c4ccccc4N(c4ccccc4)c4cccc2c43)cc1]

The electronic device concerned may comprise a single emitting layer comprising the compound according to the invention or it may comprise two or more emitting layers. The further emitting layers here may comprise one or more compounds according to the invention or alternatively other compounds.

If the compound according to the invention is employed as a matrix material for a fluorescent emitting compound in an emitting layer, it is may be employed in combination with one or more further matrix materials.

Preferred matrix materials for use in combination with the compound of formula (1) or its preferred embodiments are selected from the classes of the oligoarylenes (for example 2,2′,7,7′-tetraphenylspirobifluorene in accordance with EP 676461 or dinaphthylanthracene), in particular the oligoarylenes containing condensed aromatic groups, the oligoarylenevinylenes (for example DPVBi or spiro-DPVBi in accordance with EP 676461), the polypodal metal complexes (for example in accordance with WO 2004/081017), the hole-conducting compounds (for example in accordance with WO 2004/058911), the electron-conducting compounds, in particular ketones, phosphine oxides, sulfoxides, etc. (for example in accordance with WO 2005/084081 and WO 2005/084082), the atropisomers (for example in accordance with WO 2006/048268), the boronic acid derivatives (for example in accordance with WO 2006/117052) or the benzanthracenes (for example in accordance with WO 2008/145239). Particularly preferred matrix materials are selected from the classes of the oligoarylenes, comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes, comprising anthracene, benzanthracene, benzophenanthrene and/or pyrene or atropisomers of these compounds. An oligoarylene in the sense of this invention is intended to be taken to mean a compound in which at least three aryl or arylene groups are bonded to one another.

Particularly preferred matrix materials for use in combination with the compounds of the formula (1) in the emitting layer are depicted in the following table.

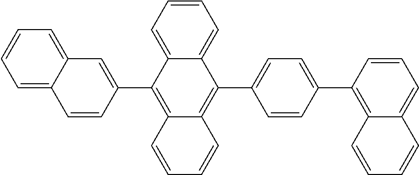 [SMILES: c1ccc2cc(-c3c4ccccc4c(-c4ccc(-c5cccc6ccccc56)cc4)c4ccccc34)ccc2c1]

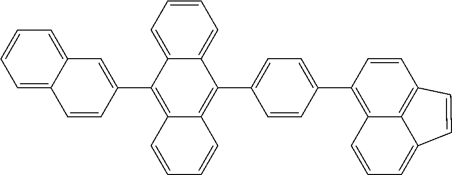 [SMILES: C1=Cc2ccc(-c3ccc(-c4c5ccccc5c(-c5ccc6ccccc6c5)c5ccccc45)cc3)c3cccc1c23]

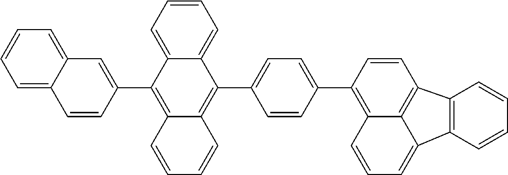 [SMILES: c1ccc2c(c1)-c1cccc3c(-c4ccc(-c5c6ccccc6c(-c6ccc7ccccc7c6)c6ccccc56)cc4)ccc-2c13]

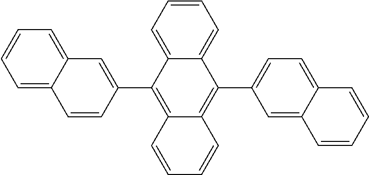 [SMILES: c1ccc2cc(-c3c4ccccc4c(-c4ccc5ccccc5c4)c4ccccc34)ccc2c1]

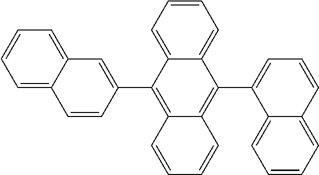 [SMILES: c1ccc2cc(-c3c4ccccc4c(-c4cccc5ccccc45)c4ccccc34)ccc2c1]

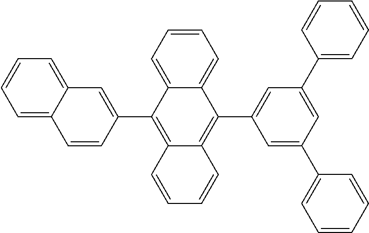 [SMILES: c1ccc(-c2cc(-c3ccccc3)cc(-c3c4ccccc4c(-c4ccc5ccccc5c4)c4ccccc34)c2)cc1]

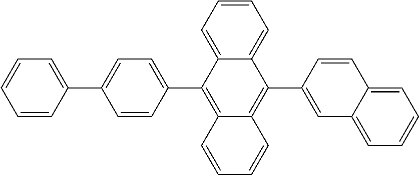 [SMILES: c1ccc(-c2ccc(-c3c4ccccc4c(-c4ccc5ccccc5c4)c4ccccc34)cc2)cc1]

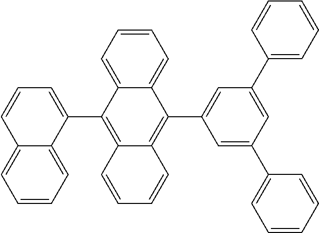 [SMILES: c1ccc(-c2cc(-c3ccccc3)cc(-c3c4ccccc4c(-c4cccc5ccccc45)c4ccccc34)c2)cc1]

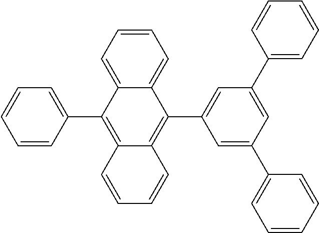 [SMILES: c1ccc(-c2cc(-c3ccccc3)cc(-c3c4ccccc4c(-c4ccccc4)c4ccccc34)c2)cc1]

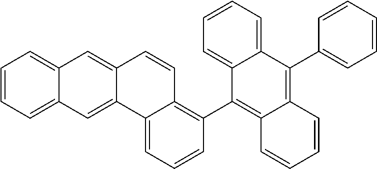 [SMILES: c1ccc(-c2c3ccccc3c(-c3cccc4c3ccc3cc5ccccc5cc34)c3ccccc23)cc1]

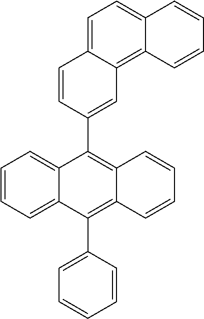 [SMILES: c1ccc(-c2c3ccccc3c(-c3ccc4ccc5ccccc5c4c3)c3ccccc23)cc1]

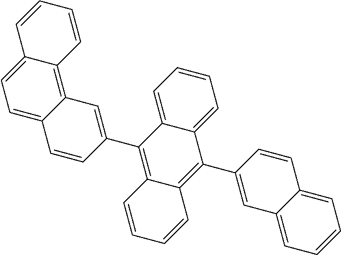 [SMILES: c1ccc2cc(-c3c4ccccc4c(-c4ccc5ccc6ccccc6c5c4)c4ccccc34)ccc2c1]

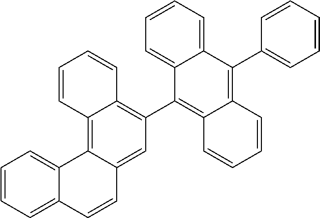 [SMILES: c1ccc(-c2c3ccccc3c(-c3cc4ccc5ccccc5c4c4ccccc34)c3ccccc23)cc1]

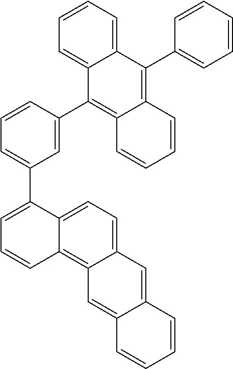 [SMILES: c1ccc(-c2c3ccccc3c(-c3cccc(-c4cccc5c4ccc4cc6ccccc6cc45)c3)c3ccccc23)cc1]

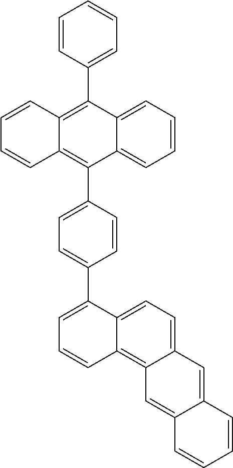 [SMILES: c1ccc(-c2c3ccccc3c(-c3ccc(-c4cccc5c4ccc4cc6ccccc6cc45)cc3)c3ccccc23)cc1]

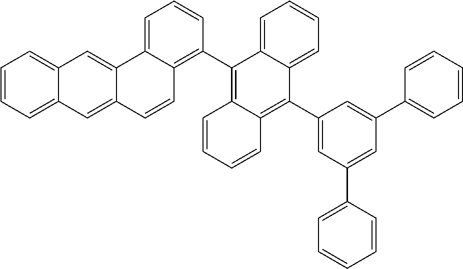 [SMILES: c1ccc(-c2cc(-c3ccccc3)cc(-c3c4ccccc4c(-c4cccc5c4ccc4cc6ccccc6cc45)c4ccccc34)c2)cc1]

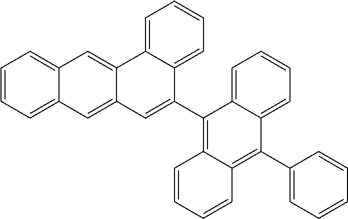 [SMILES: c1ccc(-c2c3ccccc3c(-c3cc4cc5ccccc5cc4c4ccccc34)c3ccccc23)cc1]

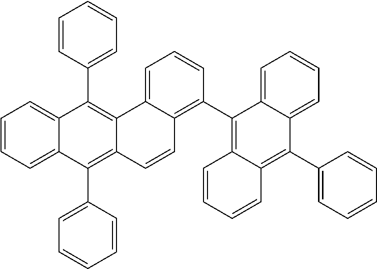 [SMILES: c1ccc(-c2c3ccccc3c(-c3cccc4c3ccc3c(-c5ccccc5)c5ccccc5c(-c5ccccc5)c34)c3ccccc23)cc1]

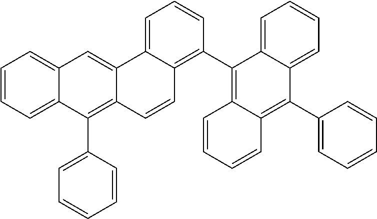 [SMILES: c1ccc(-c2c3ccccc3c(-c3cccc4c3ccc3c(-c5ccccc5)c5ccccc5cc34)c3ccccc23)cc1]

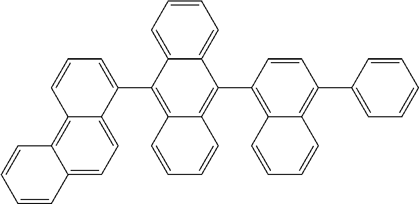 [SMILES: c1ccc(-c2ccc(-c3c4ccccc4c(-c4cccc5c4ccc4ccccc45)c4ccccc34)c3ccccc23)cc1]

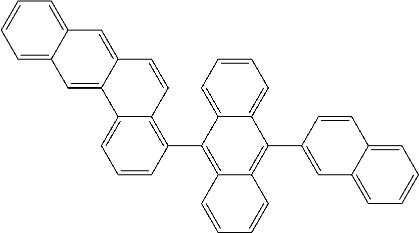 [SMILES: c1ccc2cc(-c3c4ccccc4c(-c4cccc5c4ccc4cc6ccccc6cc45)c4ccccc34)ccc2c1]

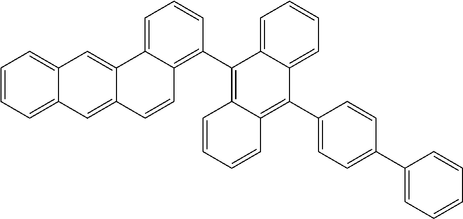 [SMILES: c1ccc(-c2ccc(-c3c4ccccc4c(-c4cccc5c4ccc4cc6ccccc6cc45)c4ccccc34)cc2)cc1]

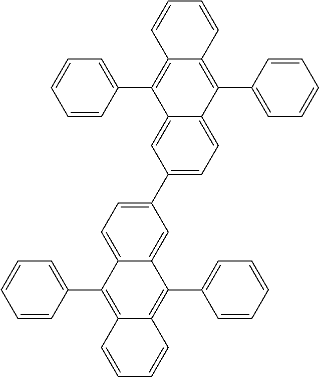 [SMILES: c1ccc(-c2c3ccccc3c(-c3ccccc3)c3cc(-c4ccc5c(-c6ccccc6)c6ccccc6c(-c6ccccc6)c5c4)ccc23)cc1]

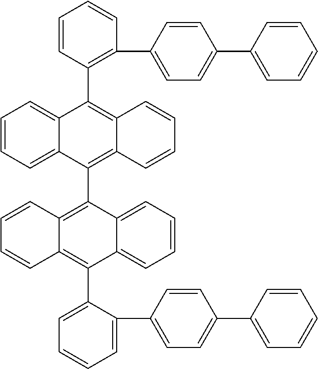 [SMILES: c1ccc(-c2ccc(-c3ccccc3-c3c4ccccc4c(-c4c5ccccc5c(-c5ccccc5-c5ccc(-c6ccccc6)cc5)c5ccccc45)c4ccccc34)cc2)cc1]

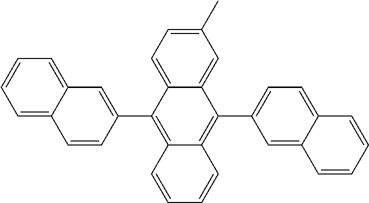 [SMILES: Cc1ccc2c(-c3ccc4ccccc4c3)c3ccccc3c(-c3ccc4ccccc4c3)c2c1]

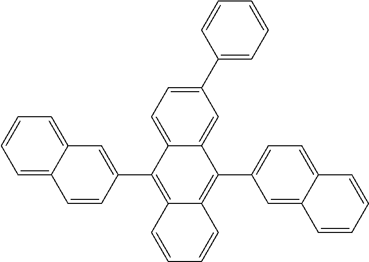 [SMILES: c1ccc(-c2ccc3c(-c4ccc5ccccc5c4)c4ccccc4c(-c4ccc5ccccc5c4)c3c2)cc1]

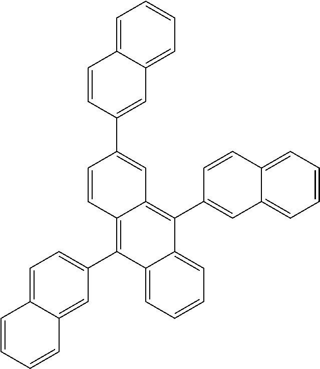 [SMILES: c1ccc2cc(-c3ccc4c(-c5ccc6ccccc6c5)c5ccccc5c(-c5ccc6ccccc6c5)c4c3)ccc2c1]

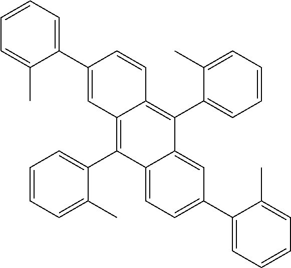 [SMILES: Cc1ccccc1-c1ccc2c(-c3ccccc3C)c3cc(-c4ccccc4C)ccc3c(-c3ccccc3C)c2c1]

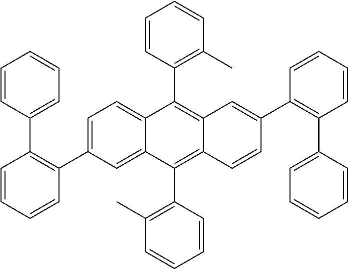 [SMILES: Cc1ccccc1-c1c2ccc(-c3ccccc3-c3ccccc3)cc2c(-c2ccccc2C)c2ccc(-c3ccccc3-c3ccccc3)cc12]

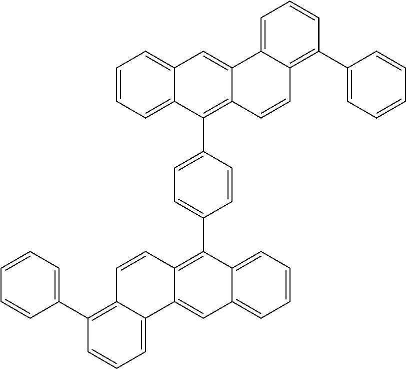 [SMILES: c1ccc(-c2cccc3c2ccc2c(-c4ccc(-c5c6ccccc6cc6c5ccc5c(-c7ccccc7)cccc56)cc4)c4ccccc4cc23)cc1]

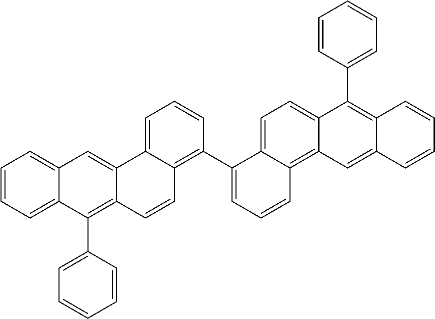 [SMILES: c1ccc(-c2c3ccccc3cc3c2ccc2c(-c4cccc5c4ccc4c(-c6ccccc6)c6ccccc6cc45)cccc23)cc1]

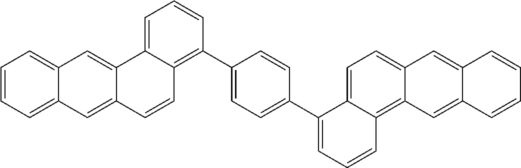 [SMILES: c1ccc2cc3c(ccc4c(-c5ccc(-c6cccc7c6ccc6cc8ccccc8cc67)cc5)cccc43)cc2c1]

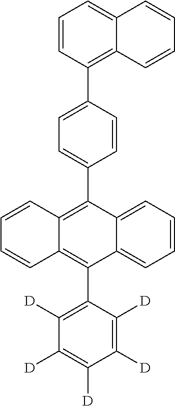 [SMILES: [2H]c1c([2H])c([2H])c(-c2c3ccccc3c(-c3ccc(-c4cccc5ccccc45)cc3)c3ccccc23)c([2H])c1[2H]]

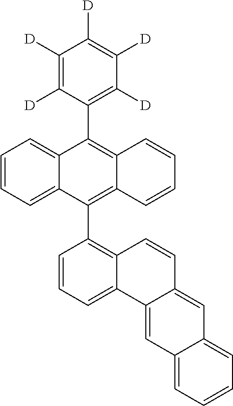 [SMILES: [2H]c1c([2H])c([2H])c(-c2c3ccccc3c(-c3cccc4c3ccc3cc5ccccc5cc34)c3ccccc23)c([2H])c1[2H]]

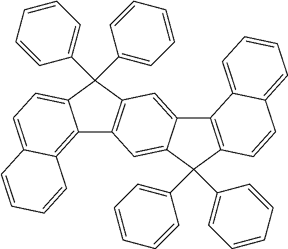 [SMILES: c1ccc(C2(c3ccccc3)c3cc4c(cc3-c3c2ccc2ccccc32)C(c2ccccc2)(c2ccccc2)c2ccc3ccccc3c2-4)cc1]

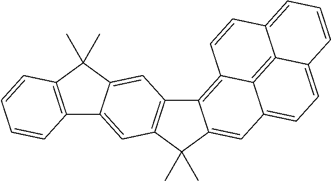 [SMILES: CC1(C)c2ccccc2-c2cc3c(cc21)-c1c(cc2ccc4cccc5ccc1c2c45)C3(C)C]

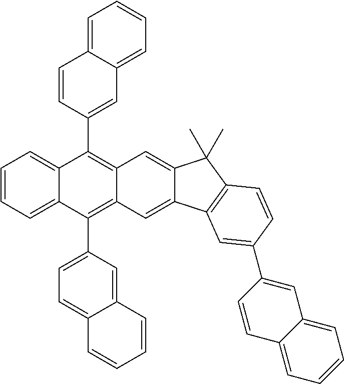 [SMILES: CC1(C)c2ccc(-c3ccc4ccccc4c3)cc2-c2cc3c(-c4ccc5ccccc5c4)c4ccccc4c(-c4ccc5ccccc5c4)c3cc21]

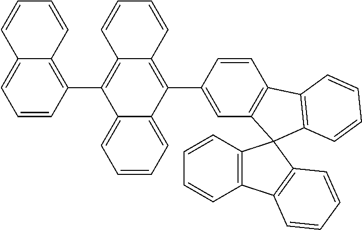 [SMILES: c1ccc2c(c1)-c1ccccc1C21c2ccccc2-c2ccc(-c3c4ccccc4c(-c4cccc5ccccc45)c4ccccc34)cc21]

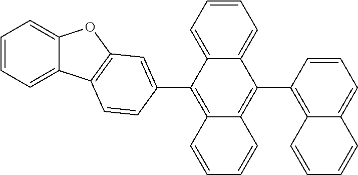 [SMILES: c1ccc2c(-c3c4ccccc4c(-c4ccc5c(c4)oc4ccccc45)c4ccccc34)cccc2c1]

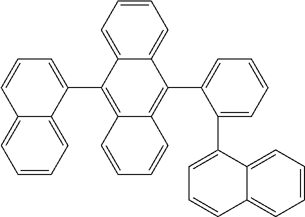 [SMILES: c1ccc(-c2c3ccccc3c(-c3cccc4ccccc34)c3ccccc23)c(-c2cccc3ccccc23)c1]

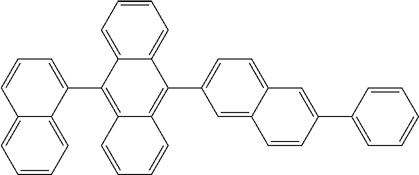 [SMILES: c1ccc(-c2ccc3cc(-c4c5ccccc5c(-c5cccc6ccccc56)c5ccccc45)ccc3c2)cc1]

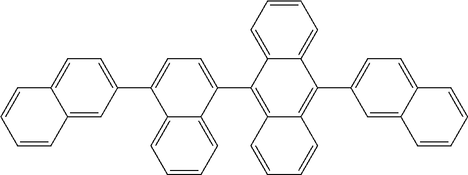 [SMILES: c1ccc2cc(-c3ccc(-c4c5ccccc5c(-c5ccc6ccccc6c5)c5ccccc45)c4ccccc34)ccc2c1]

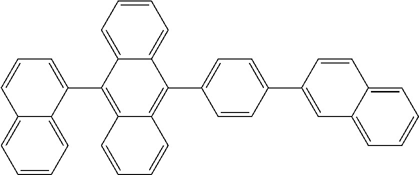 [SMILES: c1ccc2cc(-c3ccc(-c4c5ccccc5c(-c5cccc6ccccc56)c5ccccc45)cc3)ccc2c1]

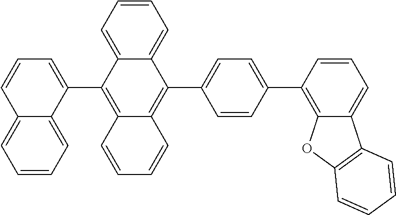 [SMILES: c1ccc2c(-c3c4ccccc4c(-c4ccc(-c5cccc6c5oc5ccccc56)cc4)c4ccccc34)cccc2c1]

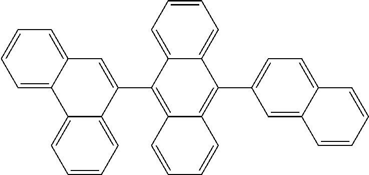 [SMILES: c1ccc2cc(-c3c4ccccc4c(-c4cc5ccccc5c5ccccc45)c4ccccc34)ccc2c1]

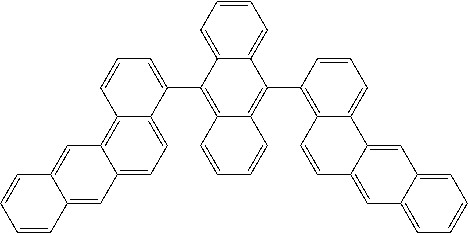 [SMILES: c1ccc2cc3c(ccc4c(-c5c6ccccc6c(-c6cccc7c6ccc6cc8ccccc8cc67)c6ccccc56)cccc43)cc2c1]

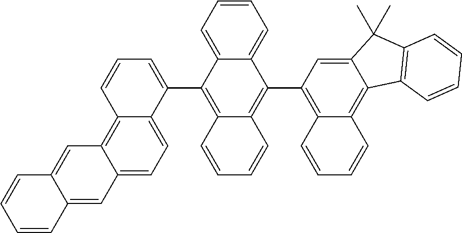 [SMILES: CC1(C)c2ccccc2-c2c1cc(-c1c3ccccc3c(-c3cccc4c3ccc3cc5ccccc5cc34)c3ccccc13)c1ccccc21]

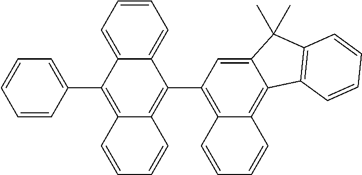 [SMILES: CC1(C)c2ccccc2-c2c1cc(-c1c3ccccc3c(-c3ccccc3)c3ccccc13)c1ccccc21]

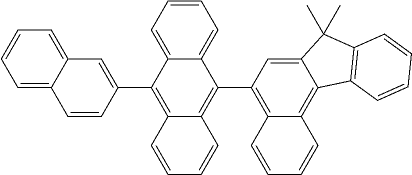 [SMILES: CC1(C)c2ccccc2-c2c1cc(-c1c3ccccc3c(-c3ccc4ccccc4c3)c3ccccc13)c1ccccc21]

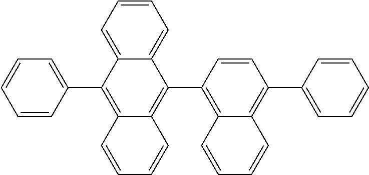 [SMILES: c1ccc(-c2ccc(-c3c4ccccc4c(-c4ccccc4)c4ccccc34)c3ccccc23)cc1]

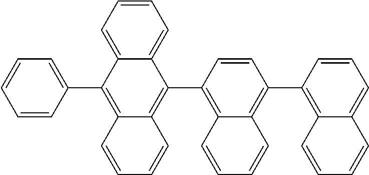 [SMILES: c1ccc(-c2c3ccccc3c(-c3ccc(-c4cccc5ccccc45)c4ccccc34)c3ccccc23)cc1]

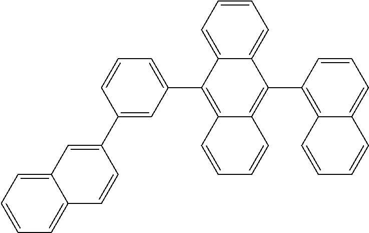 [SMILES: c1cc(-c2ccc3ccccc3c2)cc(-c2c3ccccc3c(-c3cccc4ccccc34)c3ccccc23)c1]

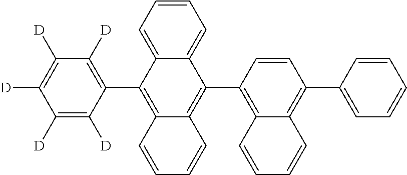 [SMILES: [2H]c1c([2H])c([2H])c(-c2c3ccccc3c(-c3ccc(-c4ccccc4)c4ccccc34)c3ccccc23)c([2H])c1[2H]]

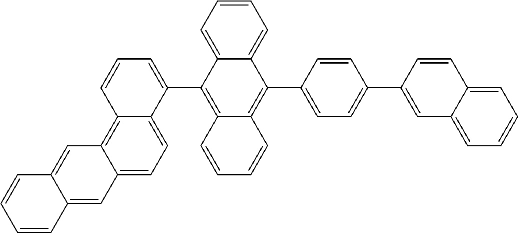 [SMILES: c1ccc2cc(-c3ccc(-c4c5ccccc5c(-c5cccc6c5ccc5cc7ccccc7cc56)c5ccccc45)cc3)ccc2c1]

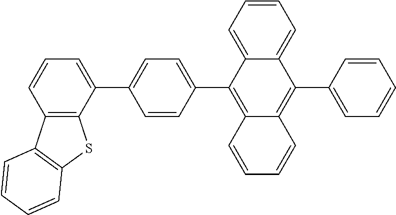 [SMILES: c1ccc(-c2c3ccccc3c(-c3ccc(-c4cccc5c4sc4ccccc45)cc3)c3ccccc23)cc1]

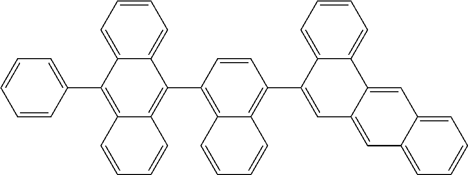 [SMILES: c1ccc(-c2c3ccccc3c(-c3ccc(-c4cc5cc6ccccc6cc5c5ccccc45)c4ccccc34)c3ccccc23)cc1]

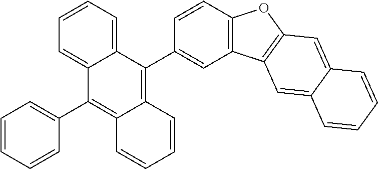 [SMILES: c1ccc(-c2c3ccccc3c(-c3ccc4oc5cc6ccccc6cc5c4c3)c3ccccc23)cc1]

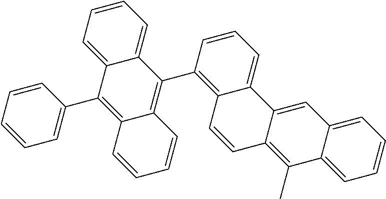 [SMILES: Cc1c2ccccc2cc2c1ccc1c(-c3c4ccccc4c(-c4ccccc4)c4ccccc34)cccc12]

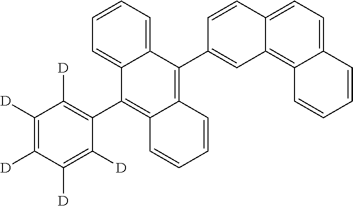 [SMILES: [2H]c1c([2H])c([2H])c(-c2c3ccccc3c(-c3ccc4ccc5ccccc5c4c3)c3ccccc23)c([2H])c1[2H]]

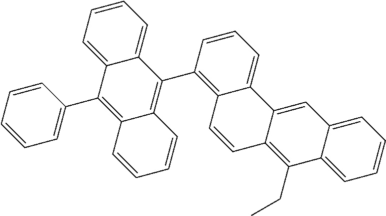 [SMILES: CCc1c2ccccc2cc2c1ccc1c(-c3c4ccccc4c(-c4ccccc4)c4ccccc34)cccc12]

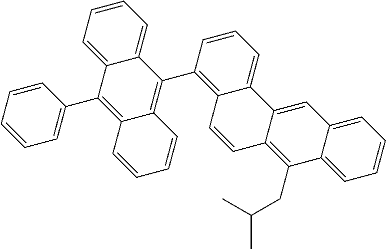 [SMILES: CC(C)Cc1c2ccccc2cc2c1ccc1c(-c3c4ccccc4c(-c4ccccc4)c4ccccc34)cccc12]

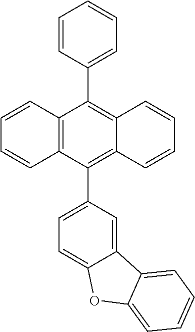 [SMILES: c1ccc(-c2c3ccccc3c(-c3ccc4oc5ccccc5c4c3)c3ccccc23)cc1]

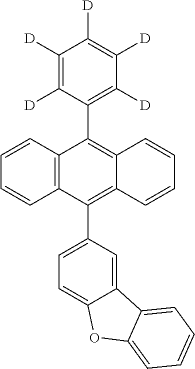 [SMILES: [2H]c1c([2H])c([2H])c(-c2c3ccccc3c(-c3ccc4oc5ccccc5c4c3)c3ccccc23)c([2H])c1[2H]]

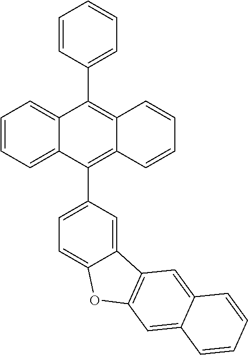 [SMILES: c1ccc(-c2c3ccccc3c(-c3ccc4oc5cc6ccccc6cc5c4c3)c3ccccc23)cc1]

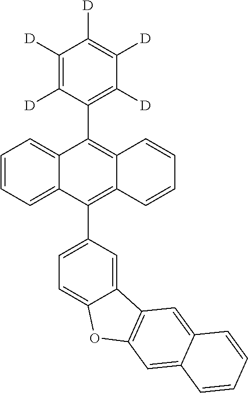 [SMILES: [2H]c1c([2H])c([2H])c(-c2c3ccccc3c(-c3ccc4oc5cc6ccccc6cc5c4c3)c3ccccc23)c([2H])c1[2H]]

On the other hand, the compounds according to the invention can also be employed as fluorescent emitting compounds. In this case, the suitable matrix materials for the compound of formula (1) used as a fluorescent emitting compound correspond to further compounds of formula (1) or to the preferred matrix materials described above.

The compounds according to the invention can also be employed in other layers, for example as hole-transport materials in a hole-injection or hole-transport layer or electron-blocking layer or as matrix materials in an emitting layer, preferably as matrix materials for phosphorescent emitters.

If the compound of the formula (1) is employed as hole-transport material in a hole-transport layer, a hole-injection layer or an electron-blocking layer, the compound can be employed as pure material, i.e. in a proportion of 100%, in the hole-transport layer, or it can be employed in combination with one or more further compounds. According to a preferred embodiment, the organic layer comprising the compound of the formula (1) then additionally comprises one or more p-dopants. The p-dopants employed in accordance with the present invention are preferably organic electron-acceptor compounds which are able to oxidise one or more of the other compounds of the mixture.

Particularly preferred embodiments of p-dopants are the compounds disclosed in WO 2011/073149, EP 1968131, EP 2276085, EP 2213662, EP 1722602, EP 2045848, DE 102007031220, U.S. Pat. Nos. 8,044,390, 8,057,712, WO 2009/003455, WO 2010/094378, WO 2011/120709, US 2010/0096600 and WO 2012/095143.

If the compound of the formula (1) is employed as matrix material in combination with a phosphorescent emitter in an emitting layer, the phosphorescent emitter is preferably selected from the classes and embodiments of phosphorescent emitters indicated below. Furthermore, one or more further matrix materials are preferably present in the emitting layer in this case.

So-called mixed-matrix systems of this type preferably comprise two or three different matrix materials, particularly preferably two different matrix materials. It is preferred here for one of the two materials to be a material having hole-transporting properties and for the other material to be a material having electron-transporting properties. The compound of the formula (1) is preferably the material having hole-transporting properties.

However, the desired electron-transporting and hole-transporting properties of the mixed-matrix components may also be combined mainly or completely in a single mixed-matrix component, where the further mixed-matrix component or components satisfy other functions. The two different matrix materials may be present here in a ratio of 1:50 to 1:1, preferably 1:20 to 1:1, particularly preferably 1:10 to 1:1 and very particularly preferably 1:4 to 1:1. Mixed-matrix systems are preferably employed in phosphorescent organic electroluminescent devices. Further details on mixed-matrix systems are contained, inter alia, in the application WO 2010/108579.

Particularly suitable matrix materials which can be used as matrix components of a mixed-matrix system in combination with the compounds according to the invention are selected from the preferred matrix materials for phosphorescent emitters indicated below or the preferred matrix materials for fluorescent emitters, depending on what type of emitter compound is employed in the mixed-matrix system.

Generally preferred classes of material for use as corresponding functional materials in the organic electroluminescent devices according to the invention are indicated below.

Suitable phosphorescent emitters are, in particular, compounds which emit light, preferably in the visible region, on suitable excitation and in addition contain at least one atom having an atomic number greater than 20, preferably greater than 38 and less than 84, particularly preferably greater than 56 and less than 80. The phosphorescent emitters used are preferably compounds which contain copper, molybdenum, tungsten, rhenium, ruthenium, osmium, rhodium, iridium, palladium, platinum, silver, gold or europium, in particular compounds which contain iridium, platinum or copper.

For the purposes of the present invention, all luminescent iridium, platinum or copper complexes are regarded as phosphorescent compounds.

Examples of the phosphorescent emitters described above are revealed by the applications WO 2000/70655, WO 2001/41512, WO 2002/02714, WO 2002/15645, EP 1191613, EP 1191612, EP 1191614, WO 2005/033244, WO 2005/019373 and US 2005/0258742. In general, all phosphorescent complexes as used in accordance with the prior art for phosphorescent OLEDs and as are known to the person skilled in the art in the area of organic electroluminescent devices are suitable for use in the devices according to the invention. The person skilled in the art will also be able to employ further phosphorescent complexes without inventive step in combination with the compounds according to the invention in OLEDs.

Preferred matrix materials for phosphorescent emitters are aromatic ketones, aromatic phosphine oxides or aromatic sulfoxides or sulfones, for example in accordance with WO 2004/013080, WO 2004/093207, WO 2006/005627 or WO 2010/006680, triarylamines, carbazole derivatives, for example CBP (N,N-biscarbazolylbiphenyl) or the carbazole derivatives disclosed in WO 2005/039246, US 2005/0069729, JP 2004/288381, EP 1205527 or WO 2008/086851, indolocarbazole derivatives, for example in accordance with WO 2007/063754 or WO 2008/056746, indenocarbazole derivatives, for example in accordance with WO 2010/136109, WO 2011/000455 or WO 2013/041176, azacarbazole derivatives, for example in accordance with EP 1617710, EP 1617711, EP 1731584, JP 2005/347160, bipolar matrix materials, for example in accordance with WO 2007/137725, silanes, for example in accordance with WO 2005/111172, azaboroles or boronic esters, for example in accordance with WO 2006/117052, triazine derivatives, for example in accordance with WO 2010/015306, WO 2007/063754 or WO 2008/056746, zinc complexes, for example in accordance with EP 652273 or WO 2009/062578, diazasilole or tetraazasilole derivatives, for example in accordance with WO 2010/054729, diazaphosphole derivatives, for example in accordance with WO 2010/054730, bridged carbazole derivatives, for example in accordance with US 2009/0136779, WO 2010/050778, WO 2011/042107, WO 2011/088877 or WO 2012/143080, triphenylene derivatives, for example in accordance with WO 2012/048781, or lactams, for example in accordance with WO 2011/116865 or WO 2011/137951.

Besides the compounds according to the invention, suitable charge-transport materials, as can be used in the hole-injection or hole-transport layer or electron-blocking layer or in the electron-transport layer of the electronic device according to the invention, are, for example, the compounds disclosed in Y. Shirota et al., Chem. Rev. 2007, 107(4), 953-1010, or other materials as are employed in these layers in accordance with the prior art.

Materials which can be used for the electron-transport layer are all materials as are used in accordance with the prior art as electron-transport materials in the electron-transport layer. Particularly suitable are aluminium complexes, for example Alq₃, zirconium complexes, for example Zrq₄, lithium complexes, for example LiQ, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives. Furthermore, suitable materials are derivatives of the above-mentioned compounds, as disclosed in JP 2000/053957, WO 2003/060956, WO 2004/028217, WO 2004/080975 and WO 2010/072300.

Preferred hole-transport materials which can be used in a hole-transport, hole-injection or electron-blocking layer in the electroluminescent device according to the invention are indenofluorenamine derivatives (for example in accordance with WO 06/122630 or WO 06/100896), the amine derivatives disclosed in EP 1661888, hexaazatriphenylene derivatives (for example in accordance with WO 01/049806), amine derivatives containing condensed aromatic rings (for example in accordance with U.S. Pat. No. 5,061,569), the amine derivatives disclosed in WO 95/09147, monobenzoindenofluorenamines (for example in accordance with WO 08/006449), dibenzoindenofluorenamines (for example in accordance with WO 07/140847), spirobifluorenamines (for example in accordance with WO 2012/034627 or WO 2013/120577), fluorenamines (for example in accordance with the as applications EP 2875092, EP 2875699 and EP 2875004), spirodibenzopyranamines (for example in accordance with WO 2013/083216) and dihydroacridine derivatives (for example in accordance with WO 2012/150001). The compounds according to the invention can also be used as hole-transport materials.

The cathode of the organic electroluminescent device preferably comprises metals having a low work function, metal alloys or multilayered structures comprising various metals, such as, for example, alkaline-earth metals, alkali metals, main-group metals or lanthanoids (for example Ca, Ba, Mg, Al, In, Mg, Yb, Sm, etc.). Also suitable are alloys comprising an alkali metal or alkaline-earth metal and silver, for example an alloy comprising magnesium and silver. In the case of multilayered structures, further metals which have a relatively high work function, such as, for example, Ag or Al, can also be used in addition to the said metals, in which case combinations of the metals, such as, for example, Ca/Ag, Mg/Ag or Ag/Ag, are generally used. It may also be preferred to introduce a thin interlayer of a material having a high dielectric constant between a metallic cathode and the organic semiconductor. Suitable for this purpose are, for example, alkali metal fluorides or alkaline-earth metal fluorides, but also the corresponding oxides or carbonates (for example LiF, Li₂O, BaF₂, MgO, NaF, CsF, Cs₂CO₃, etc.). Furthermore, lithium quinolinate (LiQ) can be used for this purpose. The layer thickness of this layer is preferably between 0.5 and 5 nm.

The anode preferably comprises materials having a high work function. The anode preferably has a work function of greater than 4.5 eV vs. vacuum. Suitable for this purpose are on the one hand metals having a high redox potential, such as, for example, Ag, Pt or Au. On the other hand, metal/metal oxide electrodes (for example AI/Ni/NiOx, AI/PtOx) may also be preferred. For some applications, at least one of the electrodes must be transparent or partially transparent in order to facilitate either irradiation of the organic material (organic solar cells) or the coupling-out of light (OLEDs, O-lasers). Preferred anode materials here are conductive mixed metal oxides. Particular preference is given to indium tin oxide (ITO) or indium zinc oxide (IZO). Preference is furthermore given to conductive, doped organic materials, in particular conductive doped polymers.

The device is appropriately (depending on the application) structured, provided with contacts and finally sealed, since the lifetime of the devices according to the invention is shortened in the presence of water and/or air.

In a preferred embodiment, the organic electroluminescent device according to the invention is characterised in that one or more layers are coated by means of a sublimation process, in which the materials are applied by vapour deposition in vacuum sublimation units at an initial pressure of less than 10⁻⁵ mbar, preferably less than 10⁻⁶ mbar. However, it is also possible here for the initial pressure to be even lower, for example less than 10⁻⁷ mbar.

Preference is likewise given to an organic electroluminescent device, characterised in that one or more layers are coated by means of the OVPD (organic vapour phase deposition) process or with the aid of carrier-gas sublimation, in which the materials are applied at a pressure of between 10⁻⁵ mbar and 1 bar. A special case of this process is the OVJP (organic vapour jet printing) process, in which the materials are applied directly through a nozzle and are thus structured (for example M. S. Arnold et al., Appl. Phys. Lett. 2008, 92, 053301).

Preference is furthermore given to an organic electroluminescent device, characterised in that one or more layers are produced from solution, such as, for example, by spin coating, or by means of any desired printing process, such as, for example, screen printing, flexographic printing, nozzle printing or offset printing, but particularly preferably LITI (light induced thermal imaging, thermal transfer printing) or ink-jet printing. Soluble compounds of the formula (I) are necessary for this purpose. High solubility can be achieved through suitable substitution of the compounds.

Also possible are hybrid processes, in which, for example, one or more layers are applied from solution and one or more further layers are applied by vapour deposition. Thus, it is possible, for example, to apply the emitting layer from solution and to apply the electron-transport layer by vapour deposition. These processes are generally known to the person skilled in the art and can be applied by him without inventive step to organic electroluminescent devices comprising the compounds according to the invention.

In accordance with the invention, the electronic devices comprising one or more compounds according to the invention can be employed in displays, as light sources in lighting applications and as light sources in medical and/or cosmetic applications (for example light therapy).

The invention will now be explained in greater detail by the following examples, without wishing to restrict it thereby.

### A) SYNTHESES EXAMPLES

### A-1) Part 1

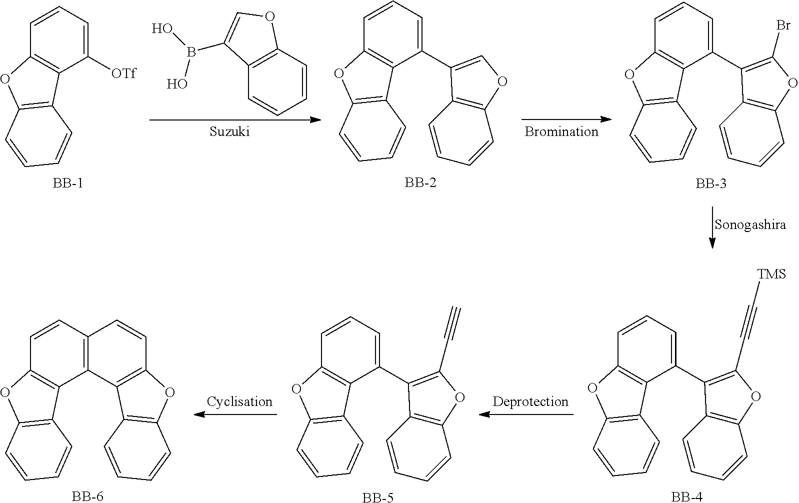

### Synthesis of BB-2

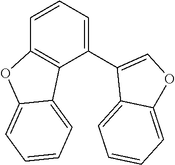 [SMILES: c1ccc2c(-c3cccc4oc5ccccc5c34)coc2c1]

Under an argon atmosphere, an oven dried flask is equipped with a magnetic stir bar, 1-((trifluoromethyl)sulfonyl)dibenzo[b,d]furane (20.0 g, 63.2 mmol, 1.0 equiv.), benzofurane-3-ylboronic acid (11.3 g, 69.6 mmol, 1.1 equiv.), potassium phosphate (33.6 g, 158.1 mmol, 2.5 equiv.), palladium acetate (0.3 g, 1.3 mmol, 0.02 equiv.) and XPhos (1.2 g, 2.5 mmol, 0.04 equiv.). THF (400 mL) and water (100 mL) are added and the reaction is refluxed overnight. The raw product is purified by column chromatography. The desired product is isolated as a colorless oil (15.0 g, 52.8 mmol, 83.3%).

### Synthesis of BB-3

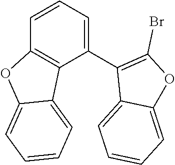 [SMILES: Brc1oc2ccccc2c1-c1cccc2oc3ccccc3c12]

An oven dried flask is equipped with BB-2 (15.0 g, 52.7 mmol, 1.0 equiv.) in DCM (150 mL). N-bromosuccinimide (9.4 g, 52.7 mmol, 1.0 equiv.) is added and the resulting mixture is stirred for overnight at rt. The raw product is purified by filtration over AlOx. The desired product is isolated as colorless oil (16.2 g, 44.3 mmol, 84.1%).

### Synthesis of BB-4

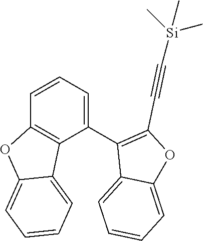 [SMILES: C[Si](C)(C)C#Cc1oc2ccccc2c1-c1cccc2oc3ccccc3c12]

Under an argon atmosphere, an oven dried flask is equipped with a magnetic stir bar, BB-3, copper iodide (0.3 g, 1.3 mmol, 0.03 equiv.), bis(triphenylphosphin)palladium(II)chlorid (0.6 g, 0.9 mmol, 0.02 equiv.), and trimethylsilylacetylene (18.9 mL, 133.8 mmol, 3.0 equiv.). Triethylamine (500 mL) is added and the reaction mixture is refluxed overnight. The raw product is purified by column chromatography. The desired product is isolated as a white solid (13.6 g, 35.7 mmol, 80.1%).

### Synthesis of BB-5

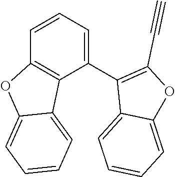 [SMILES: C#Cc1oc2ccccc2c1-c1cccc2oc3ccccc3c12]

An oven dried flask is equipped with a magnetic stir bar, BB-4 (10.0 g, 26.3 mmol, 1.0 equiv.), potassium carbonate (0.7 g, 5.3 mmol, 0.2 equiv.). Methanol (100 mL) is added and the reaction mixture is stirred for 1 h at rt. The solvent is removed under reduced pressure. The residue is taken up with DCM (100 mL) and is washed twice with water (2×50 mL). The organic phase is concentrated under reduce pressure. The desired product is obtained as white solid (8.1 g, 26.3 mmol, 100%).

### Synthesis of BB-6

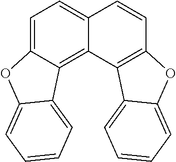 [SMILES: c1ccc2c(c1)oc1ccc3ccc4oc5ccccc5c4c3c12]

Under an argon atmosphere, an oven dried flask is charged with BB-5 (8.1 g, 26.0 mmol, 1.0 equiv.), platinum chloride (690 mg, 2.6 mmol, 0.1 equiv.). Toluene (500 mL) is added and the reaction mixture is refluxed overnight. The raw product is purified by column chromatography. The desired product is isolated as white solid (3.1 g, 10.0 mmol, 38.7%).

### A-2) Part 2

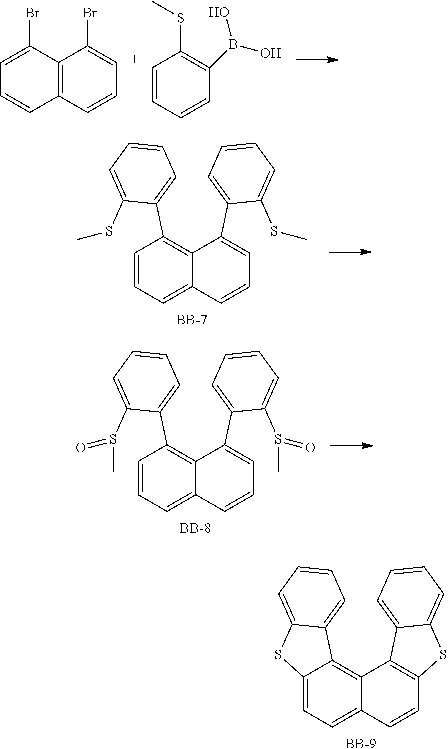

### Synthesis of BB-7

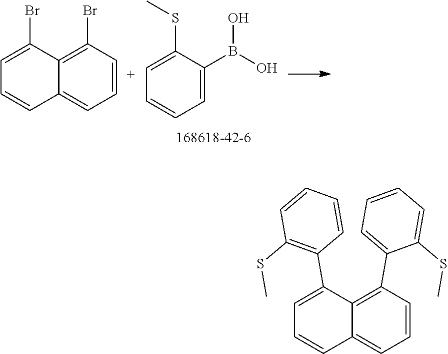

5 g (17.4 mmol) 1,8-dibromnapthalene, 7 g (43.7 mmol) [2-(Methylsulfanylphenyl] boronic acid and 28 g (87 mmol) cesium carbonate are mixed in 200 ml water and 200 ml N,N-Dimethylformamide. 0.71 g (1.7 mmol) SPhos and 1,68 g (1.7 mmol) Pd₂(dba)₃ are added and the mixture is refluxed for 17 h. After cooling down to room temperature the organic phase is separated and washed with water (3×200 ml) and with 200 ml brine. Afterward it is dried over magnesium sulfate and reduced under reduced pressure to give a gray residue, which is further purified by crystallization out of heptane.

Yield: 5.9 g, (15.9 mmol; 91%)

### Synthesis of BB-8

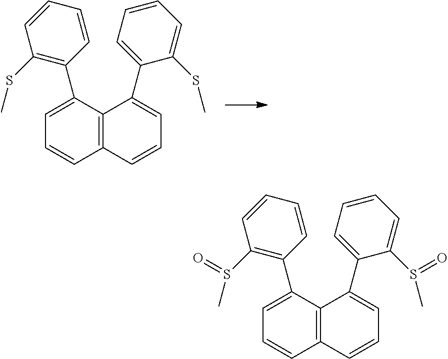

To 30 g (80 mmol) BB-7 60 ml acetic acid are added and cooled down to 0° C. 18.2 mL (160 mmol) of a 30% H₂O₂-solution are added dropwise and the mixture is stirred for 16 hours. A solution of Na₂SO₃ is added, the organic phase is separated and solvents are removed under reduced pressure.

Yield: 26 g (65 mmol; 80%)

### Synthesis of BB-9

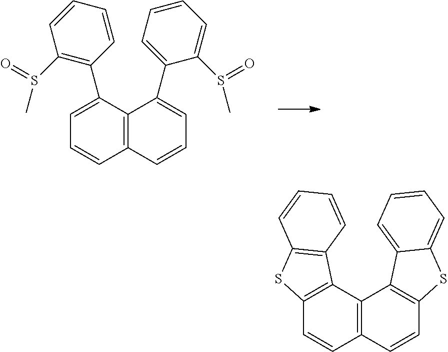 [SMILES: CS(=O)c1ccccc1-c1cccc2cccc(-c3ccccc3S(C)=O)c12 ; c1ccc2c(c1)sc1ccc3ccc4sc5ccccc5c4c3c12]

A mixture of 133 g (230 mmol) BB-8 and 200 ml triflic acid is stirred at 50° C. for 3 days. Afterwards 600 g (2.9 mol) potassium carbonate in 3 l water are added dropwise and stirred at 75° C. for 5 h. 500 ml toluene are added and the mixture is stirred at room temperature overnight. The organic phase is separated and reduced under reduced pressure. The residue was further purified by column chromatography (heptane/DCM)

Yield: 39 g (117 mmol, 52%)

### A-3) Part 3

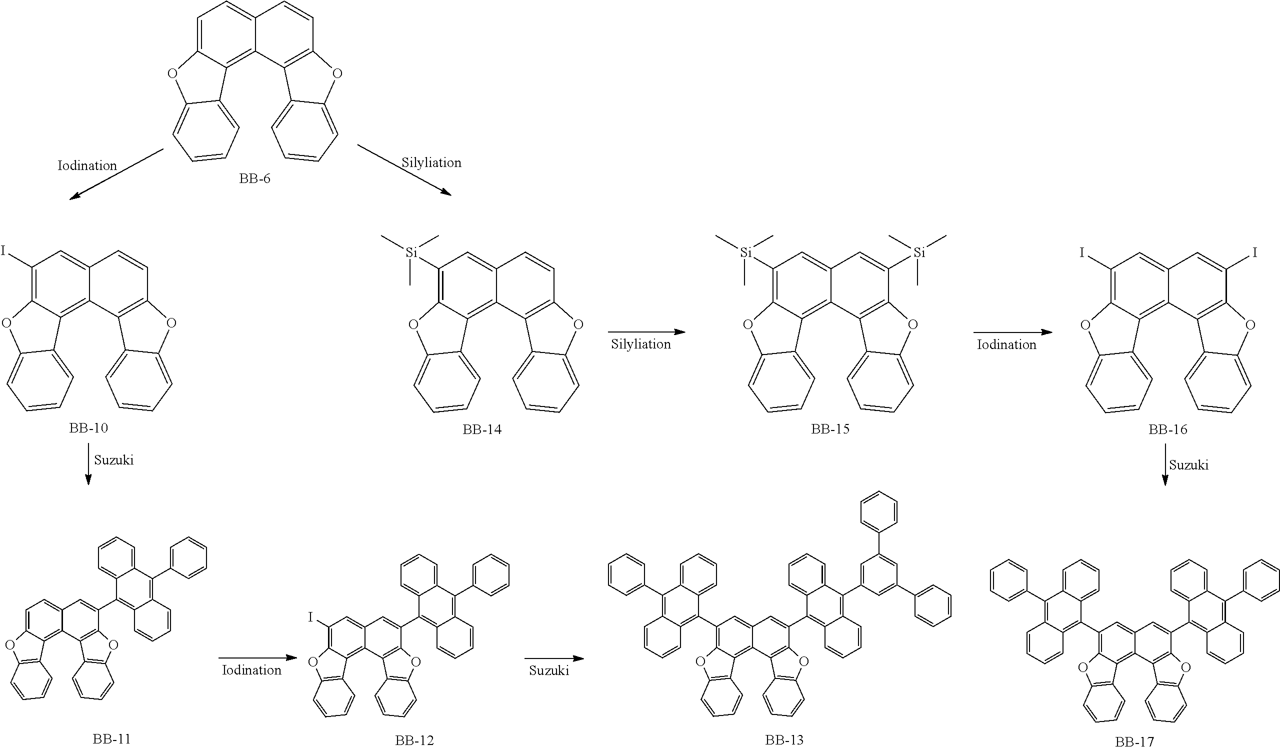

### Synthesis of BB-10

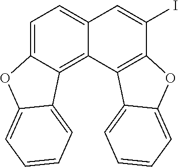 [SMILES: Ic1cc2ccc3oc4ccccc4c3c2c2c1oc1ccccc12]

Under an argon atmosphere, an oven dried flask is equipped with a magnetic stir bar, BB-6 (10.0 g, 32.4 mmol, 1.0 equiv.). THF (10 mL) is added and the reaction mixture is cooled to −78° C. n-BuLi (2.5 M in hexane, 20 mL, 48.7 mmol, 1.5 equiv.) is added slowly. The reaction mixture is stirred for 1 h at −78° C. Iodine (13.2 g, 52.0 mmol, 1.5 equiv.) dissolved in THF (20 mL) is added. The reaction mixture is warmed to room temperature overnight. The reaction mixture is diluted with ethyl acetate (1000 mL). Excess of iodine is quenched by the addition of saturated sodium thiosulfate solution (200 mL). The organic phase is separated. The solvent is removed under reduced pressure. The raw product is purified by column chromatography. The desired product is isolated as white solid (13.5 g, 31.1 mmol, 95.9%).

Following compounds can be synthesized in analogous manner:

| | Starting material | Product |
|---|---|---|
| BB-10-b | BB-9 | 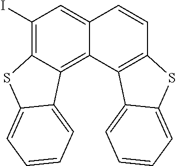 [SMILES: Ic1cc2ccc3sc4ccccc4c3c2c2c1sc1ccccc12] |
| BB-10-c* | BB-9 | 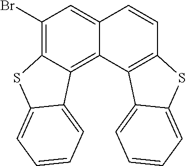 [SMILES: Brc1cc2ccc3sc4ccccc4c3c2c2c1sc1ccccc12] |
| BB-10-d* | BB-6 | 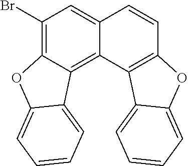 [SMILES: Brc1cc2ccc3oc4ccccc4c3c2c2c1oc1ccccc12] |

*Bromine is used instead of iodine

### Synthesis of BB-11

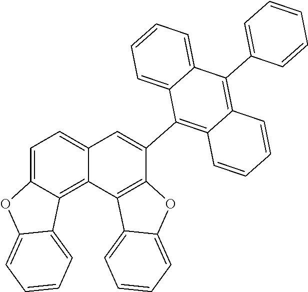 [SMILES: c1ccc(-c2c3ccccc3c(-c3cc4ccc5oc6ccccc6c5c4c4c3oc3ccccc34)c3ccccc23)cc1]

Under an argon atmosphere, an oven dried flask is equipped with a magnetic stir bar, BB-10, (13.0 g, 28.4 mmol, 1.0 equiv.), (10-phenyl-9-anthryl) boronic acid (25.4 g, 85.1 mmol, 3.0 equiv.), tris(dibenzylideneacetone) dipalladium (1.3 g, 1.4 mmol, 0.05 equiv.), SPhos (1.16 g, 2.8 mmol, 0.1 equiv.) and potassium fluoride (4.1 g, 70.9 mmol, 2.5 equiv.). Toluene (150 mL), 1,4-dioxane (150 mL) and water (150 mL) is added and the mixture is refluxed overnight. The raw product is purified by column chromatography and sublimation. The desired product is isolated as white solid (4.0 g, 7.1 mmol, 25.1%).

Following compounds can be synthesized in analogous manner:

| | Starting | | |
|---|---|---|---|
| | material | Starting material | Product |
| BB-11-b | BB-10-b | 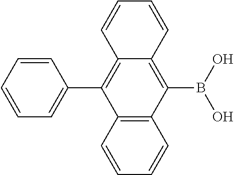 [SMILES: OB(O)c1c2ccccc2c(-c2ccccc2)c2ccccc12] | 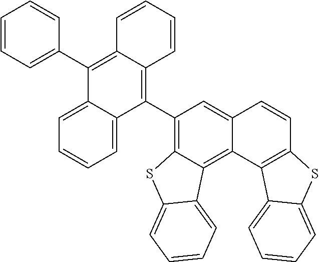 [SMILES: c1ccc(-c2c3ccccc3c(-c3cc4ccc5sc6ccccc6c5c4c4c3sc3ccccc34)c3ccccc23)cc1] |
| BB-11-c | BB-10-b | 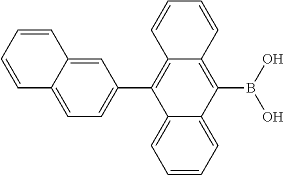 [SMILES: OB(O)c1c2ccccc2c(-c2ccc3ccccc3c2)c2ccccc12] | 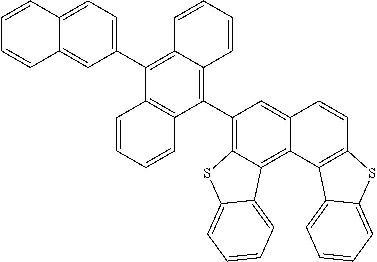 [SMILES: c1ccc2cc(-c3c4ccccc4c(-c4cc5ccc6sc7ccccc7c6c5c5c4sc4ccccc45)c4ccccc34)ccc2c1] |
| BB-11-d | BB-10 | 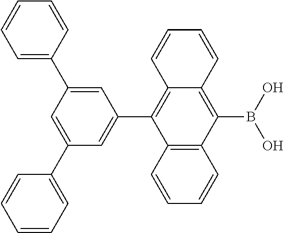 [SMILES: OB(O)c1c2ccccc2c(-c2cc(-c3ccccc3)cc(-c3ccccc3)c2)c2ccccc12] | 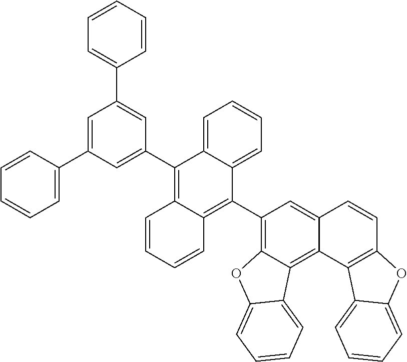 [SMILES: c1ccc(-c2cc(-c3ccccc3)cc(-c3c4ccccc4c(-c4cc5ccc6oc7ccccc7c6c5c5c4oc4ccccc45)c4ccccc34)c2)cc1] |
| BB-11-e | BB-10 | 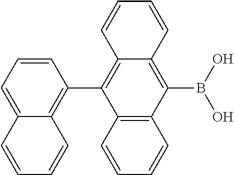 [SMILES: OB(O)c1c2ccccc2c(-c2cccc3ccccc23)c2ccccc12] | 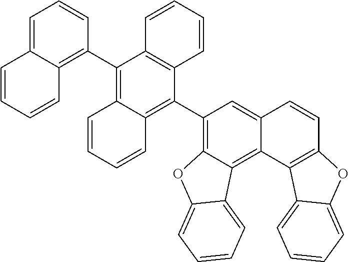 [SMILES: c1ccc2c(-c3c4ccccc4c(-c4cc5ccc6oc7ccccc7c6c5c5c4oc4ccccc45)c4ccccc34)cccc2c1] |
| BB-11-f | BB-10 | 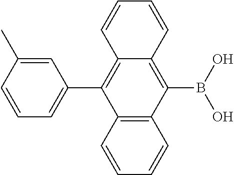 [SMILES: Cc1cccc(-c2c3ccccc3c(B(O)O)c3ccccc23)c1] | 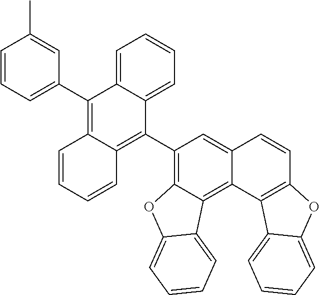 [SMILES: Cc1cccc(-c2c3ccccc3c(-c3cc4ccc5oc6ccccc6c5c4c4c3oc3ccccc34)c3ccccc23)c1] |
| BB-11-g | BB-10 | 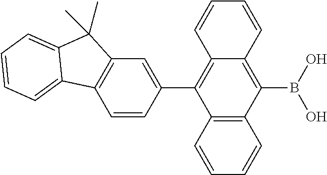 [SMILES: CC1(C)c2ccccc2-c2ccc(-c3c4ccccc4c(B(O)O)c4ccccc34)cc21] | 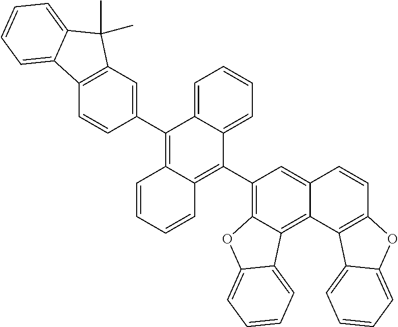 [SMILES: CC1(C)c2ccccc2-c2ccc(-c3c4ccccc4c(-c4cc5ccc6oc7ccccc7c6c5c5c4oc4ccccc45)c4ccccc34)cc21] |
| BB-11-h | BB-10-b | 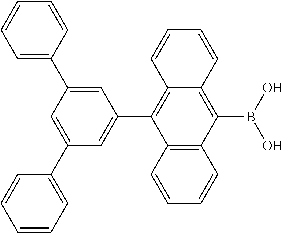 [SMILES: OB(O)c1c2ccccc2c(-c2cc(-c3ccccc3)cc(-c3ccccc3)c2)c2ccccc12] | 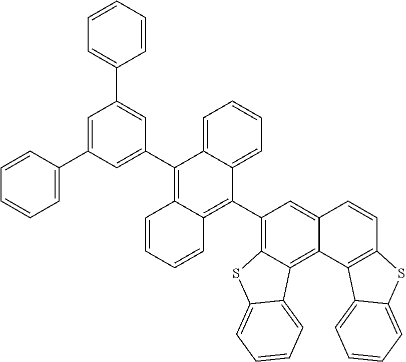 [SMILES: c1ccc(-c2cc(-c3ccccc3)cc(-c3c4ccccc4c(-c4cc5ccc6sc7ccccc7c6c5c5c4sc4ccccc45)c4ccccc34)c2)cc1] |
| BB-11-i | BB-10 | 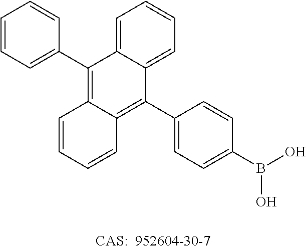 [SMILES: OB(O)c1ccc(-c2c3ccccc3c(-c3ccccc3)c3ccccc23)cc1] | 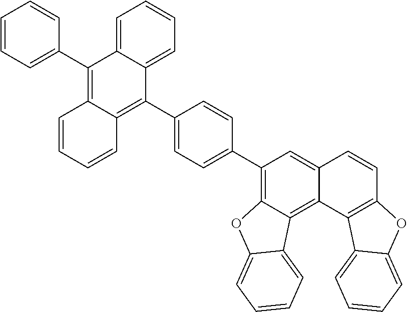 [SMILES: c1ccc(-c2c3ccccc3c(-c3ccc(-c4cc5ccc6oc7ccccc7c6c5c5c4oc4ccccc45)cc3)c3ccccc23)cc1] |
| BB-11-j | BB-10 | 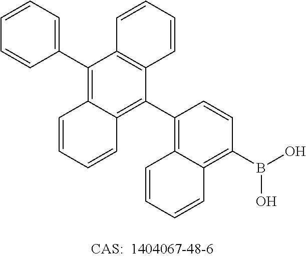 [SMILES: OB(O)c1ccc(-c2c3ccccc3c(-c3ccccc3)c3ccccc23)c2ccccc12] | 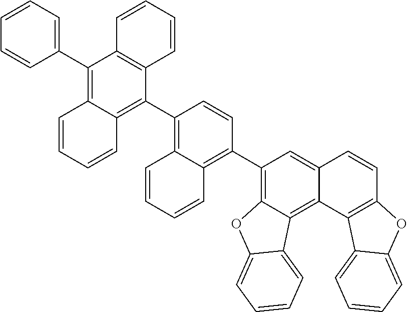 [SMILES: c1ccc(-c2c3ccccc3c(-c3ccc(-c4cc5ccc6oc7ccccc7c6c5c5c4oc4ccccc45)c4ccccc34)c3ccccc23)cc1] |
| BB-11-k | BB-10-b | 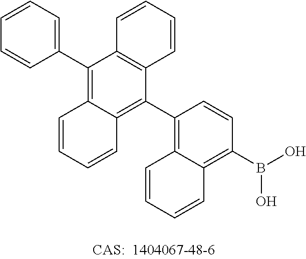 [SMILES: OB(O)c1ccc(-c2c3ccccc3c(-c3ccccc3)c3ccccc23)c2ccccc12] | 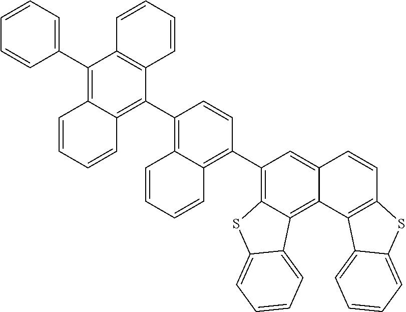 [SMILES: c1ccc(-c2c3ccccc3c(-c3ccc(-c4cc5ccc6sc7ccccc7c6c5c5c4sc4ccccc45)c4ccccc34)c3ccccc23)cc1] |
| BB-11-l | | 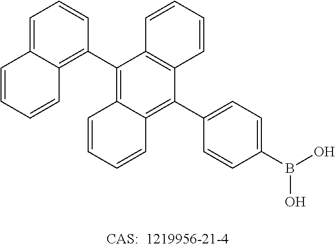 [SMILES: OB(O)c1ccc(-c2c3ccccc3c(-c3cccc4ccccc34)c3ccccc23)cc1] | 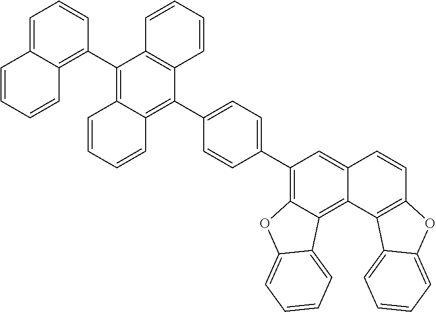 [SMILES: c1ccc2c(-c3c4ccccc4c(-c4ccc(-c5cc6ccc7oc8ccccc8c7c6c6c5oc5ccccc56)cc4)c4ccccc34)cccc2c1] |

### Synthesis of BB-12

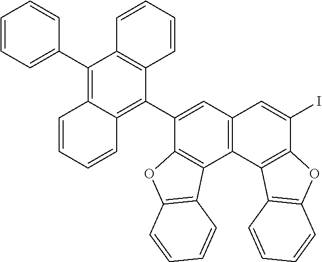 [SMILES: Ic1cc2cc(-c3c4ccccc4c(-c4ccccc4)c4ccccc34)c3oc4ccccc4c3c2c2c1oc1ccccc12]

Under an argon atmosphere, an oven dried flask is equipped with a magnetic stir bar, BB-11 (15.0 g, 26.8 mmol, 1.0 equiv.). THF (200 mL) is added and the reaction mixture is cooled to −78° C. n-BuLi (2.5 M in hexane, 21 mL, 53.5 mmol, 2.0 equiv.) is added slowly. The reaction mixture is stirred for 3 h at −78 OC. Iodine (17.0 g, 66.9 mmol, 2.5 equiv.) dissolved in THF (30 mL) is added. The reaction mixture is warmed to rt overnight. The reaction mixture is diluted with ethyl acetate (1000 mL). Excess of iodine is quenched by the addition of saturated sodium thiosulfate solution (200 mL). The organic phase is separated. The solvent is removed under reduced pressure. The raw product is purified by column chromatography. The desired product is isolated as white solid (15.0 g, 21.9 mmol, 81.7%).

Following compounds can be synthesized in analogous manner:

| | Starting material | Product |
|---|---|---|
| BB-12-b | BB-11-b | 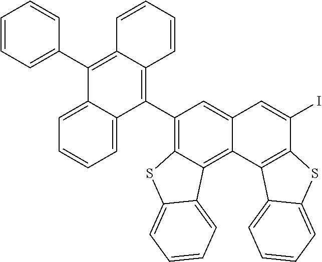 [SMILES: Ic1cc2cc(-c3c4ccccc4c(-c4ccccc4)c4ccccc34)c3sc4ccccc4c3c2c2c1sc1ccccc12] |
| BB-12-c | BB-11-c | 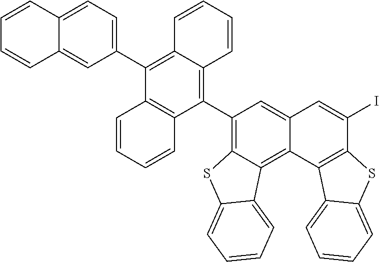 [SMILES: Ic1cc2cc(-c3c4ccccc4c(-c4ccc5ccccc5c4)c4ccccc34)c3sc4ccccc4c3c2c2c1sc1ccccc12] |
| BB-12-d | BB-11-d | 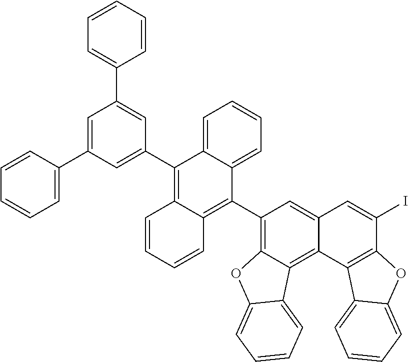 [SMILES: Ic1cc2cc(-c3c4ccccc4c(-c4cc(-c5ccccc5)cc(-c5ccccc5)c4)c4ccccc34)c3oc4ccccc4c3c2c2c1oc1ccccc12] |
| BB-12-e | BB-11-e | 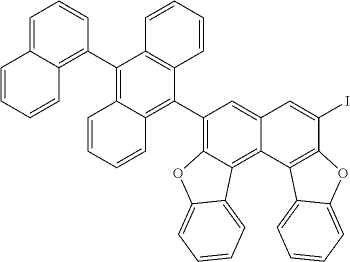 [SMILES: Ic1cc2cc(-c3c4ccccc4c(-c4cccc5ccccc45)c4ccccc34)c3oc4ccccc4c3c2c2c1oc1ccccc12] |
| BB-12-f | BB-11-f | 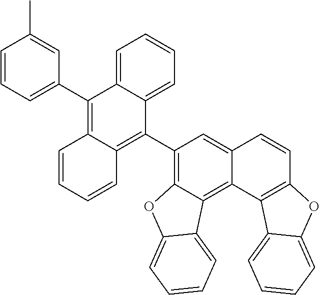 [SMILES: Cc1cccc(-c2c3ccccc3c(-c3cc4ccc5oc6ccccc6c5c4c4c3oc3ccccc34)c3ccccc23)c1] |

### Synthesis of BB-13

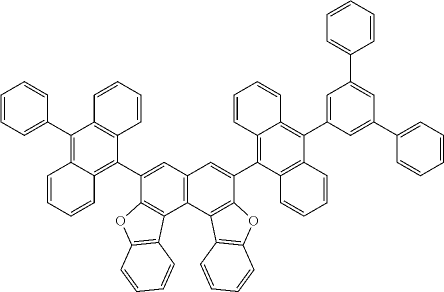 [SMILES: c1ccc(-c2cc(-c3ccccc3)cc(-c3c4ccccc4c(-c4cc5cc(-c6c7ccccc7c(-c7ccccc7)c7ccccc67)c6oc7ccccc7c6c5c5c4oc4ccccc45)c4ccccc34)c2)cc1]

Under an argon atmosphere, an oven dried flask is equipped with a magnetic stir bar, 1-Iodo-BB-12 (14.5 g, 21.1 mmol, 1.0 equiv.), 10-Phenyl-9-anthranyl-boronic acid (28.5 g, 63.4 mmol, 3.0 equiv.), potassium fluoride (73.6 g, 126.7, mmol, 6.0 equiv.) and (2-Dicyclohexylphosphino-2′,6′-dimethoxybiphenyl) [2-(2′-amino-1,1′-biphenyl)]palladium(II) methanesulfonate (1.65 g, 2.11 mmol, 0.1 equiv.). Toluene (300 mL), 1.4-dioxane (300 mL) and water (300 mL) is added and the mixture is refluxed overnight. The raw product is purified by column chromatography. The desired product is isolated as white solid (6.8 g, 7.05 mmol, 33.4%).

Following compounds can be synthesized in analogous manner:

| | Starting | | |
|---|---|---|---|
| | material | Starting material | Product |
| BB-13-b | BB-12-b | 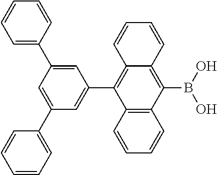 [SMILES: OB(O)c1c2ccccc2c(-c2cc(-c3ccccc3)cc(-c3ccccc3)c2)c2ccccc12] | 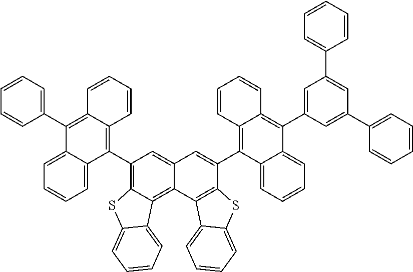 [SMILES: c1ccc(-c2cc(-c3ccccc3)cc(-c3c4ccccc4c(-c4cc5cc(-c6c7ccccc7c(-c7ccccc7)c7ccccc67)c6sc7ccccc7c6c5c5c4sc4ccccc45)c4ccccc34)c2)cc1] |
| BB-13-c | BB-12-c | 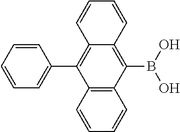 [SMILES: OB(O)c1c2ccccc2c(-c2ccccc2)c2ccccc12] | 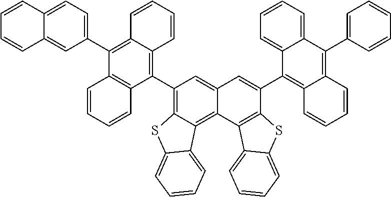 [SMILES: c1ccc(-c2c3ccccc3c(-c3cc4cc(-c5c6ccccc6c(-c6ccc7ccccc7c6)c6ccccc56)c5sc6ccccc6c5c4c4c3sc3ccccc34)c3ccccc23)cc1] |
| BB-13-d | BB-12-d | 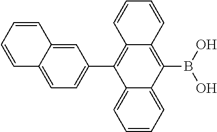 [SMILES: OB(O)c1c2ccccc2c(-c2ccc3ccccc3c2)c2ccccc12] | 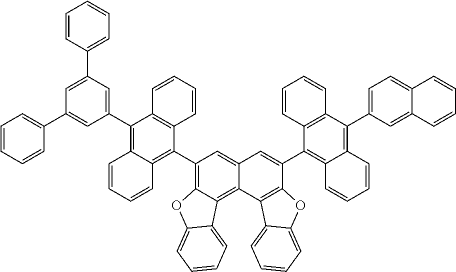 [SMILES: c1ccc(-c2cc(-c3ccccc3)cc(-c3c4ccccc4c(-c4cc5cc(-c6c7ccccc7c(-c7ccc8ccccc8c7)c7ccccc67)c6oc7ccccc7c6c5c5c4oc4ccccc45)c4ccccc34)c2)cc1] |
| BB-13-e | BB-12-e | 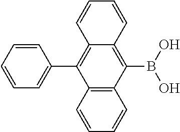 [SMILES: OB(O)c1c2ccccc2c(-c2ccccc2)c2ccccc12] | 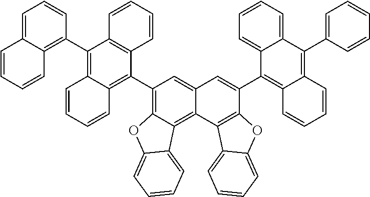 [SMILES: c1ccc(-c2c3ccccc3c(-c3cc4cc(-c5c6ccccc6c(-c6cccc7ccccc67)c6ccccc56)c5oc6ccccc6c5c4c4c3oc3ccccc34)c3ccccc23)cc1] |
| BB-13-f | BB-12-f | 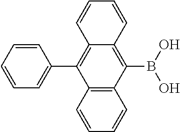 [SMILES: OB(O)c1c2ccccc2c(-c2ccccc2)c2ccccc12] | 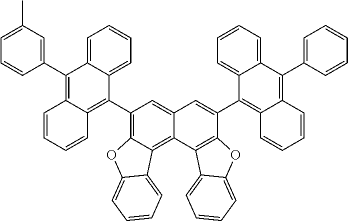 [SMILES: Cc1cccc(-c2c3ccccc3c(-c3cc4cc(-c5c6ccccc6c(-c6ccccc6)c6ccccc56)c5oc6ccccc6c5c4c4c3oc3ccccc34)c3ccccc23)c1] |
| BB-13-g | BB-12 | 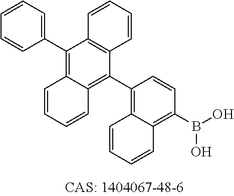 [SMILES: OB(O)c1ccc(-c2c3ccccc3c(-c3ccccc3)c3ccccc23)c2ccccc12] | 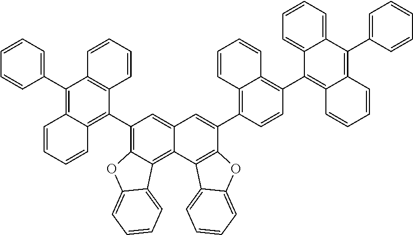 [SMILES: c1ccc(-c2c3ccccc3c(-c3ccc(-c4cc5cc(-c6c7ccccc7c(-c7ccccc7)c7ccccc67)c6oc7ccccc7c6c5c5c4oc4ccccc45)c4ccccc34)c3ccccc23)cc1] |
| BB-13-h | BB-12-b | 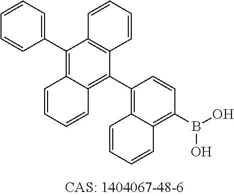 [SMILES: OB(O)c1ccc(-c2c3ccccc3c(-c3ccccc3)c3ccccc23)c2ccccc12] | 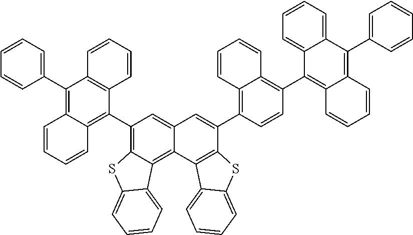 [SMILES: c1ccc(-c2c3ccccc3c(-c3ccc(-c4cc5cc(-c6c7ccccc7c(-c7ccccc7)c7ccccc67)c6sc7ccccc7c6c5c5c4sc4ccccc45)c4ccccc34)c3ccccc23)cc1] |

### Synthesis of BB-14

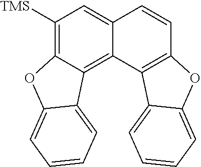 [SMILES: C[Si](C)(C)c1cc2ccc3oc4ccccc4c3c2c2c1oc1ccccc12]

Under an argon atmosphere, an oven dried flask is equipped with a magnetic stir bar, BB-6 (14.0 g, 43.1 mmol, 1.0 equiv.). THF (250 mL) is added and the reaction mixture is cooled to −78° C. n-BuLi (2.5 M in hexane, 22.4 mL, 56.1 mmol, 1.3 equiv.) is added. The reaction mixture is stirred for 1 h at −78° C. Trimethylsilyl chloride (24.8 mL, 194.1 mmol, 4.5 equiv.) is added. The reaction mixture is warmed overnight to rt. The raw product is purified by column chromatography. The desired product is obtained as white solid (16.4 g, 43.1 mmol, 99.9%).

Following compounds can be synthesized in analogous manner:

| | Starting | | |
|---|---|---|---|
| | material | reagent | Product |
| BB-14-b | BB-9 | TMS-Cl | 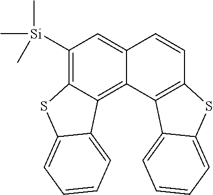 [SMILES: C[Si](C)(C)c1cc2ccc3sc4ccccc4c3c2c2c1sc1ccccc12] |
| BB-14-c | BB-6 | Trimethylborate | 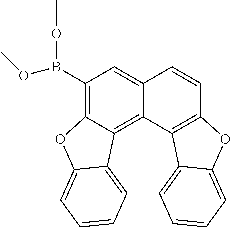 [SMILES: COB(OC)c1cc2ccc3oc4ccccc4c3c2c2c1oc1ccccc12] |

### Synthesis of BB-15

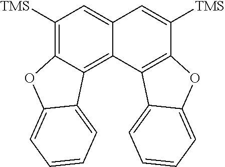 [SMILES: C[Si](C)(C)c1cc2cc([Si](C)(C)C)c3oc4ccccc4c3c2c2c1oc1ccccc12]

Under an argon atmosphere, an oven dried flask is equipped with a magnetic stir bar, BB-14 (16.3 g, 42.8 mmol, 1.0 equiv.). THF (200 mL) is added and the reaction mixture is cooled to −78° C. n-BuLi (2.5 M in hexane, 22.3 mL, 55.7 mmol, 1.3 equiv.) is added. The reaction mixture is stirred for 1 h at −78° C. Trimethylsilyl chloride (27.4 mL, 214.2 mmol, 5.0 equiv.) is added. The reaction mixture is warmed overnight to rt. The raw product is purified by column chromatography. The desired product is obtained as white solid (12.4 g, 27.4 mmol, 63.9%).

Following compounds can be synthesized in analogous manner:

| | Starting | | |
|---|---|---|---|
| | material | reagent | Product |
| BB-15-b | BB-14-b | TMS-Cl | 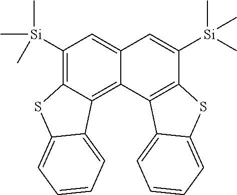 |

### Synthesis of BB-16

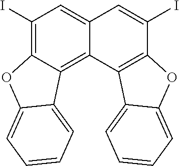 [SMILES: Ic1cc2cc(I)c3oc4ccccc4c3c2c2c1oc1ccccc12]

Under an argon atmosphere, an oven dried flask is equipped with a magnetic stir bar and BB-15 (11.8 g, 26.1 mmol, 1.0 equiv.). DCM (50 mL) is added and the resulting mixture is cooled down to 0° C. Iodmonochlorid (3.0 mL, 57.4 mmol, 2.2 equiv.) is added via syringe. Excess of Iodmonochlorid is quenched by the addition of saturated sodium thiosulfate solution (200 mL). The resulting mixture is dilute with toluene (300 mL). The organic phase is separated and concentrated under reduced pressure. The desired product is obtained as white solid (14.5 g, 25.9 mmol, 99.3%).

Following compounds can be synthesized in analogous manner:

| | Starting material | Product |
|---|---|---|
| BB-16-b | BB-15-b | 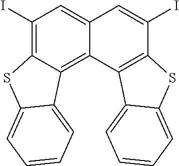 [SMILES: Ic1cc2cc(I)c3sc4ccccc4c3c2c2c1sc1ccccc12] |

### Synthesis of BB-17

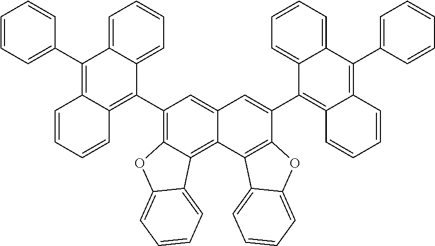 [SMILES: c1ccc(-c2c3ccccc3c(-c3cc4cc(-c5c6ccccc6c(-c6ccccc6)c6ccccc56)c5oc6ccccc6c5c4c4c3oc3ccccc34)c3ccccc23)cc1]

Under an argon atmosphere, an oven dried flask is equipped with a magnetic stir bar, 1,4-di-iodo-napthobisbenzofurane, (10.0 g, 17.9 mmol, 1.0 equiv.), (10-phenyl-9-anthryl) boronic acid (29.3 g, 5.5 mmol, 5.5 equiv.), (2-Dicyclohexylphosphino-2′,6′-dimethoxybiphenyl) [2-(2′-amino-1,1′-biphenyl)]palladium(II) methanesulfonate (2.8 g, 3.6 mmol, 0.2 equiv.) and potassium fluoride (6.2 g, 107.1 mmol, 6.0 equiv.). Toluene (300 mL), 1.4-dioxane (300 mL) and water (300 mL) is added and the mixture is refluxed overnight. The raw product is purified by column chromatography. The desired product is isolated as white solid (5.0 g, 6.2 mmol, 34.5%).

Following compounds can be synthesized in analogous manner:

| | Starting | | |
|---|---|---|---|
| | material | Starting material | Product |
| BB-17-b | BB-16-b | 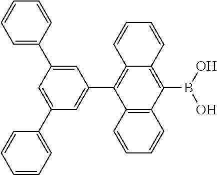 [SMILES: OB(O)c1c2ccccc2c(-c2cc(-c3ccccc3)cc(-c3ccccc3)c2)c2ccccc12] | 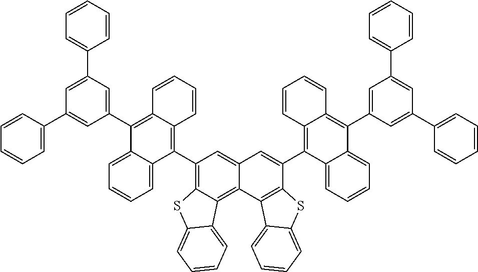 [SMILES: c1ccc(-c2cc(-c3ccccc3)cc(-c3c4ccccc4c(-c4cc5cc(-c6c7ccccc7c(-c7cc(-c8ccccc8)cc(-c8ccccc8)c7)c7ccccc67)c6sc7ccccc7c6c5c5c4sc4ccccc45)c4ccccc34)c2)cc1] |
| BB-17-c | BB-16-b | 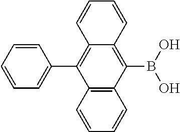 [SMILES: OB(O)c1c2ccccc2c(-c2ccccc2)c2ccccc12] | 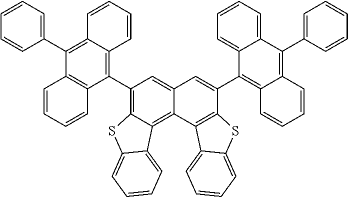 [SMILES: c1ccc(-c2c3ccccc3c(-c3cc4cc(-c5c6ccccc6c(-c6ccccc6)c6ccccc56)c5sc6ccccc6c5c4c4c3sc3ccccc34)c3ccccc23)cc1] |
| BB-17-d | BB-16 | 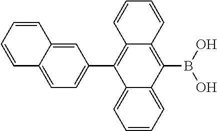 [SMILES: OB(O)c1c2ccccc2c(-c2ccc3ccccc3c2)c2ccccc12] | 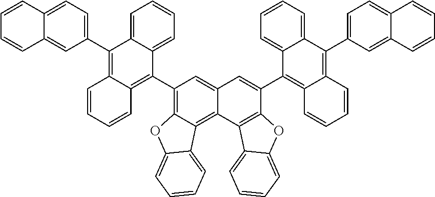 [SMILES: c1ccc2cc(-c3c4ccccc4c(-c4cc5cc(-c6c7ccccc7c(-c7ccc8ccccc8c7)c7ccccc67)c6oc7ccccc7c6c5c5c4oc4ccccc45)c4ccccc34)ccc2c1] |
| BB-17-e | BB-16 | 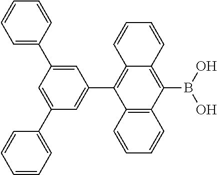 [SMILES: OB(O)c1c2ccccc2c(-c2cc(-c3ccccc3)cc(-c3ccccc3)c2)c2ccccc12] | 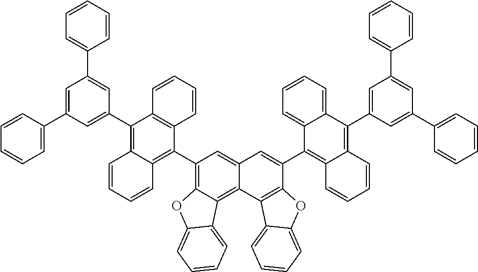 [SMILES: c1ccc(-c2cc(-c3ccccc3)cc(-c3c4ccccc4c(-c4cc5cc(-c6c7ccccc7c(-c7cc(-c8ccccc8)cc(-c8ccccc8)c7)c7ccccc67)c6oc7ccccc7c6c5c5c4oc4ccccc45)c4ccccc34)c2)cc1] |
| BB-17-f | BB-16 | 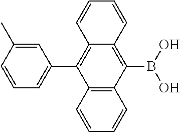 [SMILES: Cc1cccc(-c2c3ccccc3c(B(O)O)c3ccccc23)c1] | 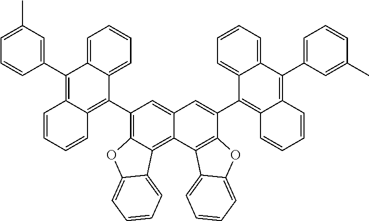 [SMILES: Cc1cccc(-c2c3ccccc3c(-c3cc4cc(-c5c6ccccc6c(-c6cccc(C)c6)c6ccccc56)c5oc6ccccc6c5c4c4c3oc3ccccc34)c3ccccc23)c1] |
| BB-17-g | BB-16 | 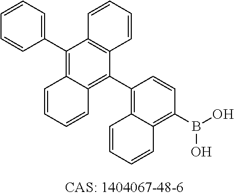 [SMILES: OB(O)c1ccc(-c2c3ccccc3c(-c3ccccc3)c3ccccc23)c2ccccc12] | 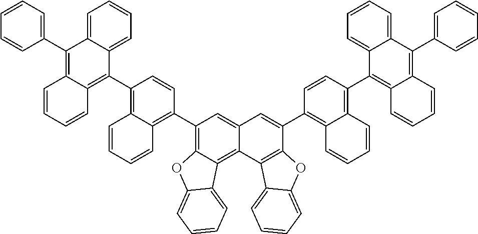 [SMILES: c1ccc(-c2c3ccccc3c(-c3ccc(-c4cc5cc(-c6ccc(-c7c8ccccc8c(-c8ccccc8)c8ccccc78)c7ccccc67)c6oc7ccccc7c6c5c5c4oc4ccccc45)c4ccccc34)c3ccccc23)cc1] |
| BB-17-h | BB-16 | 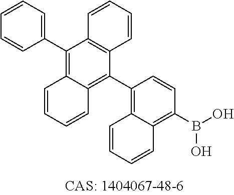 [SMILES: OB(O)c1ccc(-c2c3ccccc3c(-c3ccccc3)c3ccccc23)c2ccccc12] | 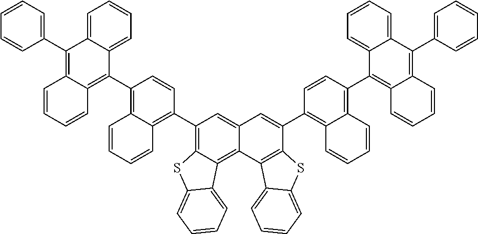 [SMILES: c1ccc(-c2c3ccccc3c(-c3ccc(-c4cc5cc(-c6ccc(-c7c8ccccc8c(-c8ccccc8)c8ccccc78)c7ccccc67)c6sc7ccccc7c6c5c5c4sc4ccccc45)c4ccccc34)c3ccccc23)cc1] |

### B) Fabrication of OLEDs

Fabrication of Vapor Processed OLED Devices

The manufacturing of the OLED devices is performed accordingly to WO 04/05891 with adapted film thicknesses and layer sequences. The following examples V1, E1, E2, E3, E4 and E5 show data of various OLED devices.

Substrate Pre-Treatment of Examples V1, E1 to E5:

Glass plates with structured ITO (50 nm, indium tin oxide) are coated with 20 nm PEDOT:PSS (Poly(3,4-ethylenedioxythiophene) poly(styrene-sulfonate, CLEVIOS™ P VP Al 4083 from Heraeus Precious Metals GmbH Germany, spin-coated from a water-based solution) to form the substrates on which the OLED devices are fabricated.

The OLED devices have in principle the following layer structure:
Substrate,
ITO (50 nm),
Buffer (20 nm),
Hole transporting layer (HTL),
Interlayer (IL),
Electron blocking layer (EBL),
Emissive layer (EML),
Electron transporting layer (ETL),
Cathode.

The cathode is formed by an aluminium layer with a thickness of 100 nm. The detailed stack sequence is shown in table A. The materials used for the OLED fabrication are presented in table C.

All materials are applied by thermal vapour deposition in a vacuum chamber. The emission layer here always consists of at least one matrix material (host material=H) and an emitting dopant (emitter=D), which is mixed with the matrix material or matrix materials in a certain proportion by volume by co-evaporation. An expression such as H1:D1 (95%:5%) here means that material H1 is present in the layer in a proportion by volume of 95%, whereas D1 is present in the layer in a proportion of 5%. Analogously, the electron-transport layer may also consist of a mixture of two or more materials.

The OLED devices are characterised by standard methods. For this purpose, the electroluminescence spectra, the current efficiency (measured in cd/A), power efficiency (Im/W) and the external quantum efficiency (EQE, measured in % at 1000 cd/m²) are determined from current/voltage/luminance characteristic lines (IUL characteristic lines) assuming a Lambertian emission profile. The electroluminescence (EL) spectra are recorded at a luminous density of 1000 cd/m² and the CIE 1931 x and y coordinates are then calculated from the EL spectrum. U1000 is defined as the voltage at luminous density of 1000 cd/m². SE1000 represents the current efficiency, LE1000 the power efficiency at 1000 cd/m². EQE1000 is defined as the external quantum efficiency at luminous density of 1000 cd/m².

The device data of various OLED devices are summarized in table B. The example V1 represents the comparative example according to the state-of-the-art. The examples E1 to E5 show data of inventive OLED devices.

In the following section several examples are described in more detail to show the advantages of the inventive OLED devices.

Use of Inventive Compounds as Host Material in Fluorescent OLEDs

The inventive compounds are especially suitable as a host (matrix) when blended with a fluorescent blue dopant (emitter) to form the emissive layer of a fluorescent blue OLED device. The representative examples are H1, H2, H3, H4 and H5. Comparative compound for the state-of-the-art is represented by SdT (structures see table C). The use of the inventive compound as a host (matrix) in a fluorescent blue OLED device results in excellent device data, especially with respect to power efficiency (LE1000) when compared to the state-of-the-art (compare E1 to E5 versus V1, see device data see table B).

TABLE A
device stack of vapor processed OLEDs

| | HTL | IL | EBL | EML | ETL |
|---|---|---|---|---|---|
| Ex. | [nm] | [nm] | [nm] | [nm] | [nm] |
| V1 | SpA | HATCN | SpMA | SdT:D1 | ETM:LiQ |
| | 140 nm | 5 nm | 20 nm | (95%:5%) 20 nm | (50%:50%) |
| | | | | | 30 nm |
| El | SpA | HATCN | SpMA | Hl:D1 | ETM:LiQ |
| | 140 nm | 5 nm | 20 nm | (95%:5%) 20 nm | (50%:50%) |
| | | | | | 30 nm |
| E2 | SpA | HATCN | SpMA | H2:D1 | ETM:LiQ |
| | 140 nm | 5 nm | 20 nm | (95%:5%) 20 nm | (50%:50%) |
| | | | | | 30 nm |
| E3 | SpA | HATCN | SpMA | H3:D1 | ETM:LiQ |
| | 140 nm | 5 nm | 20 nm | (95%:5%) 20 nm | (50%:50%) |
| | | | | | 30 nm |
| E4 | SpA | HATCN | SpMA | H4:D1 | ETM:LiQ |
| | 140 nm | 5 nm | 20 nm | (95%:5%) 20 nm | (50%:50%) |
| | | | | | 30 nm |
| E5 | SpA | HATCN | SpMA | H5:D1 | ETM:LiQ |
| | 140 nm | 20 nm | 5 nm | (95%:5%) 20 nm | (50%:50%) |
| | | | | | 30 nm |

TABLE B
device data of vapor processed OLEDs

| | U1000 | SE1000 | LE1000 | EQE1000 | ClE x/y at |
|---|---|---|---|---|---|
| Bsp. | (V) | (cd/A) | (Im/W) | ([%]) | 1000 cd/m² |
| V | 5.5 | 7.2 | 4.1 | 6.0 | 0.13/0.14 |
| E1 | 4.9 | 7.8 | 5.0 | 6.7 | 0.13/0.14 |
| E2 | 5.0 | 8.1 | 5.1 | 6.9 | 0.13/0.13 |
| E3 | 4.8 | 7.5 | 4.9 | 6.6 | 0.13/0.14 |
| E4 | 5.1 | 82 | 5.1 | 7.0 | 0.13/0.13 |
| E5 | 4.9 | 7.9 | 5.1 | 6.7 | 0.13/0.14 |

TABLE C
Structural formulae of vapor processed OLED materials

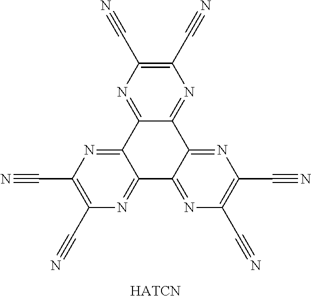

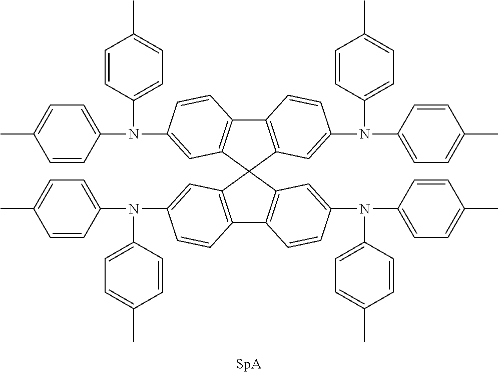

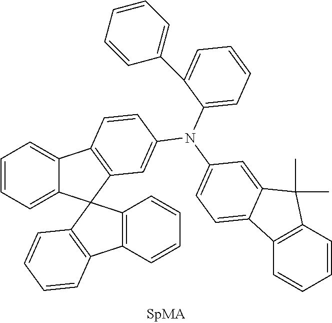 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

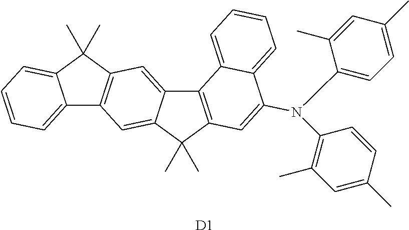 [SMILES: Cc1ccc(N(c2ccc(C)cc2C)c2cc3c(c4ccccc24)-c2cc4c(cc2C3(C)C)-c2ccccc2C4(C)C)c(C)c1 ; "D1"]

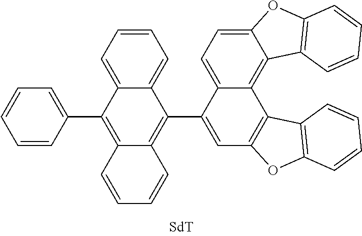 [SMILES: c1ccc(-c2c3ccccc3c(-c3cc4oc5ccccc5c4c4c3ccc3oc5ccccc5c34)c3ccccc23)cc1]

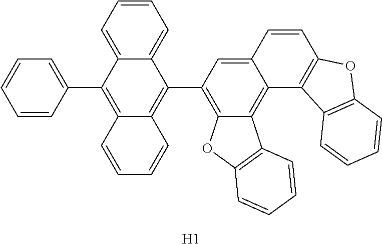 [SMILES: c1ccc(-c2c3ccccc3c(-c3cc4ccc5oc6ccccc6c5c4c4c3oc3ccccc34)c3ccccc23)cc1 ; "H1"]

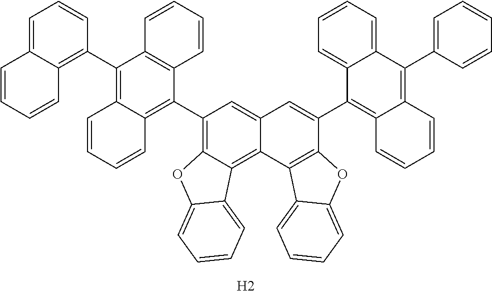

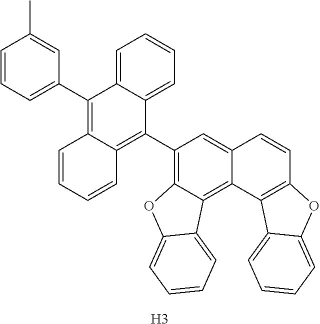

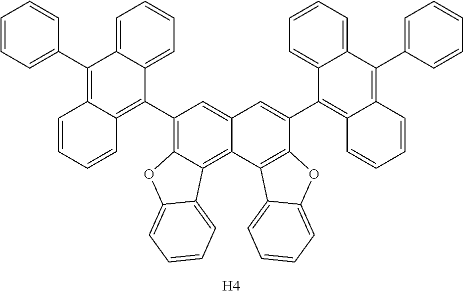

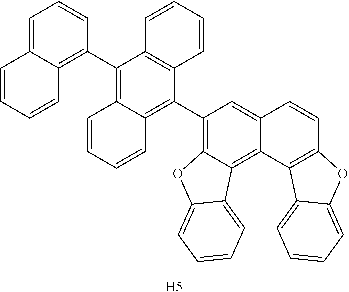

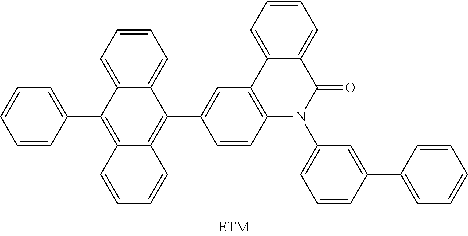 [SMILES: O=c1c2ccccc2c2cc(-c3c4ccccc4c(-c4ccccc4)c4ccccc34)ccc2n1-c1cccc(-c2ccccc2)c1]

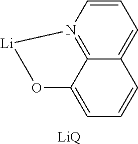

1.-15. (canceled)

16. A compound of the formula (1), 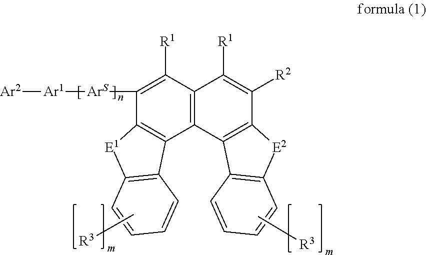
where the following applies to the symbols and indices used:
Ar¹ is on each occurrence, identically or differently, a condensed aryl or heteroaryl group having 10 to 18 aromatic ring atoms, which may be substituted by one or more radicals R;
Ar² is on each occurrence, identically or differently, an aromatic or heteroaromatic ring system having 5 to 60 aromatic ring atoms, which may in each case be substituted by one or more radicals R;
Ar^{S} is on each occurrence, identically or differently, an aromatic or heteroaromatic ring system having 5 to 30 aromatic ring atoms, which may in each case be substituted by one or more radicals R;
E¹, E² are on each occurrence, identically or differently, selected from —BR⁰—, —C(R⁰)₂—, —Si(R⁰)₂—, —C(═O)—, —O—, —S—, —S(═O)—, —SO₂—, —N(R⁰)—, and —P(R⁰)—;
R¹ stands on each occurrence, identically or differently, for H, D, F, Cl, Br, I, CHO, CN, N(Ar)₂, C(═O)Ar, P(═O)(Ar)₂, S(═O)Ar, S(═O)₂Ar, NO₂, Si(R)₃, B(OR)₂, OSO₂R, a straight-chain alkyl, alkoxy or thioalkyl group having 1 to 40 C atoms or branched or cyclic alkyl, alkoxy or thioalkyl group having 3 to 40 C atoms, each of which may be substituted by one or more radicals R, where in each case one or more non-adjacent CH₂ groups may be replaced by RC═CR, C≡C, Si(R)₂, Ge(R)₂, Sn(R)₂, C═O, C═S, C═Se, P(═O)(R), SO, SO₂, O, S or CONR and where one or more H atoms may be replaced by D, F, Cl, Br, I, CN or NO₂, an aromatic or heteroaromatic ring system having 5 to 60 aromatic ring atoms, which may in each case be substituted by one or more radicals R, or an aryloxy group having 5 to 60 aromatic ring atoms, which may be substituted by one or more radicals R, where two substituents R¹ may form a mono- or polycyclic, aliphatic ring system or aromatic ring system, which may be substituted by one or more radicals R;
R², R³ stand on each occurrence, identically or differently, for
H, D, F, Cl, Br, I, CHO, CN, N(Ar)₂, C(═O)Ar, P(═O)(Ar)₂, S(═O)Ar, S(═O)₂Ar, NO₂, Si(R)₃, B(OR)₂, OSO₂R;
a straight-chain alkyl, alkoxy or thioalkyl group having 1 to 40 C atoms or branched or cyclic alkyl, alkoxy or thioalkyl group having 3 to 40 C atoms, each of which may be substituted by one or more radicals R, where in each case one or more non-adjacent CH₂ groups may be replaced by RC═CR, C≡C, Si(R)₂, Ge(R)₂, Sn(R)₂, C═O, C═S, C═Se, P(═O)(R), SO, SO₂, O, S or CONR and where one or more H atoms may be replaced by D, F, Cl, Br, I, CN or NO₂;
an aromatic or heteroaromatic ring system having 5 to 60 aromatic ring atoms, which may in each case be substituted by one or more radicals R;
an aryloxy group having 5 to 60 aromatic ring atoms, which may be substituted by one or more radicals R, where one substituent R²; or
for a group of the following formula: 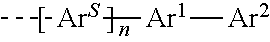
where the dashed bond indicates the bond to the structure of formula (1);
and where one adjacent substituent R¹ and/or two substituents R³ may form a mono- or polycyclic, aliphatic ring system or aromatic ring system, which may be substituted by one or more radicals R;
m stands on each occurrence, identically or differently, for an integer selected from 0, 1, 2, 3 or 4;
n stands on each occurrence, identically or differently, for an integer selected from 0, 1, 2, 3 or 4;
R stands on each occurrence, identically or differently, for H, D, F, Cl, Br, I, CHO, CN, N(Ar)₂, C(═O)Ar, P(═O)(Ar)₂, S(═O)Ar, S(═O)₂Ar, NO₂, Si(R′)₃, B(OR′)₂, OSO₂R, a straight-chain alkyl, alkoxy or thioalkyl group having 1 to 40 C atoms or branched or cyclic alkyl, alkoxy or thioalkyl group having 3 to 40 C atoms, each of which may be substituted by one or more radicals R′, where in each case one or more non-adjacent CH₂ groups may be replaced by R′C═CR′, C≡C, Si(R′)₂, Ge(R′)₂, Sn(R′)₂, C═O, C═S, C═Se, P(═O)(R′), SO, SO₂, O, S or CONR′ and where one or more H atoms may be replaced by D, F, Cl, Br, I, CN or NO₂, an aromatic or heteroaromatic ring system having 5 to 60 aromatic ring atoms, which may in each case be substituted by one or more radicals R′, or an aryloxy group having 5 to 60 aromatic ring atoms, which may be substituted by one or more radicals R′, where two adjacent substituents R may form a mono- or polycyclic, aliphatic ring system or aromatic ring system, which may be substituted by one or more radicals R′;
Ar is an aromatic or heteroaromatic ring system having 5 to 24 aromatic ring atoms, which may in each case also be substituted by one or more radicals R′;
R stands on each occurrence, identically or differently, for H, D, F, Cl, Br, I, CN, a straight-chain alkyl, alkoxy or thioalkyl group having 1 to 20 C atoms or branched or cyclic alkyl, alkoxy or thioalkyl group having 3 to 20 C atoms, where in each case one or more non-adjacent CH₂ groups may be replaced by SO, SO₂, O, S and where one or more H atoms may be replaced by D, F, Cl, Br or I, or an aromatic or heteroaromatic ring system having 5 to 24 C atoms.

17. The compound according to claim 16, wherein the compound is selected from the compounds of formula (2) or (3), 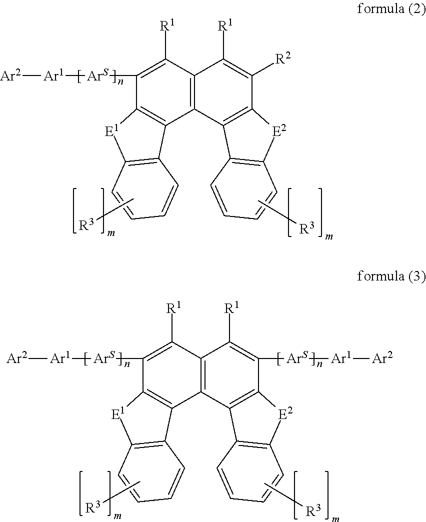
where
R², R³ stand on each occurrence, identically or differently, for H, D, F, Cl, Br, I, CHO, CN, N(Ar)₂, C(═O)Ar, P(═O)(Ar)₂, S(═O)Ar, S(═O)₂Ar, NO₂, Si(R)₃, B(OR)₂, OSO₂R, a straight-chain alkyl, alkoxy or thioalkyl group having 1 to 40 C atoms or branched or cyclic alkyl, alkoxy or thioalkyl group having 3 to 40 C atoms, each of which may be substituted by one or more radicals R, where in each case one or more non-adjacent CH₂ groups may be replaced by RC═CR, C═C, Si(R)₂, Ge(R)₂, Sn(R)₂, C═O, C═S, C═Se, P(═O)(R), SO, SO₂, O, S or CONR and where one or more H atoms may be replaced by D, F, Cl, Br, I, CN or NO₂, an aromatic or heteroaromatic ring system having 5 to 60 aromatic ring atoms, which may in each case be substituted by one or more radicals R, or an aryloxy group having 5 to 60 aromatic ring atoms, which may be substituted by one or more radicals R, where one substituent R² and one adjacent substituent R¹ and/or two substituents R³ may form a mono- or polycyclic, aliphatic ring system or aromatic ring system, which may be substituted by one or more radicals R; and
where the symbols R¹, E¹, E², Ar¹, Ar² and Ar^{S} and the indices m and n have the same meaning as in claim 16.

18. The compound according to claim 16, wherein the group Ar¹ is on each occurrence, identically or differently, selected from the group consisting of anthracene, naphthalene, phenanthrene, tetracene, chrysene, benzanthracene, benzo-phenanthracene, pyrene, perylene, triphenylene, benzopyrene, fluoranthene, each of which may be substituted by one or more radicals R at any free positions.

19. The compound according to claim 16, wherein the group Ar is selected from the groups of formulae (Ar1-1) to (Ar1-11), 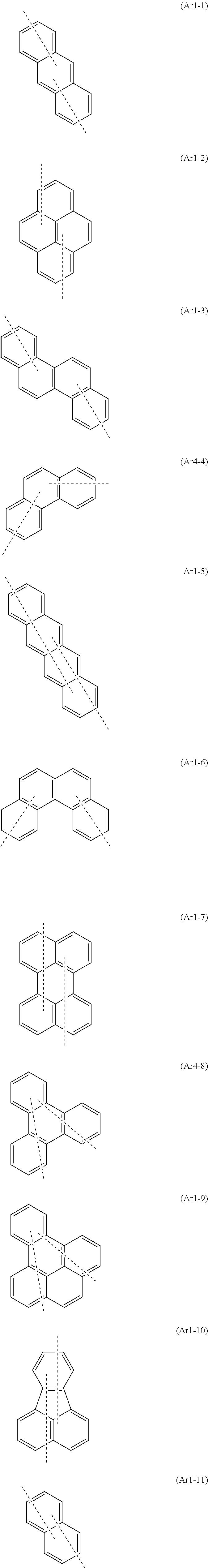
where
the dashed bonds indicate the bonding to the adjacent group in formula (1); and where the groups of formulae (Ar1-1) to (Ar1-11) may be substituted at each free position by a group R, which has the same meaning as in claim 16.

20. The compound according to claim 16, wherein the compound is selected from the compounds of formula (2-1) or (3-1), 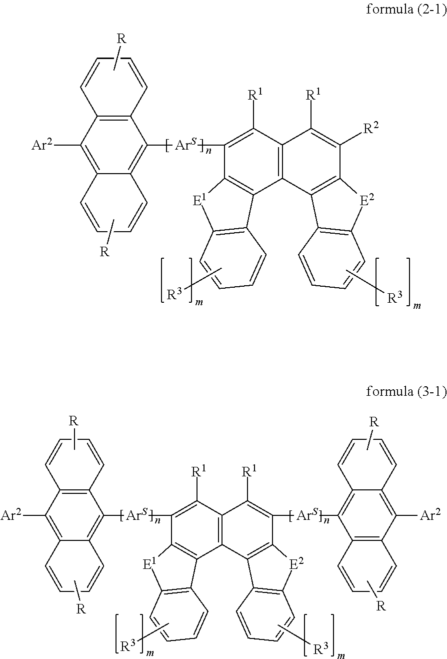
where
R², R³ stand on each occurrence, identically or differently, for H, D, F, Cl, Br, I, CHO, CN, N(Ar)₂, C(═O)Ar, P(═O)(Ar)₂, S(═O)Ar, S(═O)₂Ar, NO₂, Si(R)₃, B(OR)₂, OSO₂R, a straight-chain alkyl, alkoxy or thioalkyl group having 1 to 40 C atoms or branched or cyclic alkyl, alkoxy or thioalkyl group having 3 to 40 C atoms, each of which may be substituted by one or more radicals R, where in each case one or more non-adjacent CH₂ groups may be replaced by RC═CR, C≡C, Si(R)₂, Ge(R)₂, Sn(R)₂, C═O, C═S, C═Se, P(═O)(R), SO, SO₂, O, S or CONR and where one or more H atoms may be replaced by D, F, Cl, Br, I, CN or NO₂, an aromatic or heteroaromatic ring system having 5 to 60 aromatic ring atoms, which may in each case be substituted by one or more radicals R, or an aryloxy group having 5 to 60 aromatic ring atoms, which may be substituted by one or more radicals R, where one substituent R² and one adjacent substituent R¹ and/or two substituents R³ may form a mono- or polycyclic, aliphatic ring system or aromatic ring system, which may be substituted by one or more radicals R; and
where the symbols R, R¹, E¹, E², Ar² and Ar^{S} and the indices m and n have the same meaning as above.

21. The compound according to claim 16, wherein the group Ar^{S} stands on each occurrence, identically or differently, for phenyl, biphenyl, fluorene, spirobifluorene, naphthalene, phenanthrene, anthracene, dibenzofuran, dibenzothiophene, carbazole, pyridine, pyrimidine, pyrazine, pyridazine, triazine, benzopyridine, benzopyridazine, benzopyrimidine and quinazoline, each of which may be substituted by one or more radicals R.

22. The compound according to claim 16, wherein the compound is selected from the compounds of formulae (2-1-1) to (3-1-6), 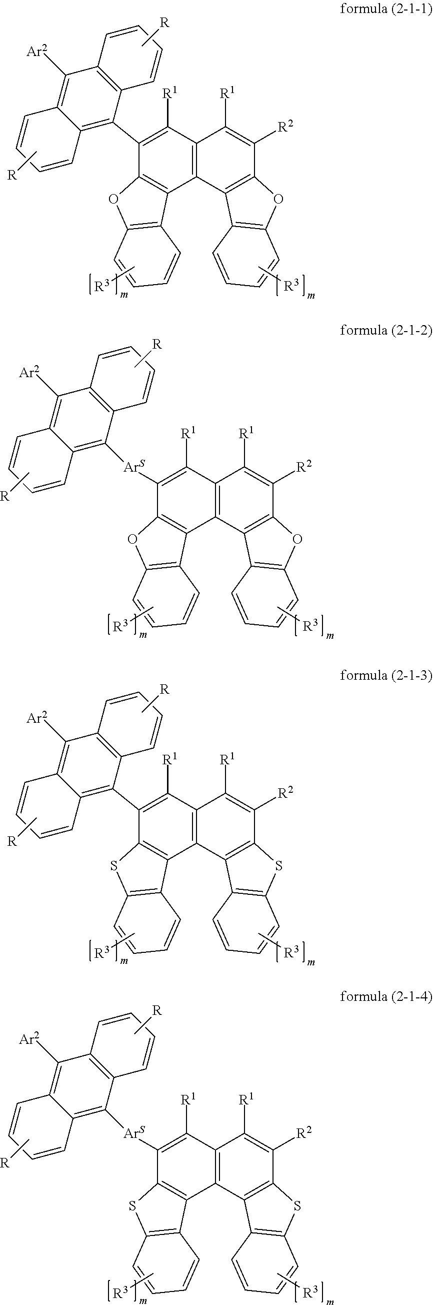 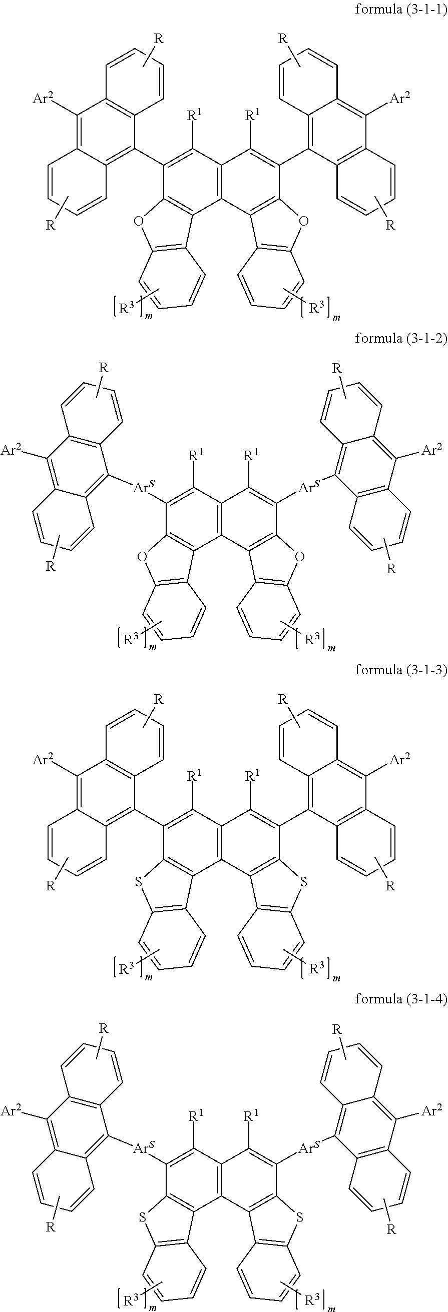 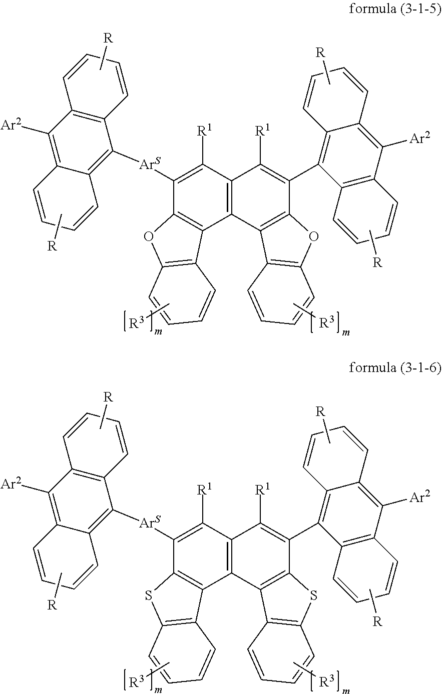
where
the symbols R², R³ have the same meaning as in claim 20, the symbols R, R¹, Ar² and Ar^{S} and the index m have the same meaning as in claim 16.

23. The compound according to claim 16, wherein Ar² is selected from the group consisting of phenyl, biphenyl, terphenyl, quaterphenyl, fluorene, spirobifluorene, naphthalene, phenanthrene, anthracene, triphenylene, fluoranthene, tetracene, chrysene, benzanthracene, benzophenanthracene, pyrene, perylene, indole, benzofuran, benzothiophene, dibenzofuran, dibenzothiophene, carbazole, indenocarbazole, indolocarbazole, pyridine, pyrimidine, pyrazine, pyridazine, triazine, quinolone, benzopyridine, benzopyridazine, benzopyrimidine, benzimidazole and quinazoline, each of which may be substituted by one or more radicals R.

24. The compound according to claim 16, wherein the compound is selected from the compounds of formulae (2-1-5) to (3-1-12), 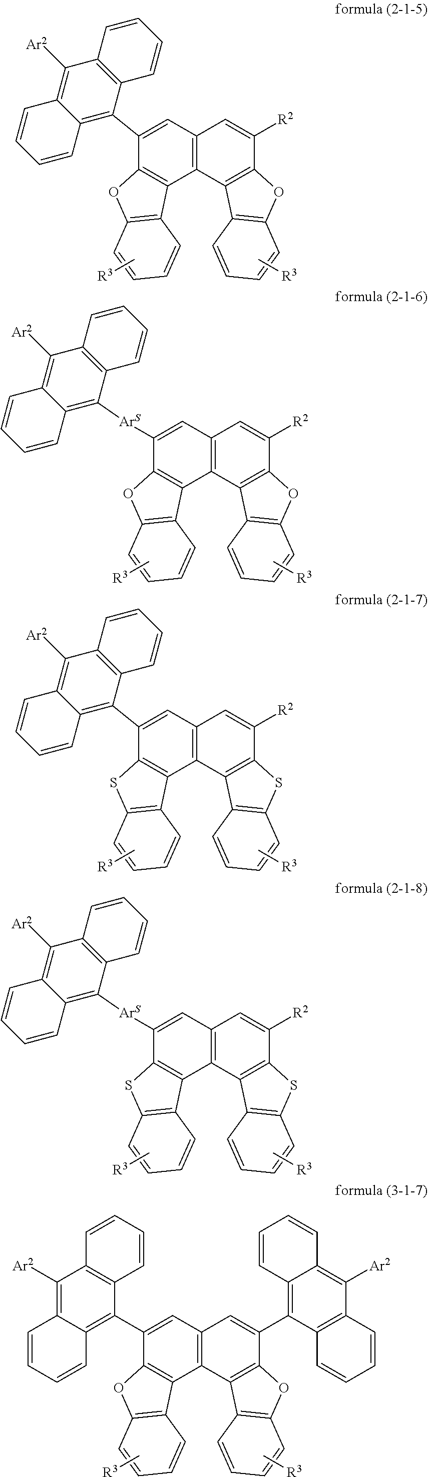 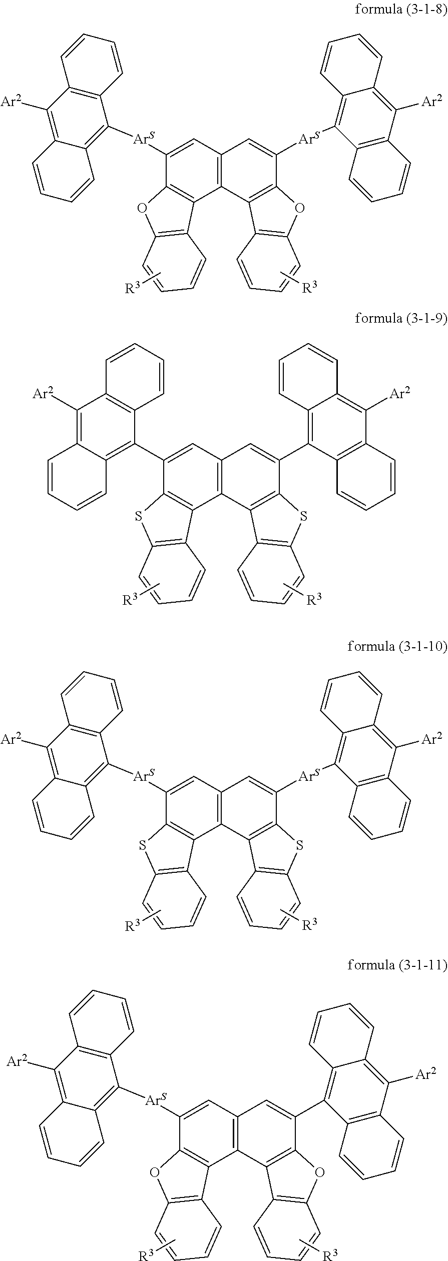 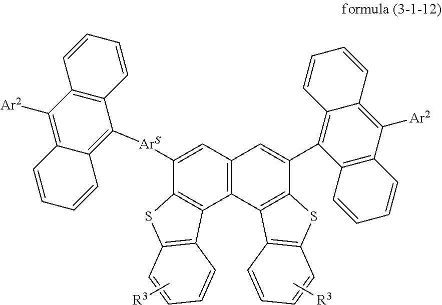
where
the symbols R², R³ have the same meaning as in claim 20, and the symbols R, R¹, Ar² and Ar^{S} have the same meaning as in claim 16.

25. A process for the preparation of the compound according to formula (1) as defined in claim 16, where the process comprises one of the following synthesis routes a1), a2), a3) or a4):
Route a1): 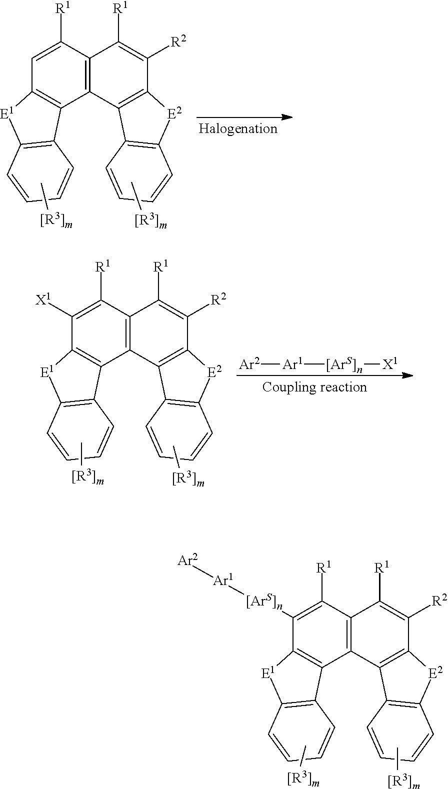
Route a2): 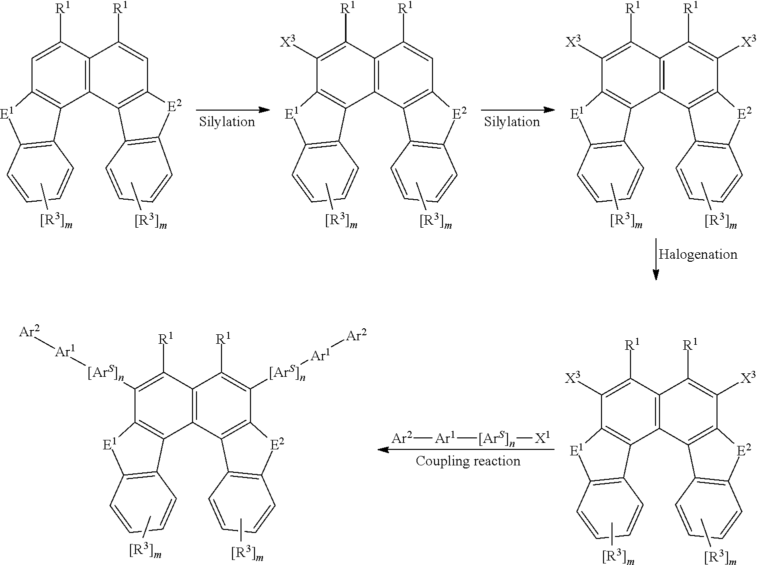
Route a3): 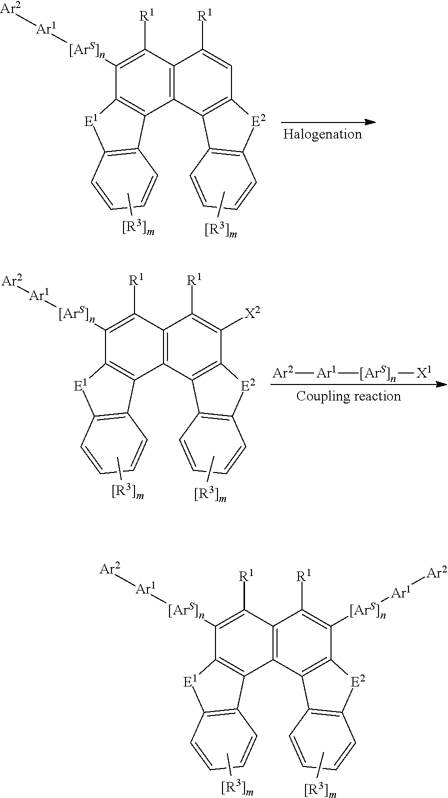
Route a4): 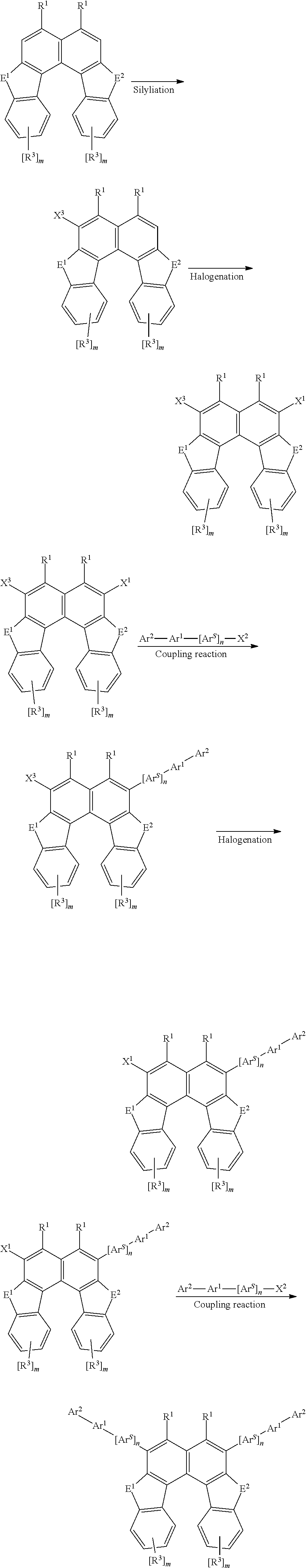
where the symbols R¹, R², R³, Ar¹, Ar², Ar^{S} have the same meaning as above, and where:
X¹ is a leaving group selected from halogens and triflate;
X² is a leaving group selected from boronic acids and boronic esters;
X³ is a leaving group selected from silyl groups.

26. A compound of formulae (Int-1), (Int-2), (Int-3), (Int-4) and (Int-5), 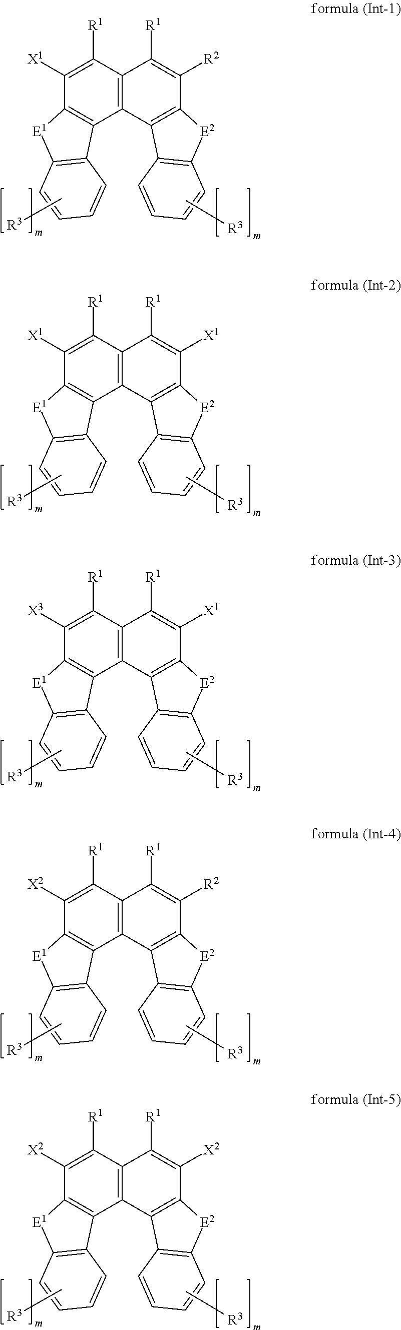
where the symbols R¹, R², R³, E¹ and E² have the same meaning as in claim 16 and the symbols X¹ and X³ have the same meaning as in claim 25.

27. A formulation comprising at least one compound according to claim 16 and at least one solvent.

28. A polymer, oligomer or dendrimer containing one or more compounds according to claim 16, where the bond(s) to the polymer, oligomer or dendrimer may be localised at any positions in formula (1) which is substituted by R, R¹, R² or R³.

29. An electronic device comprising at least one compound according to claim 16 selected from the group consisting of organic electroluminescent devices, organic integrated circuits, organic field-effect transistors, organic thin-film transistors, organic light-emitting transistors, organic solar cells, dye-sensitised organic solar cells, organic optical detectors, organic photoreceptors, organic field-quench devices, light-emitting electrochemical cells, organic laser diodes and organic plasmon emitting devices.

30. An electronic device comprising at least one polymer, oligomer or dendrimer according to claim 28, selected from the group consisting of organic electroluminescent devices, organic integrated circuits, organic field-effect transistors, organic thin-film transistors, organic light-emitting transistors, organic solar cells, dye-sensitised organic solar cells, organic optical detectors, organic photoreceptors, organic field-quench devices, light-emitting electrochemical cells, organic laser diodes and organic plasmon emitting devices.

31. An organic electroluminescent device comprising the compound according to claim 16 is employed as a fluorescent emitter or as a matrix material for fluorescent emitters.

32. An organic electroluminescent device comprising the polymer, oligomer or dendrimer according to claim 28 is employed as a fluorescent emitter or as a matrix material for fluorescent emitters.
